(19) United States
(12) Patent Application Publication
(10) Pub. No.: US 2021/0205260 A1
(43) Pub. Date: Jul. 8, 2021

(54) MP53 RESCUE COMPONDS AND METHODS OF TREATING A P53 DISORDER

(71) Applicant: RUI JIN HOSPITAL, SHANGHAI JIAO TONG UNIVERSITY SCHOOL OF MEDICINE, Shanghai (CN)

(72) Inventors: Min LU, Shanghai (CN); Jiale WU, Shanghai (CN); Huaxin SONG, Shanghai (CN)

(21) Appl. No.: 16/959,894

(22) PCT Filed: Jan. 2, 2019
(86) PCT No.: PCT/CN2019/070117

§ 371 Date: Jul. 2, 2020

Related U.S. Application Data
(63) Continuation of application No. PCT/CN2018/085190, filed on Apr. 28, 2018.
Application No. PCT/CN2018/085190 is a continuation of application No. PCT/CN2018/070051, filed on Jan. 2, 2018.

(51) Int. Cl. A61K 31/285; A61K 31/29; A61K 33/36; A61K 33/245; A61K 33/243; A61K 45/06

(52) U.S. Cl. CPC A61K 31/285; A61K 31/29; A61K 45/06; A61K 33/245; A61K 33/243; A61K 33/36

(57) ABSTRACT

Novel mp53 rescue compounds and the pharmaceutical composition, and methods of treating a p53 disorder.

### TECHNICAL FIELD

Various compositions for the rescue of a mp53, various pharmaceutical composition for a p53 disorder, such as cancer, and various methods for treating the p53 disorder, are disclosed herein.

### CROSS REFERENCE TO RELATED APPLICATIONS

This application claims priority to International Application No. PCT/CN2018070051 filed on Jan. 2, 2018, entitled “PANDA AS A NOVEL THERAPEUTIC” and International Application No. PCT/CN/2018/085190 filed on Apr. 28, 2018, entitled “PANDA AS A NOVEL THERAPEUTIC,” the content of each application is incorporated herein by reference in their entirety.

### BACKGROUND

Various compounds for rescuing mp53 and treating a p53 disorder, including cancer, and various methods of treating a p53 disorder have been proposed. Because these compounds, treatments and methods of treatments are not optimal, there is a need in the field for improved mp53 rescue compounds, treatments for a p53 disorder, and methods of treating a p53 disorder.

### SUMMARY

We have described herein compounds that have one or more useful characteristic(s) and can form one or more tight association(s) with a PANDA Pocket (each compound a “PANDA Agent”). In certain embodiments, the PANDA Agent can regulate the level of one or more p53 target gene. Exemplary target genes include Apaf1, Bax, Fas, Dr5, mir-34, Noxa, TP53AIP1, Perp, Pidd, Pig3, Puma, Siva, YWHAZ, Btg2, Cdkn1a, Mdm2, Tp53i3, Gadd45a, mir-34a, mir-34b/34c, Prt3, Ptprv, Reprimo, Pai1, Pml, Ddb2, Ercc5, Fancc, Gadd45a, Ku86, Mgmt, Mih1, Msh2, P53r2, Polk, Xpc, Adora2b, Aldh4, Gamt Gs2, Gpx1, Lpin1, Parkin, Prkab1, Prkab2, Pten, Sco1, Sesn1, Sesn2, Tigar, Tp53inp1, Tsc2, Atg10, Atg2b, Atg4a, Atg4c, Atg7, Ctsd, Ddit4, Dram1, Foxo3, Laptm4a, Lkb1, Pik3r3, Prkag2, Puma, Tpp1, Tsc2, Ulk1, Ulk2, Uvrag, Vamp4, Vmp1, Bai1, Cx3cl1, Icam1, lrf5, lrf9, Isg15, Maspin, Mcp1, Ncf2, Pai1, Tlr1-Tlr10, Tsp1, Ulbp1, Ulbp2, mir-34a, mir-200c, mir-145, mir-34a, mir-34b/34c, Notch1, combinations thereof and the like. In certain embodiments, the tight association formed by PANDA Agent and PANDA Pocket substantially stabilizes p53. Preferably, the tight association increases the Tₘ of p53 at least by about 0.5° C., more preferably at least by about 1° C., further preferably at least by about 2° C., further preferably at least by about 5° C., further preferably at least to about 8° C. In certain embodiments, the tight association formed by PANDA Agent and PANDA Pocket increases the population of properly folded p53 at least to about 1.5 times, preferably at least to about 3 times, more preferably at least to about 5 times, more preferably at least to about 10 times, and further preferably to about 100 times. In preferred embodiments, the increase is measured to a PAb1620 immunoprecipitation assay.

In certain embodiments, the PANDA Agent includes one or more PANDA Pocket-binding groups capable of binding one or more amino acids on PANDA Pocket, preferably one or more cysteines, more preferably two or more cysteines, further preferably more than three cysteines, further preferably from about three cysteines to about 6 cysteines. The PANDA Pocket binding group is preferred to include metallic group(s), metalloid group(s), and other group(s) capable of binding to PANDA Pocket such as Michael acceptor(s) and thiol group(s). The PANDA Pocket-binding groups is further preferred to include one or more arsenic, antimony, and bismuth, including any analogue(s) thereof, and any combinations thereof. Exemplary PANDA Pocket-binding groups include compounds containing a 3-valence and/or 5-valence arsenic atom, a 3-valence and/or 5-valence antimony atom, a 3-valence and/or 5-valence bismuth atom, and/or a combination thereof.

Exemplary embodiments of a PANDA Agent can include any one of the following Formulas I-XV.

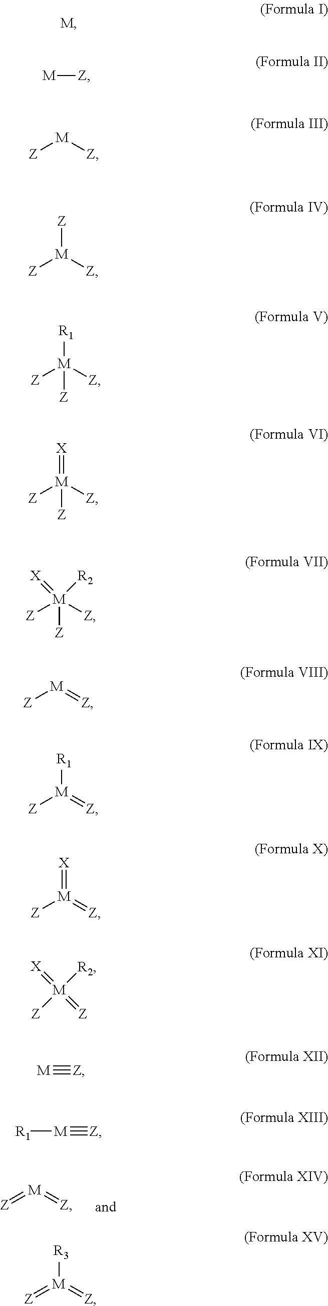

wherein:
M is an atom selected from a group consisting of As, Sb, and Bi;
Z is a functional group comprising a non-Carbon atom that forms a bond with M,
   wherein the non-Carbon atom is preferably selected from the group consisting of H, D, F, Cl, Br, I, O, S, Se, Te, Li, Na, K, Cs, Mg, Cu, Zn, Ba, Ta, W, Ag, Cd, Sn, X, B, N, P, Al, Ga, In, TI, Ni, Si, Ge, Cr, Mn, Fe, Co, Pb, Y, La, Zr, Nb, Pr, Nd, Sm, Eu, Gd, Dy, Tb, Ho, Er, Tm, Yb, and Lu;

wherein:
R₁ is selected from 1 to 9 X groups;
R₂ is selected from 1 to 7 X groups;
R₃ is selected from 1 to 8 X groups; and
wherein each X group comprises an atom that forms a bond with M; and

wherein:
each of M, the non-Carbon atom, and the atom has the appropriate charge, including no charge, in the compound;
each of Z and X is independently selected and can be the same or different from the other Z or X in the compound, respectively; and each of the M, non-Carbon atom and the atom can be a part of a ring member.

In the preferred embodiment, the non-Carbon atom is selected from the group consisting of O, S, N, X, F, C, Br, I, and H.

The following Equation (1) is an reaction for PANDA Agent. A compound containing M group with a Z₁ (a first group with the capacity to bind a first cysteine) and/or a Z₂ (a second group with the capacity to bind a second cysteine) and/or a Z₃ (a third group with the capacity to bind a third cysteine), Examples of Z₁, Z₂, and Z₃ includes O, S, N, X, F, Cl, Br, I, OH, and H. Z₁, Z₂, and/or Z₃ can bind to each other. M group includes for example a metal, such as an bismuth, a metalloid, such as an arsenic and an antimony, a group such as a Michael acceptor and/or a thiol, and/or any analogue with cysteine-binding ability. The PANDA Agent can undergo a hydrolysis before reacting and binding to p53 forming PANDA. In some cases, when a group cannot undergo hydrolysis, and accordingly cannot bind to a cysteine. In such cases, the remaining group(s) with cysteine binding potential binds to p53. X₁ and X₂ represent any groups bound to M. X₁ and/or X₂ can also be empty. X₁ and/or X₂ can also be able to bind cysteine.

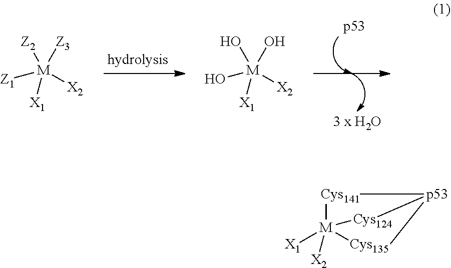

The following Equations (2) and (3) is an exemplary reaction for a PANDA Agent with tri-cysteine binding potential. 3-valence ATO or KAsO₂ undergoes hydrolysis, covalently binds to three PANDA Cysteines on p53.

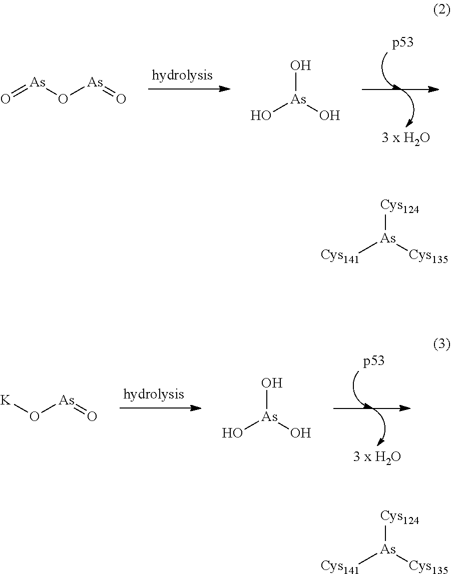

The following equation (4) is an exemplary reaction for a PANDA Agent with tri-cysteine binding potential. 5-valence As compound undergoes hydrolysis, covalently binds to three PANDA Cysteines on p53.

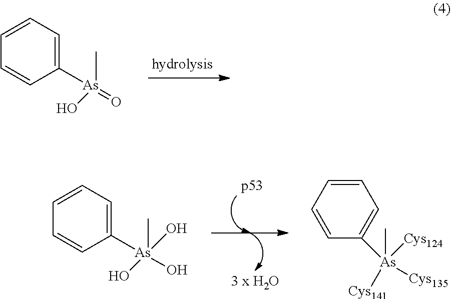

The following equation (5) is an exemplary reaction for a PANDA Agent with bi-cysteine binding potential. The PANDA Agent can bind to PANDA Cysteines, or to PANDA Cysteines (Cys₁₂₄, Cys₁₃₅, or Cys₁₄₁), or Cys₂₇₅ and Cys₂₇₇ or C₂₃₈ and C₂₄₂.

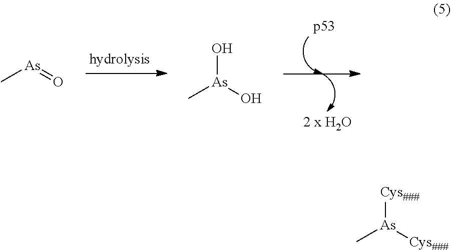

The following equation (6) is an exemplary reaction for a PANDA Agent with mono-cysteine binding potential. The PANDA Agent can bind to PANDA Cysteines, (i.e. Cys₁₂₄, Cys₁₃₅, or Cys₁₄₁) or the other 3 cysteines on PANDA Pocket (Cys₂38, Cys₂₇₅, or Cys₂₇7).

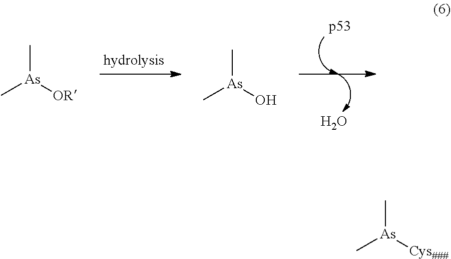

Exemplary PANDA Agent includes one or more of the compounds listed in Table 1-Table 6, which we predict to efficiently bind to PANDA Cysteines and efficiently rescue p53 in vitro, in vivo and/or in situ. In certain embodiments, the PANDA Agent is one or more of As₂O₃ (an FDA approved drug arsenic trioxide (“ATO”) for acute promyelocytic leukemia (“APL”)), As₂O₅, KAsO₂, NaAsO₂, HAsNa₂O₄, HAsK₂O₄, AsF₃, AsCl₃, AsBr₃, AsI₃, AsAc₃, As(OC₂H₅)₃, As(OCH₃)₃, As₂(SO₄)₃, (CH₃CO₂)₃As, C₈H₄K₂O₁₂As₂.xH₂O, HOC₆H₄COOAsO, [O₂CCH₂C(OH)(CO₂)CH₂CO₂]As, Sb₂O₃, Sb₂O₅, KSbO₂, NaSbO₂, HSbNa₂O₄, HSbK₂O₄, SbF₃, SbCl₃, SbBr₃, Sb₃, SbAc₃, Sb(OC₂H₅)₃, Sb(OCH₃)₃, Sb₂(SO₄)₃, (CH₃CO₂)₃Sb, C₈H₄K₂O₁₂Sb₂.xH₂O, HOC₆H₄COOSbO, [O₂CCH₂C(OH)(CO₂)CH₂CO₂]Sb, Bi₂O₃, Bi₂O₅, KBiO₂, NaBiO₂, HBiNa₂O₄, HBiK₂O₄, BiF₃, BiCl₃, BiBr₃, BiI₃, BiAc₃, Bi(OC₂H₅)₃, Bi(OCH₃)₃, Bi₂(SO₄)₃, (CH₃CO₂)₃Bi, C₈H₄K₂O₁₂Bi₂.xH₂O, HOC₆H₄COOBiO, C₁₆H₁₈As₂N₄O₂ (NSC92909), C₁₃H₁₄As₂O₆ (NSC48300), C₁₀H₁₃NO₈Sb (NSC31660), C₆H₁₂NaO₈Sb⁺ (NSC15609), C₁₃H₂₁NaO₉Sb⁺ (NSC15623), and/or combinations thereof. Further exemplar embodiments of PANDA Agent include those in Table 7, compounds that have strong p53 structural rescue capacity and p53 transcriptional activity (i.e. functional) rescue capacity, as confirmed by our experiments.

In certain embodiments, the PANDA Agent is not CP-31398; PRIMA-1; PRIMA-1-MET; SCH529074; Zinc; stictic acid, p53R3; methylene quinuclidinone; STIMA-1; 3-methylene-2-norbomanone; MIRA-1; MIRA-2; MIRA-3; NSC319725; NSC319726; SCH529074; PARP-PI3K; 5,50-(2,5-furandiyl)bis-2-thiophenemethanol; MPK-09; Zn-curc or curcumin-based Zn(II)-complex; P53R3; a (2-benzofuranyl)-quinazoline derivative; a nucleolipid derivative of 5-fluorouridine; a derivative of 2-aminoacetophenone hydrochloride; PK083; PK5174; PK7088; and other mp53 rescue compound previously identified by other groups.

A preferred mp53 has at least one mutation on p53, including any single amino acid mutation. Preferably, the mutation alters and/or partially alters the structure and/or function of p53, and more preferably the mutation is a rescuable mutation. Exemplary rescuable p53 mutations are listed in Table 8.

In certain preferred embodiments, as compared to when the PANDA Agent is not bound, the formed PANDA complex has gained one or more wtp53 structure, preferably a DNA binding structure; has gained one or more wtp53 function, preferably a transcription function; and/or has lost and/or diminishes one or more mp53 function, preferably an oncogenic function. The wildtype function can be gained in vitro and/or in vivo. Exemplary wildtype function gained can be at the molecule-level, such as association to nucleic acids, transcriptional activation or repression of target genes, association to wtp53 or mp53 partners, dissociation to wtp53 or mp53 partners, and reception to post-translational modification; at the cellular-level, such as, responsiveness to stresses such as nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, invasion and metastasis; and at the organism-level, such as delay or prevention of cancer relapse, increase of cancer treatment efficacy, increase of response ratio to cancer treatment, regulation of development, senescence, longevity, immunological processes, aging, combinations thereof, and the like. The mp53 functions can be lost, impaired and/or abrogated in vitro and/or in vivo. Exemplary mp53 function lost can include any functions, such as oncogenic functions, that promote cancer cell metastasis, genomic instability, invasion, migration, scattering, angiogenesis, stem cell expansion, survival, proliferation, tissue remodelling, resistance to therapy, mitogenic defects, combinations thereof and the like.

In certain preferred embodiments, the PANDA Agent can cause the mp53 to gain and/or lose the ability to upregulate or downregulate one or more p53 downstream targets, at an RNA level and/or protein level, in a biological system. The preferred functional change for a PANDA or a mp53 is at least to about 1.5 times, preferably to at least about 3 times, more preferably to at least about 5 times, more preferably to at least about 10 times, and further preferably to about 100 times.

In certain preferred embodiments, the PANDA Agent can be used to treat a p53 disorders in a subject with mp53 and/or without functional p53, preferably the mp53 is a rescuable mp53.

In certain preferred embodiments, PANDA Agent can suppress tumors, preferably least to a level that is statistically significant; more preferably having the ability to strongly suppress tumors at a level that is statistically significant. In certain preferred embodiments, the formed PANDA has the ability to regulate cell growth or tumor growth preferably to at least about 10% of the wtp53 level, further preferably at least about 100% of the wtp53 level, further preferably exceeding about 100% of the wtp53 level.

In certain preferred embodiments, the PANDA Agent can rescue one or more wtp53 structure, preferably a DNA binding structure; rescue one or more wtp53 function, preferably a transcription function; and eliminate and/or diminish one or more mp53 function, preferably an oncogenic function. In certain preferred embodiments, this is achieved by combining PANDA Agent with a p53 to form PANDA, preferably a mp53 with at least one mutation on p53, including a single amino acid mutation. Preferably, the mutation alters and/or partially alters the structure and/or function of p53. More preferably, the mutation is a rescuable p53 mutation. Exemplary rescuable p53 mutations are listed in Table 8.

In certain preferred embodiments, one or more wtp53 structure, preferably a DNA binding structure can be rescued by adding a PANDA and/or a PANDA Agent to a cell, preferably a human cell, and/or a subject, preferably a mammal, more preferably, further preferably a human.

In certain preferred embodiments, one or more wtp53 function, preferably a transcription function can be rescued by adding a PANDA and/or a PANDA Agent to a cell, preferably a human cell, and/or a subject, preferably a human subject. In certain preferred embodiments, one or more mp53 function, preferably an oncogenic function, can be eliminated and/or diminished by adding a PANDA and/or a PANDA Agent to a cell, preferably a human cell, and/or a subject, preferably a mammal, further preferably a human subject.

We disclose herein a method of using the PANDA or PANDA Agent in vitro and/or in vivo to rescue one or more wtp53 structure, preferably a DNA binding structure; rescue one or more wtp53 function, preferably a transcription function; eliminate and/or diminish one or more mp53 function, preferably an oncogenic function, the method comprising the step of adding an effective amount of PANDA or PANDA Agent to a cell, preferably a human cell, and/or subject, preferably a human subject.

The described PANDA Agent can be used to treat a p53 disorder in a subject with mp53, the disorder is preferably cancer and/or tumor.

In certain embodiments, the PANDA Agent can be formulated in a pharmaceutical composition suitable for treating a subject with a p53 disorder. A pharmaceutical composition will typically contain a pharmaceutically acceptable carrier. Although oral administration of a compound is the preferred route of administration, other means of administration such as nasal, topical or rectal administration, or by injection or inhalation, are also contemplated. Depending on the intended mode of administration, the pharmaceutical compositions can be in the form of solid, semi-solid, or liquid dosage forms, such as, for example, tablets, suppositories, pills, capsules, powders, liquids, suspensions, ointments, or lotions, preferably in unit dosage form suitable for single administration of a precise dosage. One skilled in this art may further formulate the compound in an appropriate manner, and in accordance with accepted practices, such as those disclosed in Remington's Pharmaceutical Sciences, Gennaro, Ed., Mack Publishing Co., Easton, Pa. 1990.

In certain embodiments, the PANDA Agent can be formulated in a pharmaceutically acceptable salt or solvate. The pharmaceutically acceptable salt can be an ionizable drug that has been combined with a counter-ion to form a neutral complex. Converting a drug into a salt through this process can increase its chemical stability, render the complex easier to administer, and allow manipulation of the agent's pharmacokinetic profile (Patel, et al., 2009).

In certain embodiments, the PANDA Agent and PANDA have the following features:
(1) the As atom of the PANDA Agent ATO binds directly to p53 to form PANDA, in a process that changes p53 structure, including folds the mp53;
(2) PANDA Agent mediated PANDA formation can take place both in vitro and in vivo, including in mammals such as mice and humans;
(3) PANDA is remarkably similar to wtp53 in both structure and function;
(4) PANDA Agent ATO folds the structure of Structural mp53s with a striking high efficiency so that the structure of PANDA is remarkably similar to that of wtp53;
(5) PANDA Agent ATO rescues the transcriptional activity of Structural mp53 through PANDA with a strikingly high efficiency;
(6) PANDA Agent ATO inhibits growth of mp53 expressing cells in vitro and in vivo through PANDA;
(7) mp53 expressing cells treated with PANDA Agent ATO or cells containing PANDA actively responds to DNA-damaging treatment;
(8) PANDA Agent ATO is highly effective and specific to a diverse number of mp53 and is an effective mp53 rescue agent;
(9) PANDA Agent ATO and PANDA can directly combat a wide range of cancers, including acute myeloid leukemia (“AML”) and/or myelodysplastic syndromes (“MDS”); and
(10) cancer patients, including patients with AML and MDS begin to show remarkable response to anti-cancer treatments when treated with ATO or PANDA.

Also described herein, are improved methods of diagnosing, prognosing, and treating a p53 disorder, such as cancer and methods of using the PANDA Agent, including in the diagnosis, prognosis, and treatment of a p53 disorder such as cancer are also described. The method comprises the step of administering to a subject an effective amount of a therapeutic, wherein the therapeutic comprises one or more PANDA Agent. In a preferred embodiment, the therapeutic is administered in combination with one or more additional therapeutics, preferably any known therapeutic effective at treating cancer and/or DNA damaging agent.

We further disclose a highly-efficient personalized method of treatment for a p53 disorder in a subject in need thereof. The method comprises the steps of:
(a) obtaining a sample from the subject;
(b) sequencing the TP53 in the sample;
(c) determining whether the TP53 and/or the corresponding p53 of the subject is rescuable;
(d) identifying one or more PANDA Agents and/or a combination of PANDA Agents that are most effective and/or appropriate to rescue the p53 in the subject; and
(e) administering an effective amount of the PANDA Agent and/or the combination of PANDA Agent to the subject;
wherein step (c) includes the step(s) (i) determining in silico whether the sequence of the TP53 DNA and/or the corresponding p53 is comparable to a database of rescuable p53s; and/or (ii) determining in vitro and/or in vivo whether the p53 of the subject can be rescued by screening it against a panel of PANDA Agents.

We further disclose a method of identifying PANDA. The method comprising the step of: using an antibody specific for properly folded PANDA, such as PAb1620, PAb246, and/or PAb240, to perform immunoprecipitation, wherein the immunoprecipitation is performed at a temperature of greater than 4° C.; measuring increase of molecular weight by mass spectroscopy; measuring whether transcriptional activity is rescued in a luciferase assay; measuring the mRNA and protein levels of p53 targets; measuring the p53-specific DNA binding ability; co-crystalizing to construct 3-D structure; and/or measuring increase of Tₘ.

We disclose herein a collection of PANDA Agents having the ability to regulate the levels of p53 targets in a biological system expressing a mp53 or lacking any functional p53. We further disclose a method of controlling one or more proteins and/or RNA regulated by p53 and/or PANDA, the method comprising the step of administering a regulator to a biological system, wherein the regulator is selected from the group consisting of:
(i) one or more PANDA Agent(s);
(ii) one or more PANDA(s);
(iii) one or more compound(s) that removes the PANDA Agent from the p53;
(iv) one or more mp53(s);
(v) one or more compound(s) that removes PANDA, including an anti-p53 antibody, a doxcycline, and anti-PANDA antibody; and
(vi) a combination thereof.

We disclose herein a collection of PANDA Agents having the ability to suppress tumors in a biological system, preferably a system that expresses a mp53. We further disclose a method of suppressing tumors, the method comprising the step(s) of administering to a subject in need thereof an effective amount of a therapeutic, where the therapeutic comprises a tumor suppressor selected from the group consisting of:
(i) one or more PANDA Agent(s); and
(ii) one or more PANDA(s).

In a preferred embodiment, the suppressor is administered in combination with one or more additional suppressors, preferably any known suppressor effective at suppressing tumor growth and/or DNA damaging agent.

We disclose herein a collection of PANDA Agents having the ability to regulate cell growth or tumor growth in a biological system, preferably a system that expresses a mp53. We further disclose a method of regulating cell growth or tumor growth, the method comprising the step of administering to a subject in need thereof an effective amount of a regulator, wherein the regulator is selected from the group consisting of:
(i) one or more PANDA Agent(s); and (ii) one or more PANDA. In a preferred embodiment, the regulator is administered in combination with one or more additional regulators, preferably any known regulator effective at slowing cell growth and/or DNA damaging agent.

We disclose herein a method of diagnosing a p53 disorder, such as cancer, tumor, aging, developmental diseases, accelerated aging, immunological diseases, combinations thereof and the like, in a subject in need thereof. The diagnosis method comprising the steps of administering to the subject an effective amount of a therapeutic, and detecting whether PANDA is formed wherein the therapeutic is selected from the group consisting of:
(i) one or more PANDA Agent(s); and
(ii) one or more PANDA(s).

In a preferred embodiment, the diagnosing method includes a treatment step wherein the therapeutic is administered in combination with one or more additional therapeutics, such as one or more additional PANDA Agent(s) and/or any other known therapeutic effective at treating cancer and/or DNA damaging agent, to effectively treat the p53 disorder in the subject.

In certain embodiments, the PANDA Agent has the potential to bind multiple cysteines and can selectively inhibit Structural mp53 expressing cells via promoting mp53 folding.

In certain embodiments, formed PANDA complex can be purified and isolated using any conventional methods, including any methods disclosed in this Application, such as by immunoprecipitation using PAb1620.

### BRIEF DESCRIPTION OF THE DRAWINGS

FIG. 1 shows p53 mutation hotspots. Top left panel shows p53 mutations with high frequency. Top right panel shows the 3D structure of the p53-DNA complex (PDB accession: 1TUP) generated by Pymol. mp53 function in contacting DNA are in gray solid spheres (R248 and R273). mp53 function in maintaining p53 structure are in black solid spheres (R175, G245, R249, and R282). C### designate the 10 p53 cysteines, which includes the 4 cysteine pairs: C176/C182, C238/C242, C135/C141, and C275/C277, and the PANDA Cysteines (C124, C135, and C141). Lower left panel, schematic of the six mp53 hotspots and DNA overlayed on a PANDA drawing. Lower right panel, schematic of PANDA illustrating the contacting residues R248 and R282 holding and eating the bamboo. PANDA Pocket is depicted as the hind neck known to stabilize a panda cub when being grabbed by its mother.

FIG. 2 shows TP53 is the most commonly mutated gene across cancer types and often within cancer types.

FIG. 3 shows Kaplan-Meier survival curves shows hazard ratio (HR) and P value (Log-rank test in univariate Cox proportional hazard model) in 18 large-scale TCGA cancer studies (8,810 patients). Of the overall 28 TCGA cancer studies with available patient overall survival data compiled from cBioPortal in November 2018, 10 studies (CESC, KIRC, KIRP, TGCT, THCA, THYM, ACC, CHOL, DLBC, and KICH) with either p53 mutation frequency <5% or patient number <100 were excluded from analysis. b, summarized p53 mutation hazard ratio for above 18 cohorts and 6 MDS/AML cohorts from literatures. Only cohorts with >5% p53 mutation frequency and >100 patients were compiled from literatures.

FIG. 4 shows clinical p53 mutations detected by Shanghai Institute of Hematology (SIH) and p53 mutations reported in AML/MDS patients.

FIG. 5 shows G150 growth inhibition plot graph (retrieved by CellMiner) of ATO, KAsO₂, Nutiin3, PRIMA-1, and NSC319726 in the NC160 cell panels shows ATO and KAsO₂ selectively targets Structural mp53s when it inhibits maligancies. p53 status was compiled via the IARC TP53 database. “Struc.” means cell lines expressing structural hotspot mp53 (R175, G245, R249, and R282); “WT” means cell lines expressing wtp53; “Others” means the remaining cell lines; “Null” means truncated p53, frame-shift p53 and null p53; “Contact” means hotspot mutations on R248 and R273; “*” means p<0.05.

FIG. 6 shows p53-R175H transfected H1299 cells or Trp53-R172H/R172H MEFs were treated with ATO or KAsO₂ for 2 hr, lysed, immunoprecipitated using PAb1620, PAb240, or PAb246 IP, and immunoblotted with p53 antibody.

FIG. 7 shows mass spectroscopy analysis of various mp53s in the presence and absence of ATO showing that the As atom bound to the mp53s.

FIG. 8 shows deconvoluted mass spectroscopy shows that molecular weights of purified recombinant mp53(94-293) core with an R249S mutation, increased, in the presence of As₂O₃, NaAsO₂, SbCl₃, and HOC₆H₄COOBiO, by approximately 72 Daltons (Da), 72 Da, 119 Da, and 206 Da, respectively, under native denaturing conditions. The increase roughly corresponds to a loss of 3 protons and a gain of an arsenic atom, arsenic atom, antimony atom and bismuth atom respectively. The purified mp53 core was incubated with 1.5 molar ratio of DMSO, As₂O₃, NaAsO₂, SbCl₃, or HOC₆H₄COOBiO overnight.

FIG. 9 shows melting temperature of various mp53s in the presence of various compounds. Melting curve of the purified recombinant p53C (p53C-WT, p53C-R175H, p53C-G245S, p53C-R249S and p53C-R282W, 5 μM for each reaction) were recorded via differential scanning fluorimetry (DSF) at the indicated ratio of ATO and other compounds. The apparent Tₘ of the p53C-R175H, p53C-G245S, p53C-R249S, and p53C-R282W can be raised by 1-8′C (mean±SD, n=3).

FIG. 10 shows the gene mutation frequency was derived from TCGA database by using cBioPortal.

FIG. 11. shows the p53-DNA complex (PDB accession: 1TUP) generated by Pymol. Left panel shows the 3 clusters of cysteines (C135/C141, C238/C242, C275/C277) and the R175-neighboring C176. Middle panel shows the PANDA complex purified from bacteria expressing p53(94-293)-R249S incubated with Asla (see also FIG. 13). Right panel shows the crystal of purified p53(94-293)-R249S soaked with 2 mM EDTA and 2 mM ATO for 19 h.

FIG. 12 shows PANDA Agent mediated functional and structural rescue. For p53 folding assay, H1299 cells transfected with indicated TP53 were treated with 1 μg/ml ATO for 2 hr, and cells were lysed followed by immunoprecipitation using PAb1620. Immunoprecipitated p53 was immunoblotted. Experiments are repeated twice. For p53 transcriptional activity assay, H1299 cells were co-transfected with indicated TP53 and PUMA reporter for 24 hr, followed by treatment of 1 μg/ml ATO for 24 hr. Plot shows the ATO-mediated mp53 rescue profile, derived from p53 folding assay and transcriptional activity assay. X-axis: PAb1620 IP efficiency; Y-axis: PUMA luciferase report signal. Hollow cycles: without ATO treatment; solid cycles: with ATO treatment.

FIG. 13 shows the 3D structure of p53. Upper panel shows the 3D structure of PANDA shown as ribbons. The PANDA Triad and arsenic atom are shown as spheres, the PANDA Pocket are shown in darker color. Middle panel shows the 3D structure of PANDA shown as spheres. The PANDA Pocket are shown in darker color. Lower panel shows the residues of PANDA Pocket. The structure are organized.

FIG. 14 Left panel shows H1299 cells were co-transfected with indicated TP53 mutation on p53-G245S plasmid and either PUMA reporter or PIG3 reporter for 24 hr. Bar graph shows the transcriptional activity of p53-G245S with designated SSSMs (mean±SD, n=3). Right panel, the upwards arrows and downwards arrows show the locations of mutations tested in left panel. Upwards arrows (S116 and Q136): mutations rescue p53-G245S, Downwards arrows: mutations fail to rescue p53-G245S.

FIG. 15 shows ATO efficiently and properly folds mp53s. Left panel, H1299 cells transfected with the p53-R175H DNA were treated with indicated agents for overnight, cells were lysed followed by PAb1620 IP. Right graph shows the normalized change of PAb1620 IP efficiency compared with the one in DMSO group. Numbers in the brackets followed agents indicate the concentration used (μg/ml).

FIG. 16 shows PANDA regains DNA-binding ability. H1299 cells expressing p53-R175H were treated with indicated agents overnight, and cells were lysed followed by pull-down assay using streptavidin beads in presence of 10 pM of biotinylated double-stranded DNA. p53-R175H was immunoblotted.

FIG. 17 shows PANDA regains wildtype-like transcriptional activity, which can be switched off by Dox. In upper left panel, H1299 cells expressing tet-off-regulated p53-R175H were pretreated with/without doxycycline (“Dox”) for 48 hr, followed by transfection of reporters containing the promoters of p53 targets in the presence/absence of 1 μg/ml ATO overnight. Bar graph shows mean±SD of luciferase signals from three independent experiments (n=3, **shows p<0.01). Lower left panel shows the rescued p53-R175H was largely depleted by DOX. Middle and right panel shows H1299 cells co-transfected with either p53-R282W DNA and reporters containing the promoters of PUMA or p53-G245S DNA and PIG3 reporter for 24 hr, followed by treatment of indicated agents for 24 hr. Numbers in the brackets indicate the concentration used (μg/ml). Bar graph shows normalized changes of transcriptional activity as indicated by luciferase signals (mean±SD, n=3).

FIG. 18 shows HCT116 cells transfected with indicated mp53s were treated with 1 μg/ml ATO for 48 hr. Protein levels of PUMA was determined.

FIG. 19 shows PANDA-R175H suppresses cell growth as shown in elevated sensitivity to cell death when ATO is added to H1299 cells expressing tet-off-regulated p53-R175H. Left panel shows MTT cell viability assay and right panel shows colony formation assay (mean±SD, n=3, *p<0.05). ATO was added for 48 hr and H1299 cells were pretreated with/without doxycycline (DOX) for 48 hr.

FIG. 20 shows PANDA-mediated tumor suppression includes malignancy inhibition. Cell viability (IC50) is for cells expressing Structural mp53s (R175 and R249) is lowered as compared to cells expressing wtp53 or null/truncated p53. Positive control Nutlin (a MDM2 inhibitor and thus a wtp53 reactivator), preferably targeted wtp53 in the cell lines. Cells were treated with ATO or Nutlin for 48 hr. Each value is a mean value of three independent experiments.

FIG. 21 shows PANDA-mediated tumor suppression. H1299 cells expressing tet-off-regulated p53-R175H were subcutaneously injected into flanks of nude mice. 5 mg/kg ATO was intraperitoneally injected for 6 consecutive d/week when the tumor area reached 0.1 cm (day 1). In DOX groups, drinking water contained 0.2 mg/ml DOX. Tumor size measurement was repeated every 3 day (left panel). Mice were sacrificed on day 28 and isolated tumors were weighed. Tumors size and weight were suppressed by over 90% according in ATO treated mice (left and lower right panel). Tumor suppression was predominantly PANDA-R175H-dependent, as shown by abrogation of ATO mediated tumor suppression after p53-R175H depletion by doxycycline (compare black solid line to black dot line for tumor size; compare last two bars for tumor weight). p53 IHC staining (right panel, bar=50 μm), H&E staining (data not shown), and p53 protein level measurement (data not shown) are also demonstrate ATO mediated tumor suppression. Graphs show mean±SEM (*p<0.05, **p<0.01, ***p<0.001, n=4/group).

FIG. 22 shows PANDA-mediated tumor suppression. CEM-C1 (hCD45+) cancer cells xenographed by tail vein injection into NOD/SCID mice can be detected on day 22 and reached to 0.1% in PB on day 23. Administering 5 mg/kg of ATO intravenously from day 23 onwards at 6 consecutive days per week significantly slowed the propagation of CEM-C1 cells in PB at day 26 and extended the survival of the injected mice (n=7) as compared to the control (Ctr vehicle, n=6). Samples were obtained from the mice retro-orbital sinus every 3 or 4 days from day 7 to day 26. Left panel, the percentage of mCD45+ and hCD45+ cells in PB on day 16, 22, and 26. Right panel, Mantel-Cox survival curves of vehicle or treated mice.

FIG. 23 shows MEFs expressing p53-R172H/R172H DNA or null p53 DNA were treated with ATO for 48 hr, followed by cell viability assay (left panel) and colony formation assay (right panel) (mean±SD, n=3, *p<0.05).

FIG. 24 shows cell viability assay showing ATO synergizes the effect of other clinical drugs such as the MDM2 inhibitor Nutlin3. H1299 cells cell viability assay of cells with null p53 DNA, p53-R175H DNA, or wtp53 DNA is treated with Nutin the absence or presence of 1 μg/ml ATO shows Nutlin dependent inhibition of only cells expressing wtp53 in the absence of ATO. However, in the presence of ATO, Nutlin dependent inhibition is also observed in cells expressing p53-R175. (mean±SD, n=3, *p<0.05).

FIG. 25 Top panel shows synergic effect of combinational treatment of ATO and the indicated chemotherapy agents (CIS: Cisplatin; ETO: Etoposide; ADM: Adriamycin (Doxorubicin); ARA: Cytarabine; AZA: Azacitidine; DAC: Decitabine.) in vitro. H1299 cells expressing tet-off-regulated p53-R175H were treated for 12 hr and the protein levels were measured. Middle panel shows synergistic effect of ATO and CIS, AZA, and DAC as measured in viability assay of Thp-1 cells transfected with p53-R282.

FIG. 26 shows clinical trial of ATO and DNA-damaging agents to treat AML/MDS. 50 MDS patients were recruited for p53 mutation-based personalized clinical trial.

FIG. 27 Heatmap shows significantly upregulated targets upon compound treatment. Upregulated targets are shown as grey bars while non-upregulated targets are shown as black bars.

FIG. 28 shows ATO is highly efficient and specific to a number of p53 with low off-target potential as shown in Thp-1 cells and U937 cells.

### DETAILED DESCRIPTION

### 1.1 Interpretations and Definitions

Unless otherwise indicated, this description employs conventional chemical, biochemical, molecular biology, genetics and pharmacology methods and terms that have their ordinary meaning to persons of skill in this field. All publications, references, patents and patent applications cited herein are hereby incorporated herein by reference in their entireties.

As used herein, the biological sample corresponds to any sample taken from a subject, and can include tissue samples and fluid samples such as blood, lymph or interstitial fluid and combinations thereof and the like.

As used in this specification and the appended claims, the following general rules apply. Singular forms “a,” “an” and “the” include plural references unless the content clearly indicates otherwise. General nomenclature rules for genes and proteins also apply. That is, genes are italicized or underlined (e.g.: TP53 or TP53), but gene products, such as proteins and peptides, are in standard font, not italicized or underlined (e.g.: p53). General rules for nomenclature of amino acid location also applies; that is, the amino acid abbreviation followed by number (e.g.: R175, R 175, R-175), where the amino acid name is represented by the abbreviation (e.g.: arginine by “R,” “arg,” “Arg” any other abbreviations familiar to those skilled in the art) and the location of the amino acid on the protein or peptide is represented by the number (e.g.: 175 for position 175). General rules for nomenclature of mutations also apply; for example, R175H, means arginine at location 175 is substituted by histidine. As another example mutation on p53 at location 175 from R to H can be represented by for example “p53-R175H” or “mp53-R175H.” Unless specified otherwise, any amino acid position corresponds to the amino acid location on a wildtype p53, preferably the human wtp53 isoform “a” listed in Table 14. General nomenclature rules for organism classification also apply. That is order, family, genus and species names are italicized.

As used herein, the following terms shall have the specified meaning. The term “about” takes on its plain and ordinary meaning of “approximately” as a person of skill in the art would understand, and generally plus or minus 20%, unless specified otherwise. The term “comprise,” “comprising,” “contain,” “containing,” “include,” “including,” “include but not limited to,” or “characterized by” is inclusive or open-ended and does not exclude additional, unrecited elements.

As used herein, the following terms shall have the specified meaning:

“expression” or “level of expression” means the level of mRNAs or proteins encoded by the referenced gene.

“PANDA” is abbreviated for 253 AND Agent complex, means a complex comprised of one or more p53s and one or more PANDA Agents.

“PANDA Agent” means a composition of matter capable of forming at least one tight association with the PANDA Pocket and has one or more useful characteristic(s). Exemplary PANDA Agent is listed in Table 1-Table 7.

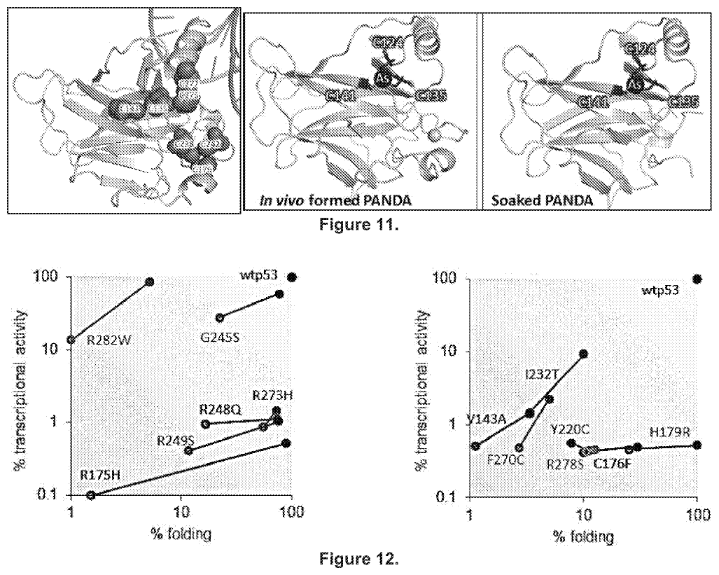

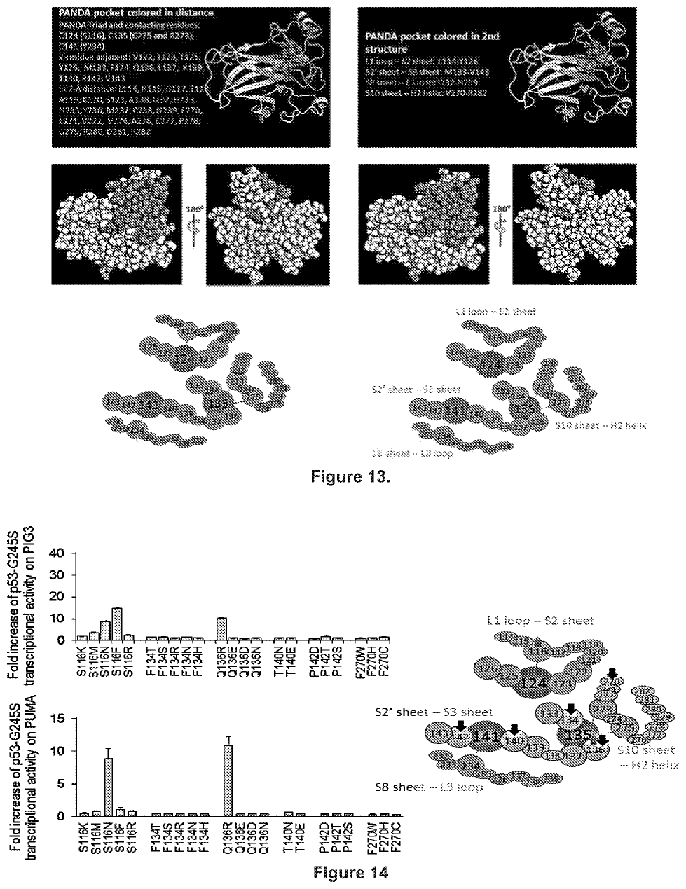

“PANDA Pocket” means a region consisting essentially of an area of about 7 Å from a properly folded PANDA Triad, including, all amino acids adjacent to one or more properly folded PANDA Triad, all amino acids that contact with one or more properly folded PANDA Triad, and all PANDA Triad. It is a pocket on p53 that interacts with one or more atoms of the PANDA Agent to form PANDA. Exemplary 3D structures of a PANDA Pockets can be found FIG. 11 and FIG. 13. In an exemplary embodiment, the PANDA Pocket can include all of the above amino acids, a subset of the above amino acids, and possibly other components as long as the resulting tertiary structure comprising the PANDA Pocket exhibits one or more of the useful characteristics described in this application. Thus, the PANDA Pocket can comprise or consist essentially of the above amino acids, or a subset thereof.

“PANDA Core” means the tertiary structure formed on the PANDA Pocket of a p53 when at least one tight association is formed between the PANDA Pocket and one or more atoms of the PANDA Agent.

“tight association” means a bond, covalent bond, a non-covalent bond (such as a hydrogen bond), and combinations thereof formed between PANDA Pocket and PANDA Agent. The tight association is preferably formed between a PANDA Agent and one or more PANDA Cysteines, preferably two or more PANDA Cysteines, and more preferably all three PANDA Cysteines.

“PANDA Cysteine” means a cysteine corresponding to one of the wtp53 positions at cysteine 124 (“C124” or “cys124”), cysteine 135 (“C135” or “cys135”), and cysteine 141 (“C141” or “cys141”) (together the “PANDA Triad”).

“p53” means any wildtype p53 (“wtp53”), including all natural and artificial p53; any mutated p53 (“mp53”), including all natural and artificial p53, combinations thereof, and the like.

“wtp53” means all wildtype p53 that is commonly considered as wildtype, or has a wildtype sequence, and includes any commonly acceptable variations, such as variations caused by single nucleotide polymorphism (“SNP”). Exemplary wtp53 includes p53α, p53β, p53γ, Δ40p53α, Δ40p53β, Δ40p53γ, and any acceptable variants, such as those with one or more single nucleotide polymorphisms (“SNP”). Exemplary wtp53 are listed in can be found in Table 14.

“SNP” means single-nucleotide polymorphism, which is a variation in a single nucleotide that occurs at a specific position in the genome, where each variation is presented to some appreciable degree within a population. An exemplary list of known SNP on p53 is Table 13.

“mp53” means mutated p53, which includes all p53 and p53 like macromolecules that is not a wtp53. mp53 includes, artificial mp53, such as recombinant p53, chimeric p53, p53 derivative, fusion p53, p53 fragment, and p53 peptide. Exemplary mp53 is a rescuable mp53.

“rescuable mp53” means a p53 with a rescuable mutation that can be rescued by a PANDA Agent (such as ATO), such that one or more of the mp53's wildtype function and/or structure can be rescued. A rescuable mp53 includes a structurally rescuable mp53 and a functionally rescuable mp53. Exemplary rescuable mp53s are provided in Table 8.

“structurally rescuable mp53” means a mp53 where one or more of the wild type structure can be rescued by a PANDA Agent (such as ATO).

“functionally rescuable mp53” means a mp53 where one or more of the wild type transcriptional function can be rescued by a PANDA Agent (such as ATO).

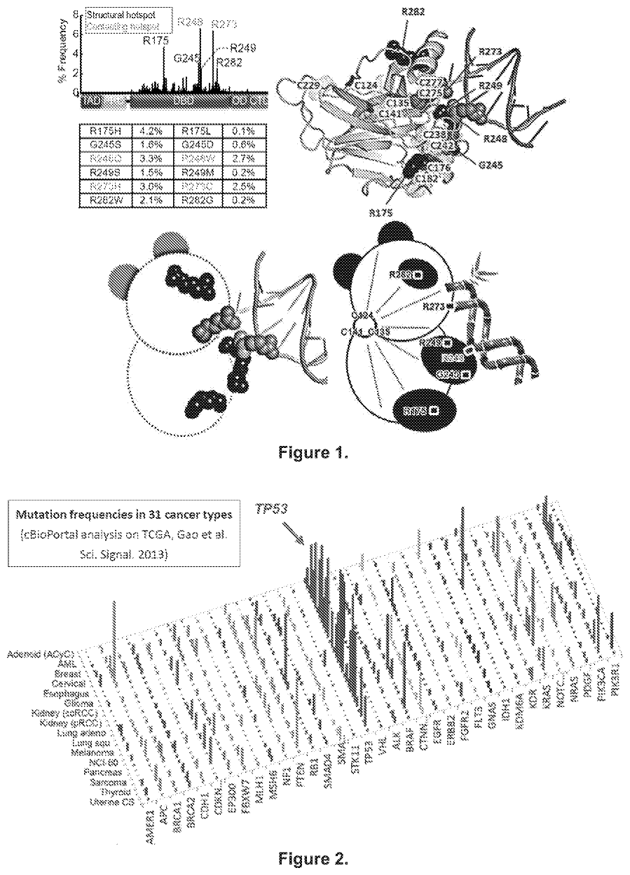

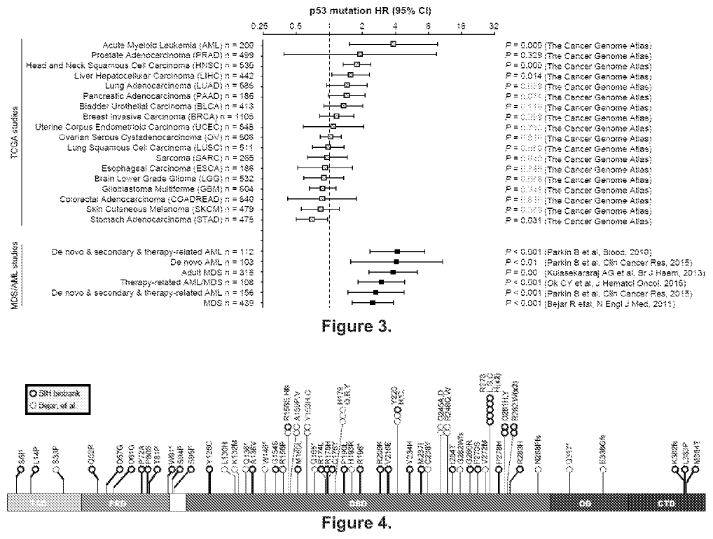

“hotspot mp53” means an mp53 with at least one mutation in mp53 hotspots, namely, R175, G245, R248, R249, R273, R282, combinations thereof, and the like. Examples of hotspot mp53s are listed in FIG. 1.

“Contacting mp53” means a mp53 that loses its DNA binding ability without drastically affecting the p53 structure. Contacting mp53s are represented by, for example, p53-R273H, p53-R273C, p53-R248Q and p53-R248W.

“Structural mp53” means a mp53 that has significantly disrupted three-dimensional structure as compared to wtp53. Structural mp53s are represented by, for example, p53-R175H, p53-G245D, p53-G245S, p53-R249S, and p53-R282W.

“artificial p53” means an artificially engineered p53. Preferred examples of an artificially engineered p53 include a p53 fusion protein, a p53 fragment, a p53 peptide, a p53-derived fusion macromolecule, a p53 recombinant protein, a p53 with second-site suppressor mutation (“SSSM”), and a super p53.

“p53 inhibiting protein” means a protein that inhibits a function of activity of p53, and includes, for example, murine double minute 2 (“MDM2”), inhibitor of apoptosis-stimulating protein of p53 (“iASPP”) and sirtuin-1 (“SIRT1”).

“useful characteristic” means an ability to efficiently and effectively rescue at least one wildtype structure, transcriptional activity, cell growth inhibition function, and/or tumor-suppressive function in a mp53. Exemplary useful characteristic includes: (a) an ability to substantially increase in the population of properly folded p53, preferably the increase is at least about 3 times more than the increase caused by PRIMA-1, more preferably the increase is at least about 5 times more than the increase caused by PRIMA-1, further preferably the increase is at least about 10 times more than the increase caused by PRIMA-1, further preferably the increase is at least about 100 times more than the increase caused by PRIMA-1; (b) an ability to substantially improve the transcription function of p53, preferably the improvement is at least about 3 times more than the improvement caused by PRIMA-1; more preferably the improvement is at least about 5 times more than the improvement caused by PRIMA-1, further preferably the improvement is at least about 10 times more than the improvement caused by PRIMA-1, further preferably the improvement is at least about 100 times than the improvement caused by PRIMA-1; and (c) an ability to substantially enhance the stability of p53 as measured by, for example, an increase p53 Tm, preferably the enhancement is at least about 3 times more than the enhancement caused by PRIMA-1, more preferably the improvement is at least about 5 times more than the improvement caused by PRIMA-1, further preferably the improvement is at least about 10 times more than the improvement caused by PRIMA-1, further preferably the improvement is at least about 100 times than the improvement caused by PRIMA-1. A preferred PANDA Agent has two or more useful characteristics, and more preferably has three or more useful characteristics. An exemplary PANDA Agent is ATO. Other exemplary PANDA Agent includes As analogs. Additional exemplary PANDA Agents are listed in Table 1-Table 7.

“efficiently” or “efficient” as used to describe the enhancement for a useful characteristic, rescue at least one wildtype structure, transcriptional activity, cell growth inhibition function, and/or tumor-suppressive function in a mp53, generally means enhancing the useful characteristic by more than about 3 times, as compared to the enhancement by PRIMA-1, preferably by more than about 5 times, more preferably by more than about 10 times, more preferably by about 100 times. For example, an efficient enhancement would be enhancing the Tₘ of mp53 by about 3-100 times of those of PRIMA-1, and/or folds mp53 by 3-100 times of those of PRIMA-1, and/or stimulates mp53's transcriptional activity by about 3-100 times of those of PRIMA-1.

“ATO” or “As₂O₃” means arsenic trioxide and compounds generally understood as arsenic trioxide.

“analog” or “analogue” means a compound obtained by varying the chemical structure of an original compound, for example, via a simple reaction or the substitution of an atom, moiety, or functional group of the original compound. Such analog may involve the insertion, deletion, or substitution of one or more atoms, moieties, or functional groups without fundamentally altering the essential scaffold of the original compound. Examples of such atoms, moieties, or functional groups include, but are not limited to, methyl, ethyl, propyl, butyl, hydroxyl, ester, ether, acyl, alkyl, carboxyl, halide, ketyl, carbonyl, aldehyde, alkenyl, azide, benzyl, fluoro, formyl, amide, imide, phenyl, nitrile, methoxy, phosphate, phosphodiester, vinyl, thiol, sulfide, or sulfoxide atoms, moieties, or functional groups. Many methods for creating a chemical analog from an original compound are known in the art.

“p53 disorder” means an abnormal physical and/or mental condition caused by a mutation in the TP53 gene and/or p53 protein. The condition can be in a human or another animal, such as a mouse, dog and other companion animals, a cattle and other livestock, a wolf or other zoo animals, and a horse or other equines. Examples of a p53 disorder include cancer, such as carcinoma (for example adenocarcinomas and squamous cell carcinoma), sarcoma, myeloma, leukemia, lymphoma, blastoma, and mixed types cancers (for example, adenosquamous carcinoma, mixed mesodermal tumor, carcinosarcoma, and teratocarcinoma); a tumor (for example, a tumor in connective tissue, endothelium and mesothelium, blood and lymphoid cells, muscle, epithelial tissues, neural, amine precursor uptake and decarboxylation system, other neural crest-derived cells, breast, renal anlage, and/or gonadal); a neurological disease, a developmental disease, an immunological disease, and aging, among others. Additional examples of known p53 disorder are listed in Section 1.2. A p53 cancer and/or tumor is a cancer and/or tumor with at least one p53 mutation. Additional examples of known p53 cancer and/or tumor are listed in Section 1.3.

“subject” means any organism. The subject is preferably an animal, such as a vertebrate; further preferably a mammal, such as a cattle, a horse, a pig, a lamb, and other livestock; further preferably a human, such as a patient, a cancer patient, an unborn child, and any un-conceived, hypothetical child of two parents.

“a person in need of” means an individual who has a p53 disorder, such as a cancer, wherein the cancer expresses a mp53, preferably a rescuable mp53.

“biological system” means a cell, bacteria, artificial system containing p53 pathway and relevant proteins.

“treatment” means the administration and/or application of the therapeutic product or method to a subject with a p53 disorder, and includes, among others, monitoring the efficacy of a type of treatment for the p53 disorder.

“diagnosis” means any method to identify a particular disease, and includes, among others, detecting the symptoms of a disease, assessing the severity of the disease, determining the stages of the disease, and monitoring the progression of the disease.

“prognosis” means any method to determine the likely course of a disease, and includes, among others, determining the predisposition of a disease, determining the likelihood a disease will onset, assessing the likely severity of the disease, determining the likely stages of the disease, and predicting the likely progression of the disease.

“a therapeutically effective amount” is an amount of a compound effective to prevent, alleviate, or ameliorate symptoms of a disorder or prolong the survival of the subject being treated. Determination of a therapeutically effective amount is well within the capability of those skilled in the art, especially in light of the detailed disclosure provided herein. The effective dosage, level, or amount of a compound to be used in vivo can be determined by those skilled in the art, taking into account the disorder to be treated, the condition of the individual patient, the site of delivery, the method of administration, the potency, bioavailability, and metabolic characteristics of the compound, and other factors.

“screening of effective treatments” means screening of effective therapeutic product or method for the treatment of a certain disease. It can involve in vitro and/or ex vivo screening methods, and includes, among others, both the product or composition to treat a disease and the method to prepare the composition for treatment.

“carrier” as used herein can include solvents, dispersion media, vehicles, coatings, diluents, antibacterial and antifungal agents, isotonic and absorption delaying agents, buffers, carrier solutions, suspensions, colloids, and the like

“pharmaceutical carrier” as used herein can include, liposomes, albumin microspheres, soluble synthetic polymers, DNA complexes, protein-drug conjugates, carrier erythrocytes, and any other substance that is incorporated to improve the delivery and the effectiveness of drugs. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

“compatible therapy for p53 disorder” means a therapy (including experimental therapies) compatible and/or synergistic with p53 treatments containing one or more PANDA Agents, The compatible therapy for p53 disorder can include surgery, chemotherapy, and radiation therapy. Experimental therapies include, but are not limited to, expression of wtp53 in tumors based on viral or viral like particle based delivery vectors.

“p53 cancer therapeutic” as used herein include, general chemotherapeutics. Examples of general chemotherapeutics include, but are not limited to, Avastin, Rituxan, Herceptin, Taxol, and Gleevec.

“DTP” means Developmental Therapeutics Program as understood by a person of ordinary skill in the art.

“DNA damaging agents” mean the anti-cancer agents in which the DNA damaging is involved when they function. Examples of a DNA damaging agent include decitabine (“DAC”), clsplatin (“CIS”), etoposide (“ETO”), adriamycin (ADM”), 5-fluorouracil (“5-FU”), cytarabine (“ARA/araC”), and azactidine (“AZA”).

### 1.2p53 is One of the Most Important Proteins in Cell Biology

The 53-kilodalton p53 protein is a transcription factor and one of the most important proteins in cell biology. p53 is the most heavily studied protein in history and it is also the most heavily studied protein in every year since 2001, yet the reusability of mp53 is still largely unknown. Wildtype p53 (“wtp53”) sequence can be found in public gene banks, such as gene bank, protein bank, and Uniport. Exemplary wtp53 sequences are listed under Table 14. Unless specified otherwise, this application uses the wtp53 sequences of human p53 isoform “a” listed under Table 14 to reference amino acid locations on p53.

The active human wtp53 is a homotetramer of 4×393 amino acids with multiple domains including an intrinsically disordered N-terminal transactivation domain (“TAD”), a proline-rich domain (“PRD”), a structured DNA-binding domain (“DBD”) and tetramerization domain (“TET”) connected via a flexible linker, and an intrinsically disordered C-terminal regulatory domain (“CTD”) (see FIG. 1). Many TP53 family genes expressing multiple isoforms exist, and often exhibit antagonistic functions.

wtp53 plays a central part in the cells and is frequently considered as the most important tumor suppressor. Upon cellular stresses, such as DNA damage or oncogenic stress, p53 is activated and transcriptionally regulates a batch of genes to trigger events including cell-cycle arrest, DNA repair, apoptosis, cell repair, cell death, among others. Examples of genes transcriptionally regulated by p53 include Apaf1, Bax, Fas, Dr5, mir-34, Noxa, TP53AIP1, Perp, Pidd, Pig3, Puma, Siva, YWHAZ, Btg2, Cdkn1a, Mdm2, BBC3/PUMA, Tp53i3, Gadd45a, mir-34a, mir-34b/34c, Prl3, Ptprv, Reprimo, Pai1, Pml, Ddb2, Ercc5, Fancc, Gadd45a, Ku86, Mgmt, Mlh1, Msh2, P53r2, Polk, Xpc, Adora2b, Aldh4, Gamt, Gls2, Gpx1, Lpin1, Parkin, Prkab1, Prkab2, Pten, Sco1, Sesn1, Sesn2, Tigar, Tp53inp1, Tsc2, Atg10, Atg2b, Atg4a, Atg4c, Atg7, Ctsd, Ddit4, Dram1, Foxo3, Laptm4a, Lkb1, Pik3r3, Prkag2, Puma, Tpp1, Tsc2, Ulk1, Ulk2, Uvrag, Vamp4, Vmp1, Bai1, Cx3cl1, Icam1, Irf5, Irf9, Isg15, Maspin, Mcp1, Ncf2, Pai1, Tlr1-Tlr10, Tsp1, Ulbp1, Ulbp2, mir-34a, mir-200c, mir-145, mir-34a, mir-34b/34c, Notch1, combinations thereof and the like. In addition to anti-cancer role, p53 target genes also have important roles in senescence, angiogenesis, and autophagy, connecting, regulating oxidative stress, regulating metabolic homeostasis, stem cell maintenance, among others. Accordingly, a mutation in p53 (i.e. a mutant p53 or mp53) can cause a wide range of health issues, including cancer, tumor, neurological disease, developmental disease, immunological disease, and aging, among others.

Examples of known p53 disorders include achalasia, acinar cell carcinoma, acrofacial dysostosis, actinic cheilitis, actinic keratosis, acute lymphocytic leukemia, adenocarcinoma, adenoid cystic carcinoma, adenoma, adenosarcoma, adenosquamous carcinoma, adrenocortical carcinoma, adult hepatocellular carcinoma, adult medulloblastoma, adult t-cell leukemia, aging, agraphia, alpha-thalassemia, alpha-thalassemia/mental retardation syndrome, anal squamous cell carcinoma, anaplastic thyroid cancer, anogenital venereal wart, anterior cranial fossa meningioma, aplastic anemia, ataxia-telangiectasia, atrophic gastritis, atrophy of prostate, atypical follicular adenoma, atypical teratoid rhabdoid tumor, autonomic nervous system neoplasm, autosomal genetic disease, b cell prolymphocytic leukemia, Barrett esophagus, Barrett's adenocarcinoma, Bartholin's duct cyst, Bartholin's gland adenoma, Bartholin's gland benign neoplasm, basal cell carcinoma, basal cell carcinoma, basaloid squamous cell carcinoma, B-cell lymphomas, Beckwith-wiedemann syndrome, bile duct adenocarcinoma, bile duct carcinoma, biliary papillomatosis, biliary tract neoplasm, bladder cancer, bladder carcinoma in situ, bladder papillary transitional cell neoplasm, bladder squamous cell carcinoma, bladder transitional cell papilloma, bladder urothelial carcinoma, bone giant cell sarcoma, bone squamous cell carcinoma, brain cancer, brain ependymoma, brain glioblastoma multiforme, brain glioma, brain stem astrocytic neoplasm, brain stem cancer, brain stem glioma, breast adenocarcinoma, breast benign neoplasm, breast cancer, breast carcinoma in situ, breast disease, breast ductal carcinoma, breast malignant phyllodes tumor, breast squamous cell carcinoma, calcifying epithelial odontogenic tumor, cataract, cell type benign neoplasm, cell type cancer, cellular ependymoma, cellular neurofibroma, cellular schwannoma, central nervous system lymphoma, central nervous system organ benign neoplasm, central nervous system primitive neuroectodermal neoplasm, cerebellar angioblastoma, cerebellar astrocytoma, cerebellar liponeurocytoma, cerebellum cancer, cerebral convexity meningioma, cerebral neuroblastoma, cerebral primitive neuroectodermal tumor, cerebral ventricle cancer, cerebrum cancer, cervical adenocarcinoma, cervical cancer, cervical carcinosarcoma, cervical squamous cell carcinoma, cervix carcinoma, cervix small cell carcinoma, cervix uteri carcinoma in situ, cheilitis, childhood leukemia, cholangiocarcinoma, cholecystitis, chordoid glioma, chordoma, choroid plexus cancer, chromophobe adenoma, chronic salpingitis, clear cell adenocarcinoma, clear cell cystadenofibroma, clear cell ependymoma, clivus meningioma, cl/sll, colorectal adenocarcinoma, colorectal adenoma, colorectal cancer, conjunctival degeneration, conjunctival squamous cell carcinoma, connective tissue cancer, cystadenocarcinoma, cystic teratoma, cystitis, dedifferentiated liposarcoma, dermatofibrosarcoma protuberans, differentiated thyroid carcinoma, diffuse large B-cell lymphoma, ductal carcinoma in situ, dyskeratosis congenita autosomal recessive, dyskeratosis congenita, dyskeratosis congenita, autosomal recessive, eccrine sweat gland neoplasm, ectrodactyly, ectodermal dysplasia, and cleft lip/palate syndrome, embryonal sarcoma, endocervical adenocarcinoma, endocrine gland cancer, endometrial adenocarcinoma, endometrial cancer, endometrial clear cell adenocarcinoma, endometrial stromal sarcoma, endometrium carcinoma in situ, ependymoblastoma, epidermal appendage tumor, epidural neoplasm, epithelial-myoepithelial carcinoma, esophageal basaloid squamous cell carcinoma, esophageal cancer, esophageal disease, esophagitis, esophagus adenocarcinoma, essential thrombocythemia, estrogen-receptor positive breast cancer, Ewing sarcoma, fallopian tube adenocarcinoma, fallopian tube carcinoma, familial adenomatous polyposis, familial colorectal cancer, female breast cancer, female reproductive endometrioid cancer, female reproductive organ cancer, fibrillary astrocytoma, focal cortical dysplasia, type ii, frontal convexity meningioma, gallbladder cancer, gallbladder squamous cell carcinoma, ganglioglioma, gastric adenocarcinoma, gastric adenosquamous carcinoma, gastric cancer, gastric lymphoma, gastric papillary adenocarcinoma, gastroesophageal reflux, gastrointestinal stromal tumor, gastrointestinal system benign neoplasm, gastrointestinal system cancer, germ cell and embryonal cancer, giant cell glioblastoma, glioblastoma multiforme, glioblastoma, gliofibroma, glioma susceptibility, glioma, gliomatosis cerebri, gliosarcoma, glomangiosarcoma, glomus tumor, glycogen-rich clear cell breast carcinoma, grade iii astrocytoma, granulosa cell tumor of the ovary, Helicobacter pylori infection, hematologic cancer, hepadnavirus infection, hepatoblastoma, hepatocellular carcinoma, hereditary breast ovarian cancer syndrome, hidradenocarcinoma, histiocytoma, huntington disease, hydrocephalus, hyperplastic polyposis syndrome, hypoxia, in situ carcinoma, inflammatory myofibroblastic tumor, infratentorial cancer, integumentary system cancer, intestinal benign neoplasm, intestinal disease, intracranial chondrosarcoma, intrahepatic cholangiocarcinoma, invasive bladder transitional cell carcinoma, inverted papilloma, juvenile pilocytic astrocytoma, kaposi sarcoma, keratinizing squamous cell carcinoma, keratoacanthoma, keratocystic odontogenic tumor, larynx cancer, larynx verrucous carcinoma, leiomyosarcoma, leukemia, leukemia, acute lymphoblastic, leukemia, acute myeloid, leukemia, chronic lymphocytic, lichen disease, lichen planus, lichen sclerosus, li-fraumeni syndrome, li-fraumeni syndrome, lip cancer, liposarcoma, liver angiosarcoma, lung benign neoplasm, lung cancer susceptibility, lung cancer, lung occult squamous cell carcinoma, lung papillary adenocarcinoma, lung squamous cell carcinoma, lymph node cancer, lymphoid interstitial pneumonia, lymphoma, non-hodgkin, familial, lynch syndrome, male reproductive organ cancer, malignant ependymoma, malignant giant cell tumor, malignant mesenchymoma, malignant ovarian surface epithelial-stromal neoplasm, malignant peripheral nerve sheath tumor, malignant spiradenoma, mantle cell lymphoma, Marek disease, mature B-cell neoplasm, mature teratoma, maxillary sinus squamous cell carcinoma, medulloblastoma, medullomyoblastoma, megaesophagus, megakaryocytic leukemia, melanoma, melanoma, cutaneous malignant, meningeal melanomatosis, meninges sarcoma, meningioma, familial, merkel cell carcinoma, microglandular adenosis, mixed astrocytoma-ependymoma, mixed cell type cancer, mixed glioma, mixed oligodendroglioma-astrocytoma, mucoepidermoid esophageal carcinoma, multifocal osteogenic sarcoma, multiple cranial nerve palsy, muscle cancer, mutagen sensitivity, mutyh-associated polyposis, myasthenic syndrome, myelodysplastic syndrome, myeloid leukemia, myeloma, multiple, myxoid liposarcoma, myxosarcoma, nasal cavity adenocarcinoma, nasopharyngeal carcinoma, necrotizing sialometaplasia, nervous system cancer, neuroblastoma, nevus of ota, nijmegen breakage syndrome, non-invasive bladder papillary urothelial neoplasm, non-proliferative fibrocystic change of the breast, ocular cancer, olfactory groove meningioma, oligodendroglioma, optic nerve glioma, optic nerve neoplasm, oral cancer, oral cavity cancer, oral leukoplakia, organ system benign neoplasm, oropharynx cancer, osteogenic sarcoma, ovarian cancer, ovarian cancer, ovarian clear cell carcinoma, ovarian serous cystadenocarcinoma, ovary adenocarcinoma, ovary epithelial cancer, pancreas adenocarcinoma, pancreatic cancer, pancreatic ductal carcinoma, papillary adenocarcinoma, papillary serous adenocarcinoma, papilledema, papilloma of choroid plexus, papilloma, parameningeal embryonal rhabdomyosarcoma, parietal lobe neoplasm, penile cancer, penis carcinoma in situ, penis squamous cell carcinoma, periosteal osteogenic sarcoma, peripheral nervous system neoplasm, peripheral T-cell lymphoma, Peutz-jeghers syndrome, pharynx cancer, pigmented villonodular synovitis, pilocytic astrocytoma, pinguecula, plantar wart, pleomorphic adenoma carcinoma, pleomorphic adenoma, pleomorphic carcinoma, pleomorphic xanthoastrocytoma, pleuropulmonary blastoma, pre-malignant neoplasm, primary peritoneal carcinoma, prolactin producing pituitary tumor, prostate cancer, prostate squamous cell carcinoma, protoplasmic astrocytoma, pseudomyxoma peritonei, pulmonary blastoma, rare adenocarcinoma of the breast, recessive dystrophic epidermolysis bullosa, rectal neoplasm, papillary, renal cell carcinoma, respiratory system cancer, retinal cancer, retinoblastoma, rhabdomyosarcoma, Richter's syndrome, rift valley fever, ring chromosome, sarcoma, sarcomatoid squamous cell skin carcinoma, schneiderian carcinoma, sclerosing liposarcoma, scrotal carcinoma, sensory system cancer, serous cystadenocarcinoma, short-rib thoracic dysplasia with or without polydactyly, signet ring cell adenocarcinoma, skin melanoma, skin squamous cell carcinoma, small cell cancer of the lung, small cell carcinoma, small cell sarcoma, soft tissue sarcoma, spinal cancer, spinal cord astrocytoma, spinal cord glioma, spinal cord primitive neuroectodermal neoplasm, spiradenoma, spitz nevus, splenic diffuse red pulp small B-cell lymphoma, split-hand/foot malformation, sporadic breast cancer, squamous cell carcinoma, squamous cell papilloma, submandibular gland cancer, suppression of tumorigenicity, suppressor of tumorigenicity, supratentorial cancer, sweat gland cancer, synchronous bilateral breast carcinoma, teratoma, testicular germ cell tumor, testicular torsion, tetraploidy, thoracic benign neoplasm, thymus cancer, thyroid cancer, thyroid lymphoma, tongue cancer, tongue squamous cell carcinoma, transitional cell carcinoma, ulcerative stomatitis, ureteral obstruction, urinary tract papillary transitional cell benign neoplasm, uterine body mixed cancer, uterine carcinosarcoma, uterine corpus cancer, uterine corpus serous adenocarcinoma, vaccinia, vestibular gland benign neoplasm, vulva cancer, vulva squamous cell carcinoma, vulvar adenocarcinoma, vulvar intraepithelial neoplasia, vulvar sebaceous carcinoma, wilms tumor, xanthogranulomatous cholecystitis, xeroderma pigmentosum, variant type, zika virus infection, combinations thereof and the like.

It has been estimated that the direct medical expenses for mp53 patients in 2017 alone amounts to approximately 65 billion USD.

### 1.3p53 and Cancer

p53 is the most frequently mutated cancer protein (FIG. 2). A p53 mutation can eliminate the tumor suppressive function of wtp53. Additionally, a p53 mutation can gain oncogenic properties. For example, a mutant p53 (“mp53”) can promote cancer metastasis, confer resistance to treatment, and cause cancer patients to relapse. Accordingly, it is estimated that nearly half of all human cancers has mutated and inactivated p53 gene and/or protein (Vogelstein et al., 2000).

Examples of cancers and/or tumors reported to harbor one or more p53 mutations include carcinoma, acinar cell carcinoma, adenocarcinoma, adenoid cystic carcinoma, adenosquamous carcinoma, apocrine adenocarcinoma, basal cell carcinoma, basaloid carcinoma, basosquamous carcinoma, bronchiolo-alveolar adenocarcinoma, carcinoma in pleomorphic adenoma, cholangiocarcinoma, choriocarcinoma, choroid plexus carcinoma, clear cell adenocarcinoma, combined hepatocellular carcinoma and cholangiocarcinoma, comedocarcinoma, cribriform carcinoma, ductal carcinoma, solid type, eccrine adenocarcinoma, endometrioid adenocarcinoma, follicular adenocarcinoma, giant cell and spindle cell carcinoma, giant cell carcinoma, hepatocellular carcinoma, hepatoid adecarcinoma, infiltrating basal cell carcinoma, infiltrating duct carcinoma, infiltrating ductular carcinoma, inflammatory carcinoma, intraductal carcinoma, intraductal carcinoma and lobular carcinoma, intraductal papillary adenocarcinoma, intraductal papillary-mucinous carcinoma, large cell carcinoma, large cell neuroendocrine carcinoma, leiomyosarcoma, lobular carcinoma, medullary carcinoma, merkel cell carcinoma, metaplastic carcinoma, mixed cell adenocarcinoma, mucinous adenocarcinoma, mucinous cystadenocarcinoma, mucoepidermoid carcinoma, multifocal superficial basal cell carcinoma, neuroendocrine carcinoma, non-small cell carcinoma, oat cell carcinoma, papillary adenocarcinoma, papillary carcinoma, papillary cystadenocarcinoma, papillary serous cystadenocarcinoma, papillary transitional cell carcinoma, pituitary carcinoma, plasmacytoid carcinoma, pleomorphic carcinoma, pseudosarcomatous carcinoma, renal cell carcinoma, sebaceous adenocarcinoma, secretory carcinoma of breast, serous cystadenocarcinoma, serous surface papillary carcinoma, signet ring cell carcinoma, small cell carcinoma, solid carcinoma, spindle cell carcinoma, squamous cell carcinoma, sweat gland adenocarcinoma, teratocarcinoma, thymic carcinoma, transitional cell carcinoma, trichilemmocarcinoma, tubular adenocarcinoma, sarcoma, alveolar rhabdomyosarcoma, carcinosarcoma, chondroblastic osteosarcoma, chondrosarcoma, clear cell sarcoma of kidney, dedifferentiated chondrosarcoma, dermatofibrosarcoma, embryonal rhabdomyosarcoma, embryonal sarcoma, Ewing sarcoma, fibrosarcoma, gastrointestinal stromal sarcoma, gliosarcoma, hemangiosarcoma, kaposi sarcoma, liposarcoma, mixed liposarcoma, myxoid liposarcoma, osteosarcoma, periosteal osteosarcoma, pleomorphic liposarcoma, pleomorphic rhabdomyosarcoma, rhabdomyosarcoma, sarcoma, synovial sarcoma, undifferentiated sarcoma, myeloma, multiple myeloma, leukemia, acute leukemia, acute megakaryoblastic leukemia, acute monocytic leukemia, acute myeloid leukemia, acute myeloid leukemia, adult T-cell leukemia/lymphoma, aggressive NK-cell leukemia, B-cell chronic lymphocytic leukemia/small lymphocytic lymphoma, Burkitt cell leukemia, chronic myeloid leukemia, chronic myelomonocytic leukemia, hairy cell leukemia, lymphoid leukemia, myeloid leukemia, plasma cell leukemia, precursor B-cell lymphoblastic leukemia, precursor cell lymphoblastic leukemia, precursor T-cell lymphoblastic leukemia, prolymphocytic leukemia, T-cell large granular lymphocytic leukemia, undifferentiated leukemia, lymphoma, anaplastic large cell lymphoma, angioimmunoblastic T-cell lymphoma, Burkitt lymphoma, cutaneous T-cell lymphoma, follicular lymphoma, Hodgkin lymphoma, malignant lymphoma, mantle cell lymphoma, marginal zone B-cell lymphoma, mature T-cell lymphoma, NK/T-cell lymphoma, precursor cell lymphoblastic lymphoma, primary effusion lymphoma, splenic marginal zone-B-cell lymphoma, ameloblastoma, giant cell glioblastoma, glioblastoma, hepatoblastoma, medulloblastoma, nephroblastoma, neuroblastoma, pleuropulmonary blastoma, and pulmonary blastoma, and retinoblastoma, tumor, adenocarcinoid tumor, atypical carcinoid tumor, Brenner tumor, carcinoid tumor, epithelial tumor, gastrointestinal stromal tumor, giant cell tumor of soft parts, glomus tumor, granulosa cell tumor, Klatskin tumor, malignant peripheral nerve sheath tumor, malignant rhabdoid tumor, mesodermal mixed tumor, mixed tumor, mucinous cystic tumor of borderline malignancy, Mullerian mixed tumor, myofibroblastic tumor, peripheral neuroectodermal tumor, phyllodes tumor, phyllodes tumor, primitive neuroectodermal tumor, serous surface papillary tumor, solitary fibrous tumor, tumor cells, yolk sac tumor, adenoma, adrenal cortical adenoma, atypical adenoma, cystadenoma, atypical adenoma, cystadenoma, fibroadenoma, follicular adenoma, hepatocellular adenoma, intraductal papillary-mucinous adenoma, pleomorphic adenoma, serous cystadenoma, serrated adenoma, tubular adenoma, tubulovillous adenoma, villous adenoma, mixed tumors, angiomyolipoma, astrocytoma, atypical fibrous histiocytoma, Barrett's esophagus, Bowen disease, central neurocytoma, clear cell adenocarcinofibroma, dysgerminoma, dysplasia, embryo fibroblasts, endometriosis, ependymoma, esophagitis, essential thrombocythemia, fibrillary astrocytoma, fibrosis, gemistocytic astrocytoma, germinoma, glandular intraepithelial neoplasia, glioma, gliomatosis cerebri, glucagonoma, goblet cell carcinoid, hemangioendothelioma, hemangiopericytoma, hidrocystoma, hydatidiform mole, hyperplasia, insulinoma, keloid, keratoacanthoma, keratosis, Langerhans cell histiocytosis, lentigo maligna melanoma, leucoplakia, lipoma, malignant fibrous histiocytoma, malignant histiocytosis, malignant melanoma, malignant myoepithelioma, meningioma, mesothelioma, metaplasia, mixed glioma, mycosis fungoides, myelodysplastic syndrome, myelosclerosis with myeloid metaplasia, myoepithelioma, neoplasm, neurilemoma, nodular melanoma, oligodendroglioma, osteochondroma, pheochromocytoma, pigmented nevus, pilocytic astrocytoma, plasmacytoma, pleomorphic xanthoastrocytoma, polycythemia vera, polyp, pterygium, pulmonary sclerosing hemangioma, refractory anemia, seminoma, serous adenocarcinofibroma, Sezary syndrome, squamous intraepithelial neoplasia, superficial spreading melanoma, teratoma, thymoma, urothelial papilloma, Waldenstrom macroglobulinemia, aggressive fibromatosis, lymphomatoid papulosis, combinations thereof and the like.

### 1.4 Rescuing mp53

Approximately one-third of the p53 mutations are located on one of six mp53 hotspots: R175, G245, R248, R249, R273, and R282, (each a “mp53 hotspot”) (Freed-Pastor and Prives, 2012). Mutated p53 (or mp53) falls roughly into two categories. Contacting mp53 has lost its DNA binding ability without drastically affecting the p53 structure (“Contacting mp53”). Examples of Contacting mp53s include p53-R273H (3.0% mutation frequency), p53-R273C (2.5% mutation frequency), p53-R248Q (3.3% mutation frequency) and p53-R248W (2.7% mutation frequency). See also FIG. 1. Structural mp53 has lost its wtp53 3D structure (“Structural mp53”). Because Structural mp53 has lower thermal stability than wtp53, Structural mp53 has a much higher population of unfolded p53s than wtp53. Examples of Structural mp53s include p53-R175H (4.2% mutation frequency), p53-R175L (0.1% mutation frequency), p53-G245D (0.6% mutation frequency), p53-G245S (1.6% mutation frequency), p53-R249S (1.5% mutation frequency), p53-R249M (0.2% mutation frequency), p53-R282W (2.1% mutation frequency), and p53-R282G (0.2% mutation frequency). See also FIG. 1. Both Contacting mp53s and Structural mp53s has greatly impaired DNA-binding ability and transcriptional activity. Moreover, most of cancer-derived mp53s lose wtp53's tumor-suppressive functions and many also gain oncogenic properties.

As seen in its representative member, the R282W mutation disrupts the hydrogen-bond network in the local loop-sheet-helix motif, reducing the melting temperature (“T”, an index for thermally stability of protein) and cause global, structural destabilization. A broad-spectrum rescue agent would thus need to increase the Tₘ. We further discovered that four pairs of the 10 mp53 cysteines (C176/C182, C238/C242, C135/C141, and C275/C277) are in close proximity to the Structural mp53 hotspots (FIG. 11) and that covalently crosslinking the cysteine pairs and/or clusters can immobilize the local region and thereafter be enough to off-set the flexibility caused by the nearby hotspot mutation(s).

PANDA also regains transcriptional activities on most of the p53 target genes as shown in the heatmap of RNA expression level of a set of 127 p53 targets. RNA sequencing (RNA-seq) data also shows that among the reported 116 genes p53-activated targets, the majority of the target genes were up-regulated by PANDA-R282W, including the well-known p53 targets BBC3, BAX, TP5313, CDKN1A, and MDM2.

We solved the 3D structure of at least one mp53 at a resolution of approximately 1-3 Å (see FIG. 11 shows a 3D structure of the mp53, p53-R249S), identified a druggable pocket on p53 for the restoration of wildtype structure and function (“PANDA Pocket”) (see FIG. 1 showing the PANDA Pocket is located at the dorsal end of p53), and discovered that the PANDA Pocket is key to p53 structural stability. Importantly, the druggable PANDA Pocket can be used to screen p53 rescue compounds. We further discovered immobilizing the PANDA Pocket with a PANDA Agent would stabilize the mp53 structure. We further discovered that group of key residues played significant role in controlling the stability of PANDA Pocket (FIG. 14). These amino acid residues include S116, F134, Q136, T140, P142, and F270. For example, we found S116N, S116F and Q136R mutations on p53-G245S can rescue PIG3 transcriptional activity. Similarly, S116N and Q136R mutations on p53-G245S can rescue PUMA transcriptional activity. Based on our crystal structure (for example, of p53-R249S; p53-R249S with As; p53-G245S; and p53-G245S with As) and our mass spectroscopy results, we confirmed a single arsenic (or analogue) atom covalently binds the three cysteines C124, C135, and C141 (each a “PANDA Cysteine” and together a “PANDA Triad”) within the PANDA Pocket.

In certain embodiments, the PANDA Core is produced by a reaction between the PANDA Pocket and the PANDA Agent. Preferably, the reaction is mediated by an As, Sb, and/or Bi group oxidizing one or more thiol groups of PANDA Cysteines (PANDA Cysteines lose between one to three hydrogens) and the As, Sb, and/or Bi group of PANDA Agent is reduced (PANDA Agent loses oxygen). In certain embodiments, the PANDA Agent is the reduzate formed from having tightly associated with p53. In certain embodiments, the PANDA Agent is an arsenic atom, an antimony atom, a bismuth atom, any analogue thereof, combinations thereof, and the like.

In certain embodiments, the PANDA Agent transforms cancer-promoting mp53 to tumor suppressive PANDA and have significant advantages over existing therapeutic strategies such as by reintroducing wtp53 or promoting degradation/inactivation of endogenous mp53 in the patient. The PANDA Agent mediated mp53 rescue through PANDA, high rescue efficiency and mp53 selectivity are the two superior characteristics over previously-reported compounds. In certain embodiments, the PANDA Agent ATO can provide a near complete rescue of p53-R175H, from a level equivalent to about 1% of that of wtp53 to about 97% of that of wtp53 using the robust PAb1620 (also for PAb246) IP assay. In certain embodiments, the PANDA Agent ATO also provides a near complete rescue of the transcriptional activity of p53-G245S and p53-R282W on some pro-apoptotic targets, from a level equivalent to about 4% of that of wtp53 to about 80% of that of wtp53, using a standard luciferase reporter assay. In other embodiments, the PANDA Agent ATO can rescue the function of mp53s to a level that exceeds that of the wtp53, as shown, for example, in our luciferase assay for p53-1254T and p53-V272M. We have robustly reproduced these superior results, as compared to existing compounds, in numerous contexts and know no existing compound that can rescue the structure or transcriptional activity of a hotspot mp53 by a level equivalent to about 5% of that of wtp53 in our assays.

In certain embodiments, the PANDA Agent ATO and PANDA can selectively target Structural mp53 with strikingly high efficiency. In addition, Contracting mp53s can also be rescued with moderate efficiency. For example, we found a wide range of Structural mpS3s, including a large percentage of hotspot mp53s, can be efficiently rescued by the PANDA Agent ATO through the formation of PANDA. In addition, we also found that the Contacting mp53s can be rescued by ATO through PANDA with a limited efficiency. This remarkable property is not only superior but is conceptually different from most of the reported compounds, including CP-31398 (Foster et al., 1999), PRIMA-1 (Bykov et al., 2002), SCH529074 (Demma et al., 2010), Zinc (Puca et al., 2011), stictic acid (Wassman et al., 2013), p53R3 (Weinmann et al., 2008), and others that are reported to be able to rescue both types of mp53.

### 1.5 PANDA Agents, Compounds for Rescuing Mp53

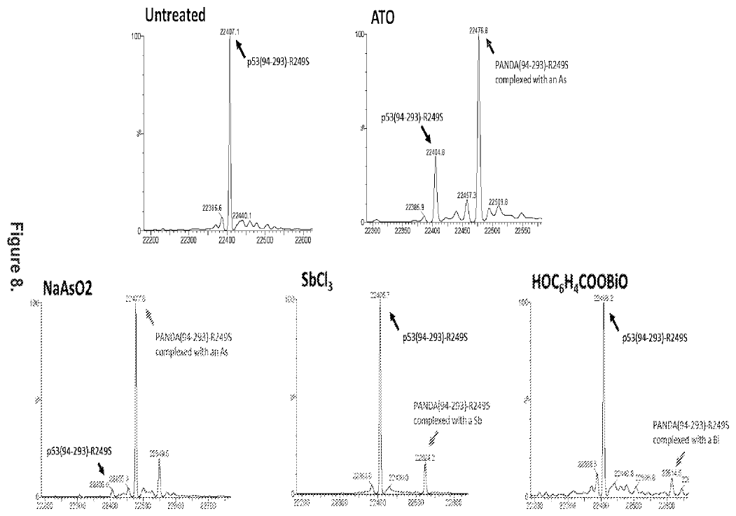

As used in this application, “PANDA” refers to the p53 and arsenic analogue complex. “PANDA Cysteine” refers to one of C124, C135, or C141. “PANDA Triad” refers to the C124, C135, C141 together. “PANDA Pocket” refers to the three-dimensional structure centered around PANDA Triad. The PANDA Pocket includes PANDA Triad and directly contacting residues (S116 contacts C124, C275 and R273 contact C135, Y234 contacts C141), residues adjacent to PANDA Triad (V122, T123, T125, and Y126; M133, F134, Q136, and L137; K139, T140, P142, and V143), and residues in 7-A distance to PANDA Triad (L114, H115, G117, T118, A119, K120, S121, A138, 1232, H233, N235, Y236, M237, C238, N239, F270, E271, V272, V274, A276, C277, P278, G279, R280, D281, and R282) (FIG. 13). “PANDA Core” refers to the PANDA Pocket with a PANDA Agent bounded to it. “PANDA Agent” refers to the rescue agent capable of forming at least one tight association with the PANDA Pocket. PANDA Agent can be any compound that efficiently stabilizes mp53 by binding potentials to the PANDA Pocket. Preferably, the PANDA Agent enhances Tₘ of mp53 by about 3-100 times of those of PRIMA-1, and/or folds mp53 by about 3-100 times of those of PRIMA-1, and/or stimulates mp53's transcriptional activity by about 3-100 times of those of PRIMA-1. Preferably, PANDA Agent has at least one cysteine binding potentials, further preferably two or more cysteine binding potential, and further preferably three or more cysteine binding potential. Further preferably, PANDA Agent is compound containing one or more As, Bi or Sb atom. Further preferably, PANDA Agent can be selected from the thousands of compounds listed in Table 1-Table 6, which we have predicted to efficiently bind PANDA Cysteines and efficiently rescue mp53 in situ. More preferably, PANDA Agent is one of the 33 compounds listed in Table 7, which we had experimentally confirmed to rescue mp53's structure and transcriptional activity. More preferably, PANDA Agent include the arsenic analogues such as As₂O₃, NaAs₂, SbCl₃, and HOC₆H₄COOBiO which we confirmed to directly bind p53-R249S (FIG. 8); and As₂O₃, HOC₆H4COOBiO, Bil₃, SbI₃, and C₆H₄K₂O₁₂Sb₂.xH2O. which we have shown to stabilize mp53 structure (see discussions in Section 1.5).

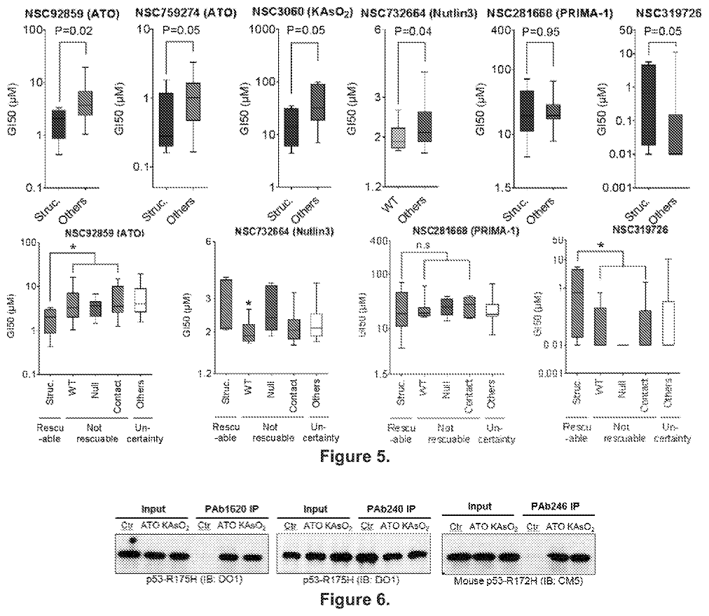

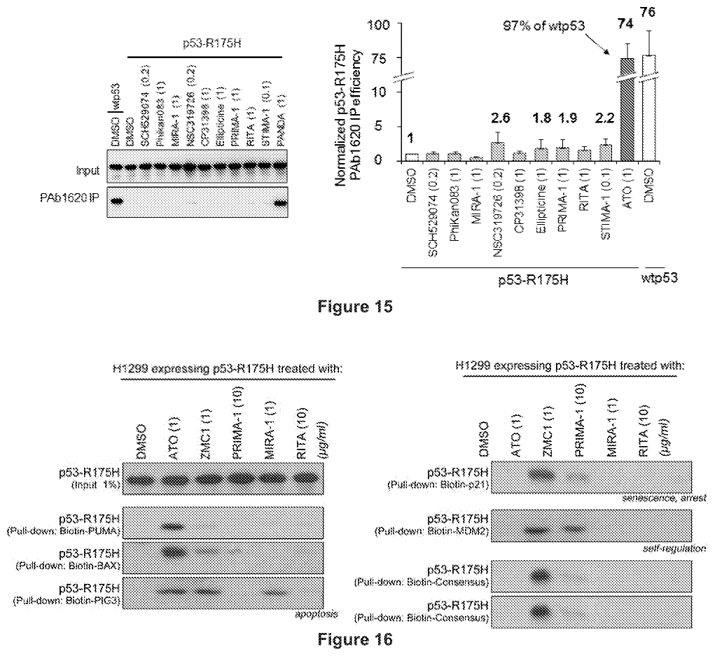

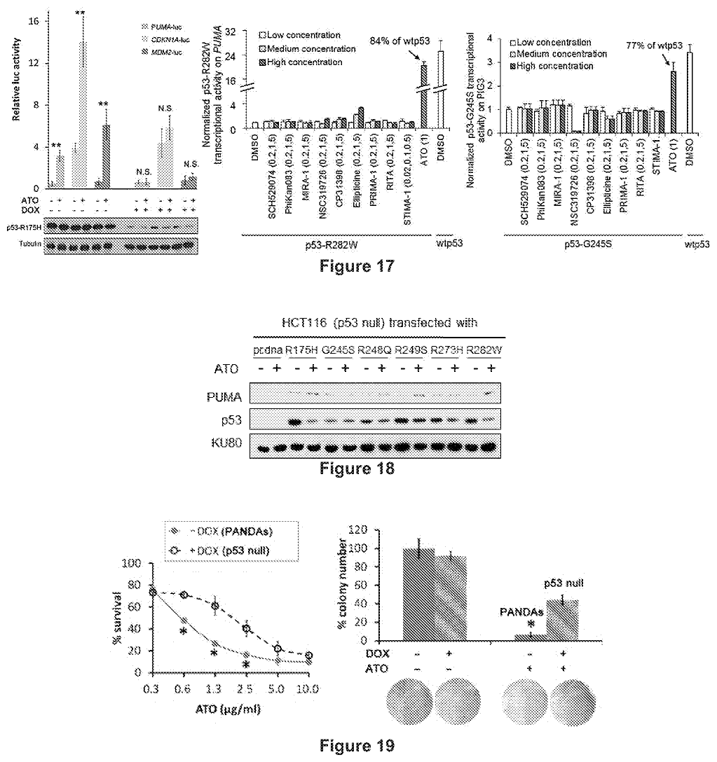

We discovered that in general, compounds with one or more cysteine-binding potentials on p53, preferably two or more cysteine-binding potential on p53, and more preferably three cysteine-binding potential on p53 are good rescue compounds for a broad spectrum of mp53s. Some of these compounds can rescue mp53 to near wildtype-like conditions (see FIG. 15 and FIG. 17). For example, we showed that of the 47 arsenic-containing compounds in the DTP library, those with one or more cysteine binding potentials have significantly similar NCI60 inhibition profiles as the ATO, an mp53 rescue agent with strong structural and functional rescue capacity (see Table 9, and FIG. 5-FIG. 10). Among these, compounds with three or more cysteine binding potential (e.g.: NSC3060 (KAsO₂, Pearson's correlation 0.837, p<0.01), NSC157382 (Pearson's correlation 0.812, p<0.01), and NSC48300 (4 cysteine-binding potential; Pearson's correlation of 0.627, p<0.01)) have higher similarity to ATO than compounds with two cysteine binding potential (NSC92909, Pearson's correlation 0.797, p<0.01; NSC92915, Pearson's correlation 0.670, p<0.01; NSC33423, Pearson's correlation 0.717, p<0.01), which in turn has higher similarity than compounds with one cysteine binding potential, (NSC727224, Pearson's correlation 0.598, p<0.01; NSC724597, Pearson's correlation 0.38, p<0.01; NSC724599, Pearson's correlation 0.553). We further found that As, Sb, and/or Bi compounds with mono-cysteine binding potential (e.g.: NSC721951), bi-cysteine binding potential (e.g.: NSC92909), or tri-cysteine binding potential (e.g.: NAS3060) can rescue mp53's structure and transcriptional activity (Table 7). Moreover, compounds that has three or more cysteine binding potential having the highest rescue efficiency, followed by compounds with bi-cysteine binding potential, and followed by compounds with mono-cysteine binding potential (see Table 7; see also equations (1)-(6)).

We further suggest other non-As, Sb, and Bi compounds can also serve as efficient a PANDA Agent as long as they can bind PANDA pocket which leads to mp53 stability. These compounds can contain group of thiols (e.g.: 1,4-Benzenedithiol), Michael acceptor (e.g.: (1E,6E)-1,7-Diphenyihepta-1,6-diene-3,5-dione), and others which can bind cysteine. These compounds can also lack of cysteine-binding ability, however, they bind other residues of PANDA pocket to stabilize mp53.

We further discovered that the preferred rescue compounds for mp53 can (i) upon hydroxylation, simultaneously bind to one or more mp53 cysteines, preferably two or more mp53 cysteines, more preferably three mp53 cysteines; (ii) can form at least one tight bond to PANDA Pocket; (iii) can increase the ratio of folded p53 to unfolded p53 and/or refold mp53 with high efficiency, at levels comparable to that of wtp53 in some cases (as measured by immunoprecipitation with, for example, PAb1620 and/or PAb246); (iv) can rescue the transcriptional activity of mp53s at levels comparable to that of wtp53 in some cases (as measured by, for example, luciferase report assay); (v) can stabilize p53 and increase the melting temperature of mp53; (vi) can selectively inhibit mp53 expressing cell lines, such as the NCI60 cell lines that expresses the Structural hotspot mp53; (vii) can inhibit mouse xenografts dependent on Structural mp53s; and/or (viii) can be used to treat mp53 harboring cancer patients in combination with DNA-damaging agents.

We further discovered that elemental arsenic, elemental bismuth, elemental antimony, and compounds containing elemental arsenic, bismuth, and/or antimony are good rescue compounds for mp53. We showed that arsenic, bismuth, and antimony containing compounds can stabilize the structure of mp53s and/or rescue its transcriptional activities (see Table 7). The arsenic-, bismuth-, and antimony-mediated mp53 rescue is achieved by binding of the released arsenic, bismuth, and antimony to mp53. For example, mass spectroscopy data showed arsenic, bismuth, and antimony atom binds to mp53 directly and covalently (see FIG. 8 showing single atom molecular weight increase under denaturing conditions) at 1:1 atom:mp53 ratio (or 0.93±0.19 arsenic per p53, as measured by inductively coupled plasma mass spectroscopy (“ICP-MS”)). The arsenic-, bismuth-, and antimony-mediated mp53 rescue also elevates mp53 Tₘ. For example, mp53 Tₘ increased by 1° C.-8° C. for As₂O₃, 1.85° C. for HOC₆H₄COOBiO, 0.86° C. for BiI₃, 3.92° C. for SbI₃, 2.95° C. for C₈H₄K₂O₁₂Sb₂.H2O. Moreover, these rescue compounds can also rescue one or more mp53s. For example, As₂03, HOC₆H4000BiO, BiI₃, SbI₃, C₈H₄K₂O₁₂Sb₂.H2O can rescue at least p53-R175H, p53-V272M, and p53-R282W, and are expected to also rescue the rescuable mp53s in Table 9.

We further discovered that the following six classes of compounds are preferred mp53 rescue compounds: a three-valence arsenic containing compound, preferably the compound can be hydrolyzed, further preferably the compound does not have a carbon-arsenic bond, further preferably the compound is one that is listed in Table 1; a five-valence arsenic containing compounds, preferably the compound can be hydrolyzed, further preferably the compound does not have a carbon-arsenic bond, further preferably the compound is one that is listed in Table 2); a three-valence bismuth containing compounds, preferably the compound can be hydrolyzed, further preferably the compound does not have a carbon-bismuth bond, further preferably the compound is one that is listed in Table 3; a five-valence bismuth containing compounds, preferably the compound can be hydrolyzed, further preferably the compound does not have a carbon-bismuth bond, further preferably the compound is one that is listed in Table 4; a three-valence antimony containing compounds, preferably the compound can be hydrolyzed, further preferably the compound does not have a carbon-antimony bond, further preferably the compound is one that is listed in Table 5; and five-valence antimony containing compounds, preferably the compound can be hydrolyzed, further preferably the compound does not have a carbon-antimony bond, further preferably the compound is one that is listed in Table 6. We arrived at the lists of compounds in Table 1-Table 6 by analyzing, in silico, approximately 94.2 million compounds derived from PubChem (https://pubchem.ncbi.nlm.nih.gov/), using the selection criteria of (i) compounds containing elemental arsenic or its analogues, such as antimony, and bismuth and (ii) the capacity to simultaneously bind to 3 cysteines (our compounds listed in Table 1-Table 6 are predicted to rescue mp53 with very high efficiency because they can simultaneously bind 3 cysteines of PANDA triad). These rescue compounds include three-valence and five-valence arsenic, three-valence and five-valence antimony, and three-valence and five-valence bismuth. The discovery of compounds containing Bi and/or Sb, and As, Sb, and/or Bi compounds with mp53 rescue capacity has tremendous clinical value because these compounds generally have lower toxicities than inorganic As compounds in the body.

Exemplary embodiments of the rescue compound can include any one of the Formulas I-XV.

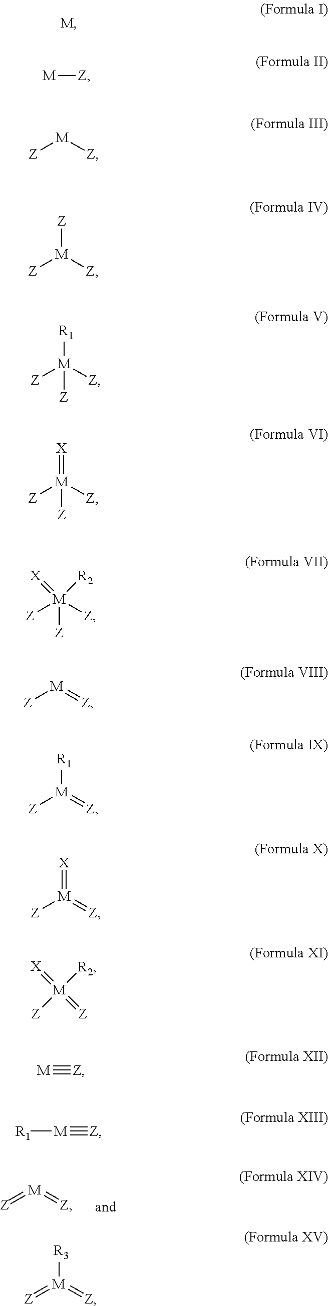

wherein:
M is an atom selected from a group consisting of As, Sb, and Bi;
Z is a functional group comprising a non-Carbon atom that forms a bond with M,
   wherein the non-Carbon atom is preferably selected from the group consisting of H, D, F, Cl, Br, I, O, S, Se, Te, Li, Na, K, Cs, Mg, Cu, Zn, Ba, Ta, W, Ag, Cd, Sn, X, B, N, P, Al, Ga, In, TI, Ni, Si, Ge, Cr, Mn, Fe, Co, Pb, Y, La, Zr, Nb, Pr, Nd, Sm, Eu, Gd, Dy, Tb, Ho, Er, Tm, Yb, and Lu;

wherein:
R₁ is selected from 1 to 9 X groups;
R₂ is selected from 1 to 7 X groups;
R₃ is selected from 1 to 8 X groups; and
wherein each X group comprises an atom that forms a bond with M; and

wherein:
each of M, the non-Carbon atom, and the atom has the appropriate charge, including no charge, in the compound;
each of Z and X is independently selected and can be the same or different from the other Z or X in the compound, respectively; and
each of the M, non-Carbon atom and the atom can be a part of a ring member.

In the preferred embodiment, the non-Carbon atom is selected from the group consisting of O, S, N, X, F, Cl, Br, I, and H.

Exemplary rescue compound with the structure of Formula I includes

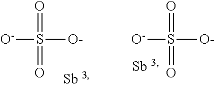 [SMILES: O=S(=O)([O-])[O-] ; O=S(=O)([O-])[O-]]

### As\hat{ }\hat{ }\hat{ } (CID No. 5,359,596), and

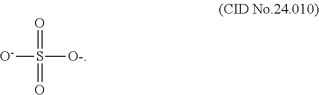

Exemplary rescue compound with the structure of Formula II includes

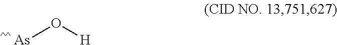

Exemplary rescue compound with the structure of Formula III includes As⁺(OH)₂.

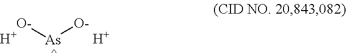

Exemplary rescue compound with the structure of Formula V includes

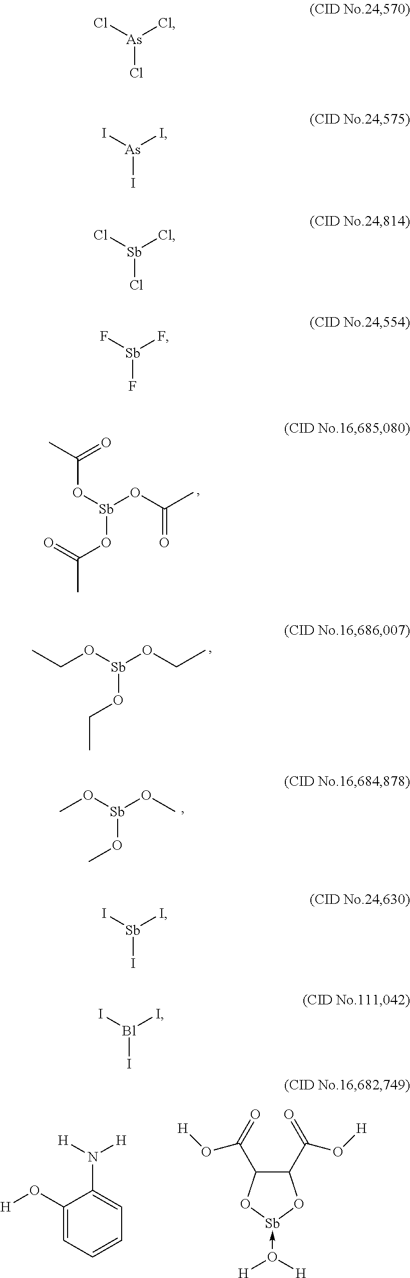

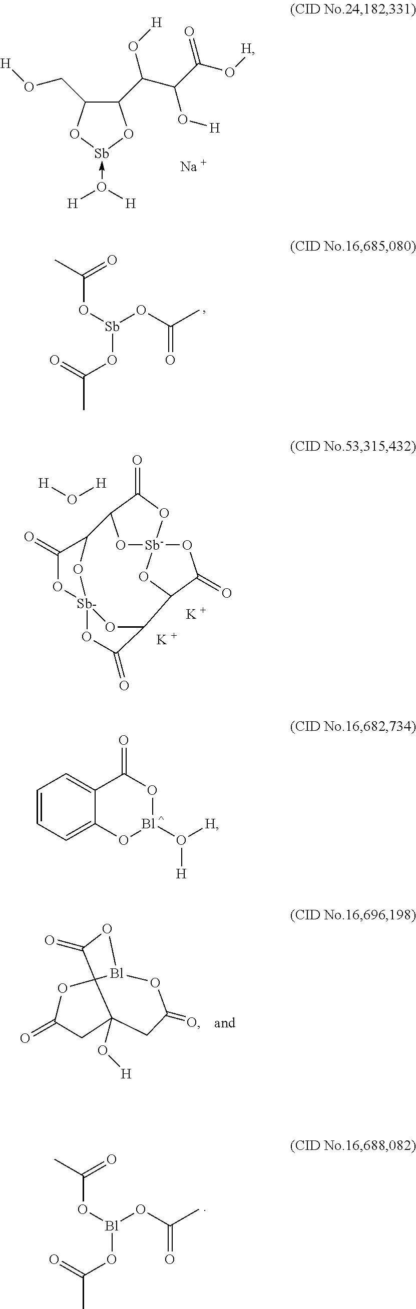

Exemplary rescue compound with the structure of Formula V includes

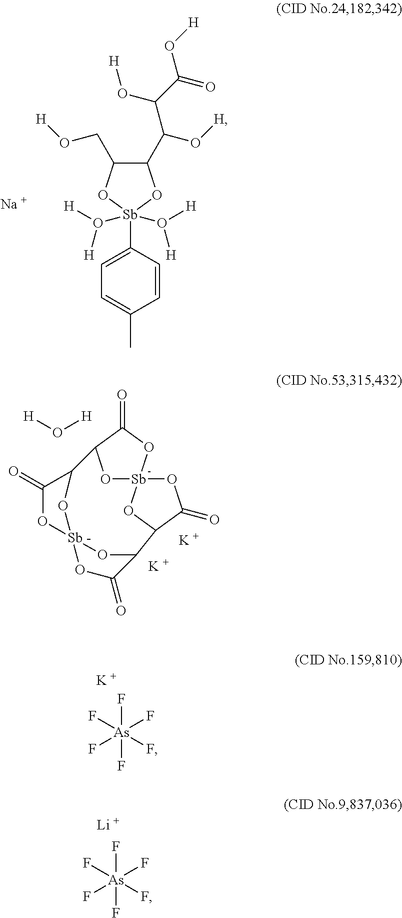

and.

Exemplary rescue compound with the structure of Formula VI includes

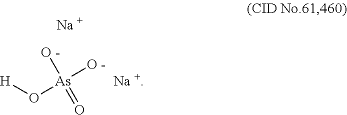

Exemplary rescue compound with the structure of Formula VIII includes

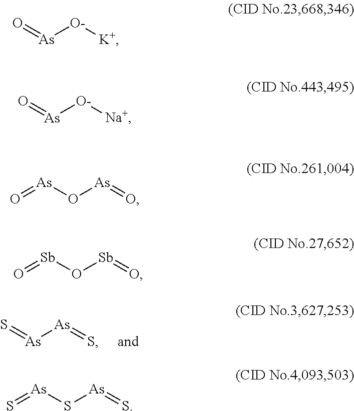

Exemplary rescue compound with the structure of Formula IX includes.

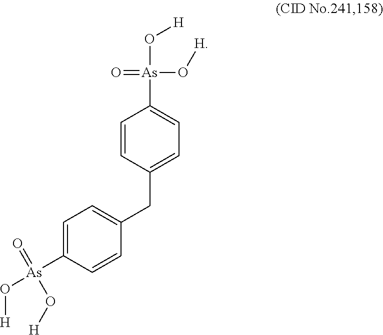

Exemplary rescue compound with the structure of Formula X includes

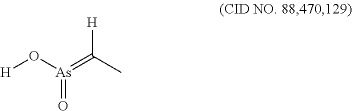

Exemplary rescue compound with the structure of Formula XII includes

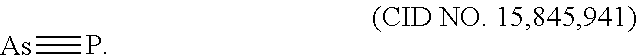

Exemplary rescue compound with the structure of Formula XIII includes

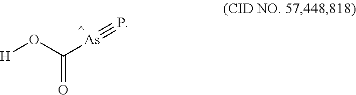

Exemplary rescue compound with the structure of Formula XV includes

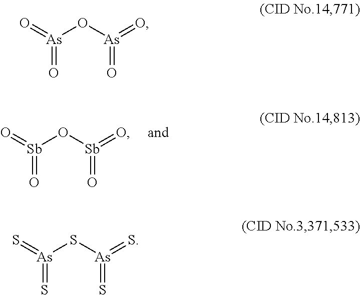

The following Equation (1) is an reaction for PANDA Agent. A compound containing M group with a Z₁ (a first group with the capacity to bind a first cysteine) and/or a Z₂ (a second group with the capacity to bind a second cysteine) and/or a Z₃ (a third group with the capacity to bind a third cysteine), Examples of Z₁, Z₂, and Z₃ includes O, S, N, X, F, Cl, Br, I, OH, and H. Z₁, Z₂, and/or Z₃ can bind to each other. M group includes for example a metal, such as an bismuth, a metalloid, such as an arsenic and an antimony, a group such as a Michael acceptor and/or a thiol, and/or any analogue with cysteine-binding ability. The PANDA Agent can undergo a hydrolysis before reacting and binding to p53 forming PANDA. In some cases, when a group cannot undergo hydrolysis, and accordingly cannot bind to a cysteine. In such cases, the remaining group(s) with cysteine binding potential binds to p53. X₁ and X₂ represent any groups bound to M. X₁ and/or X₂ can also be empty. X₁ and/or X₂ can also be able to bind cysteine.

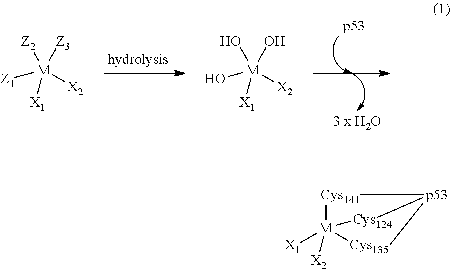

The following Equations (2) and (3) is an exemplary reaction for a PANDA Agent with tri-cysteine binding potential. 3-valence ATO or KAsO₂ undergoes hydrolysis, covalently binds to three PANDA Cysteines on p53.

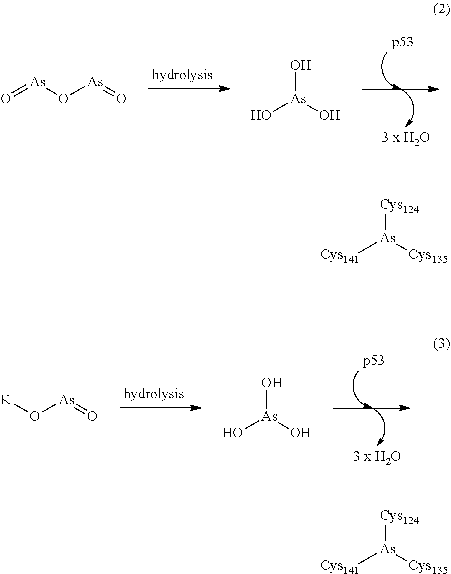

The following equation (4) is an exemplary reaction for a PANDA Agent with tri-cysteine binding potential. 5-valence As compound undergoes hydrolysis, covalently binds to three PANDA Cysteines on p53.

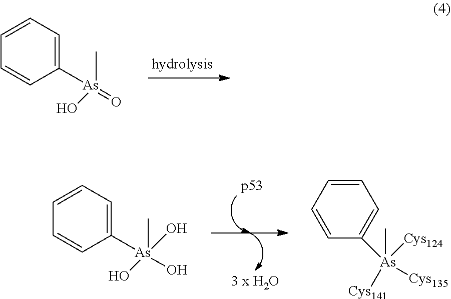

The following equation (5) is an exemplary reaction for a PANDA Agent with bi-cysteine binding potential. The PANDA Agent can bind to PANDA Cysteines, or to PANDA Cysteines (Cys₁₂₄, Cys₁₃₅, or Cys₁₄₁), or Cys₂₇₅ and Cys₂₇₇ or C₂₃₈ and C₂₄₂.

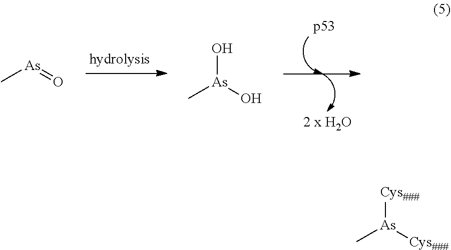

The following equation (6) is an exemplary reaction for a PANDA Agent with mono-cysteine binding potential. The PANDA Agent can bind to PANDA Cysteines, (i.e. Cys₁₂₄, Cys₁₃₅, or Cys₁₄₁) or the other 3 cysteines on PANDA Pocket (Cys₂₃₆, Cys₂₇₅, or Cys₂₇₇).

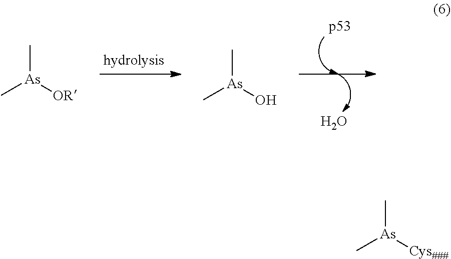

We further discover that KAsO₂, AsCl₃, HAsNa₂O₄, NaAsO₂, AsI₃, As₂O₃, As₂O₅, KAsF₆, LiAsF₆, SbCl₃, SbF₃, SbAc₃, Sb₂O₃, Sb(OC₂H₅)₃, Sb(OCH₃)₃, SbI₃, Sb₂O₅, Sb₂(SO₄)₃, BiI₃, C₁H₁As₂N₄O₂, C₁₃H₁₄As₂O₆, C₁₇H₂₈AsClN₄O₆S, C₁OH₁₃NO₆Sb, C₆H₁₂NaO₆Sb+, (CH₃CO₂)₃Sb, C₆H₄K₂O₁₂Sb₂.xH₂O, C₁₃H₂₁NaO₉Sb+, HOC₆H₄COOBiO, [O₂CCH₂C(OH)(CO₂)CH₂CO₂]Bi, (CH₃CO₂)₃Bi, As₂S2, As₂S3, and As₂S₅ are remarkable mp53 rescue compounds, capable of rescuing both the structure and transcriptional function of mp53 in experimental assays (see Table 7). For example, we tested some structural mp53s for their abilities to refold protein, increase Tₘ, and stimulate transcriptional activity. Among these preferred mp53 rescue compounds, We discovered that As₂O₃ was previously approved by the U.S. Food and Drug Administration to treat acute promyelocytic leukemia (“APL”) in 2000 as NDA 21-248, but was not approved to treat other cancer types yet, because it did not provide any statistically significant efficacy. Additionally, the PANDA Agent Fowler's solution (KAsO₂) has significant side-effects and are not used in clinical settings any more in past decades, but this may now be overcome by selecting and treating a patient with rescuable mp53, as disclosed in this Application. The PANDA Agent As₄S4 has been shown to be as effective as conventional intravenous ATO in treating APL patients, but unlike ATO, As₄S4 can be conveniently orally administrated (Zhu et al., 2013), making particularly attractive cancer therapy. Furthermore, we also discover that PANDA Agents As₂S3, As₂S2, and As₂S5, which have strong ability to rescue mp53, can also be formulated for oral administration.

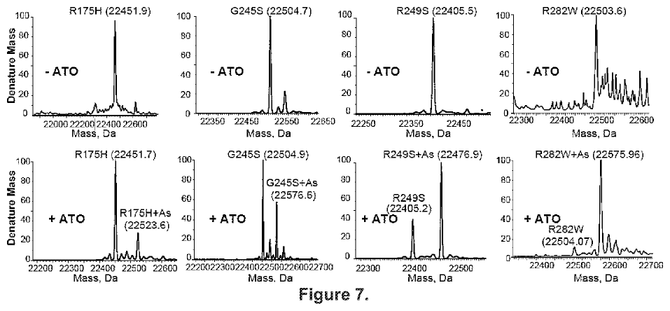

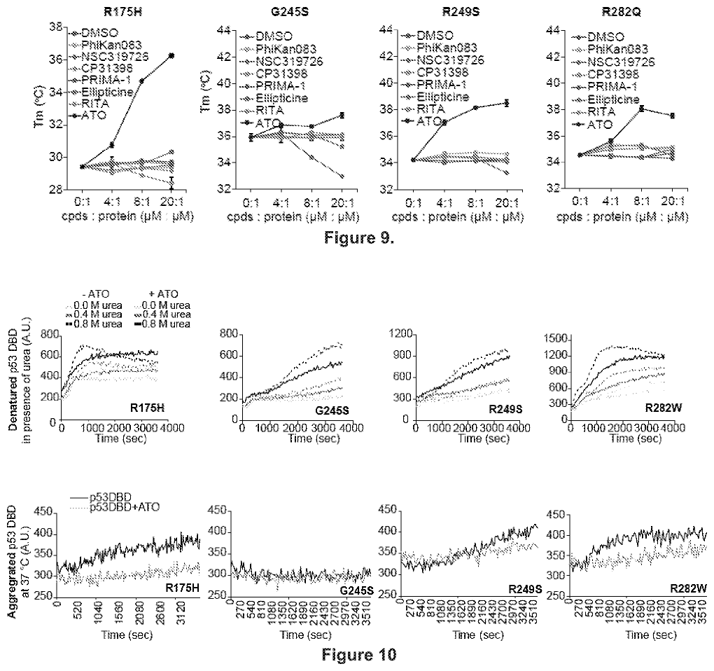

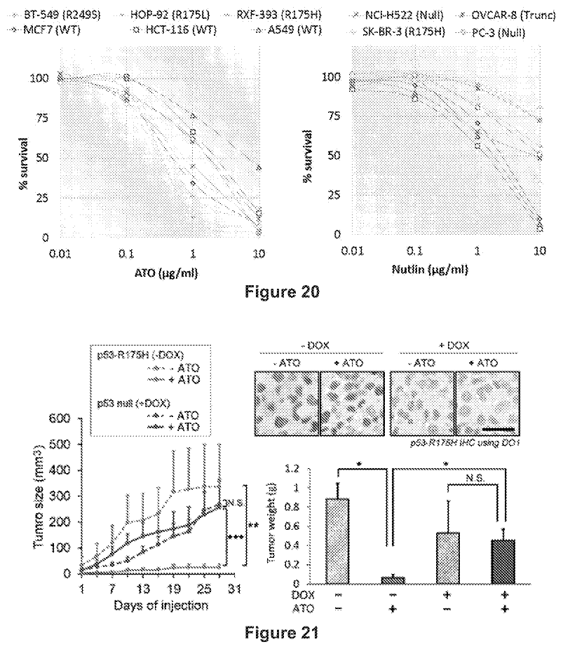

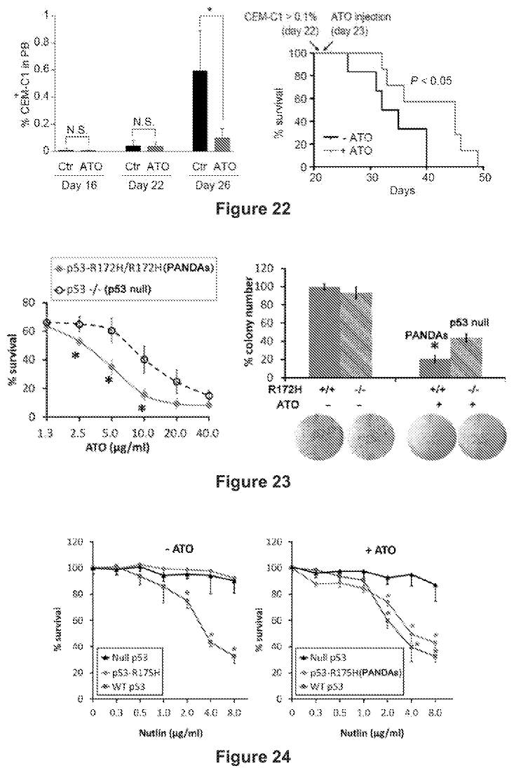

We further discovered that arsenic trioxide (ATO: NSC92859 & NSC759274) and potassium arsenite (KAsO₂: NSC3060) are two wide-spectrum mp53 rescuing agents with remarkably high rescue efficiency (Table 7, Table 9 and FIG. 12). For example, As₂O₃ increased wtp53-like structures of p53-R175H by approximately 50-100 fold to a level equivalent to about 97% of wtp53 (see FIG. 15); increased wtp53-like transcriptional activity of p53-R282W by approximately 21-fold, to a level equivalent to about 84% of wtp53 (FIG. 12 and FIG. 17); and increased wtp53-like transcriptional activity of p53-G245S by approximately 3-fold, to a level equivalent to about 77% of wtp53 (FIG. 12 and FIG. 17). We demonstrated that both ATO and KAsO₂ can, among others, (i) rescue mp53 structure (see FIG. 6 showing a measurable increase of folded PAb1620 human epitope and PAb246 mouse epitope and a measurable decrease of the PAb240 epitope; see also Table 7); (ii) rescue mp53's DNA binding ability (see FIG. 16, showing ATO rescued p53-R175H DNA binding ability with respect to MDM2, which is involved in p53 self-regulation; CDKN1A, which encoding p21 protein and is involved in senescence, invasion, metastasis, cell stemness and cell cycle arrest; PIG3, which is involved in apoptosis; PUMA, which is involved in apoptosis; BAX, which is involved in apoptosis; and the p53-binding consensus sequence); (iii) rescue mp53's transcriptional activity (see FIG. 5, FIG. 12, and FIG. 17; see also Table 7); (iv) increase the production of p53 downstream mRNA such as MDM2, PIG3, PUMA, CDKN1A, and BAX, in about 24 hr; (v) increase production of downstream p53 protein, such as PUMA, BAX, PIG3, p21, and MDM2 in about 48 hours (see FIG. 18); (vi) rescue mp53's tumor suppressive function in vitro (see FIG. 5), in human cells (see FIG. 19), in mouse cells (see FIG. 23); (vii) rescue mp53's tumor suppressive function in vivo, including in solid tumor xenograft model (see FIG. 21) and hematological malignance xenograft model (FIG. 22); (viii) inhibit malignancies (see FIG. 20); (ix) rescue different mp53s (see FIG. 5, Table 7, Table 9 and FIG. 12); (x) and has remarkable rescue capacity for Structural mp53s (FIG. 5). These experimental data are further supported by our atom-level rescue mechanism, which includes hydrolyzing the rescue agent (see equations (1)-(6)) and binding to p53 (see equations (1)-(6)) and FIG. 7 showing mass spectroscopy data supporting direct and covalent association), thereby increasing the stability of mp53 folded state (see FIG. 9 showing an increase of mp53 Tₘ by approximately 1° C.-8° C.), and inhibiting the denatured and aggregated state of mp53 (as shown, for example, in non-denaturing PAGE and western blot; see also FIG. 10). Compared to PRIMA-1 and its analogue PRIMA-1MET, which is under phase II clinical trial (Bauer et al., 2016; Joerger and Fersht, 2016), and which increasingly have been suggested to target oxidative stress signaling components, our PANDA Agents are highly effective and specific towards a diverse number of mp53, with low off targeting (see FIG. 28; see also Table 9).

The PANDA Agent comprising a three and/or five valence arsenic is generally effective in treating cancer in a subject, including an animal, at a dose at a wide range of dosages by intravenous injection and oral administration. In certain embodiments, the daily dosage is from about 0.5 mg/kg to about 50 mg/kg, preferably from about 0.5 mg/kg to about 25 mg/kg, more preferably from about 1 mg/kg to about 25 mg/kg, more preferably from about 1 mg/kg to about 15 mg/kg, more preferably from about 1.7 mg/kg to about 15 mg/kg, and more preferably from about 1.7 mg/kg to about 5 mg/kg. In certain embodiments, the dose is about 5 mg/kg. In certain embodiments, the PANDA Agent ATO is administered by intravenous injection or by oral administration at 1 mg/ml concentration, at a dose of 5 mg/kg per day.

In other embodiments, the daily dosage is from about 10 mg/kg to about 1000 mg/kg, preferably from about 10 mg/kg to about 500 mg/kg, more preferably from about 20 mg/kg to about 500 mg/kg, more preferably from about 20 mg/kg to about 300 mg/kg, more preferably from about 33 mg/kg to about 300 mg/kg, and more preferably from about 33 mg/kg to about 100 mg/kg. In certain embodiments, the dose is about 100 mg/kg. In certain embodiments, the PANDA Agent As₂S2, As₂S3, As₂S5, and As₄S4 is administered by oral administration at 15 mg/L concentration, at a dose of 100 mg/kg

The PANDA Agent comprising a three valence and/or five valence antimony is generally effective in treating cancer in a subject, including an animal, at a dose at a wide range of dosages by intravenous injection and oral administration. In certain embodiments, dosage is from about 60 mg/kg to about 6000 mg/kg, preferably from about 60 mg/kg to about 3000 mg/kg, more preferably from about 120 mg/kg to about 3000 mg/kg, more preferably from about 120 mg/kg to about 1500 mg/kg, more preferably from about 150 mg/kg to about 1200 mg/kg, and more preferably from about 300 mg/kg to about 1200 mg/kg. In certain embodiments, the dose is about 600 mg/kg. In certain embodiments, the PANDA Agent is administered by intravenous or oral administration at 100 mg/ml concentration, at a dose of 600 mg/kg per day.

The PANDA Agent comprising a three valence and/or five valence bismuth is generally effective in treating cancer in a subject, including an animal, at a dose at a wide range of dosages by intravenous injection and oral administration. In certain embodiments, the daily dosage is from about 60 mg/kg to about 6000 mg/kg, preferably from about 60 mg/kg to about 3000 mg/kg, more preferably from about 120 mg/kg to about 3000 mg/kg, more preferably from about 120 mg/kg to about 1500 mg/kg, more preferably from about 150 mg/kg to about 1200 mg/kg, and more preferably from about 300 mg/kg to about 1200 mg/kg. In certain embodiments, the dose is about 600 mg/kg. In certain embodiments, the PANDA Agent is administered by intravenous or oral administration at 100 mg/ml concentration, at a dose of 600 mg/kg per day.

The PANDA Agent comprising a three and/or five valence arsenic is generally effective in treating cancer in a human at a wide range of dosages by intravenous injection and oral administration. In certain embodiments, the effective dose results in a maximum As concentration in the patient's blood (plasma) from about 0.094 mg/L to about 9.4 mg/L, preferably from about 0.094 mg/L to about 4.7 mg/L, more preferably from about 0.19 mg/L to about 4.7 mg/L, more preferably from about 0.31 mg/L to about 2.82 mg/L, more preferably from about 0.31 mg/L to about 1.31 mg/L, more preferably from about 0.57 to about 1.31 mg/L. In certain embodiments, the daily dose is from about 0.67 mg/kg to about 12 mg/kg, more preferably from about 0.2 to about 4.05 mg/kg, wherein the maximum As concentration is about 0.57 mg/L to about 1.31 mg/L, and wherein the platform As concentration in blood (plasma) is from about 0.03 mg/L to about 0.07 mg/L. In certain embodiments, the PANDA Agent is ATO, As₂S₂, As₂S₃, As₂S₅, and As₄S₄.

The PANDA Agent comprising a three and/or five valence antimony is generally effective in treating cancer in a human at a wide range of dosages by intravenous injection and oral administration. In certain embodiments, the effective dose results in a maximum Sb concentration in the patient's blood (plasma) from about 3.58 mg/L to about 357.5 mg/L, preferably from about 3.58 mg/L to about 179 mg/L, more preferably from about 7.15 mg/L to about 179 mg/L, more preferably from about 7.15 mg/L to about 107 mg/L, more preferably from about 12 mg/L to about 107 mg/L, more preferably from about 32.7 to about 38.8 mg/L. In certain embodiments, the daily dose is from about 20 mg/kg, wherein the maximum Sb concentration is from about 32.7 mg/L to about 38.8 mg/L, and wherein the platform Sb concentration in blood (plasma) is about 3.5 mg/L.

The PANDA Agent comprising a three and/or five valence bismuth is generally effective in treating cancer in a human at a wide range of dosages by intravenous injection and oral administration. In certain embodiments, the effective dose results in a maximum Bi concentration in the patient's blood (plasma) from about 3 mg/L to about 300 mg/L, preferably from about 3 mg/L to about 150 mg/L, more preferably from about 6 mg/L to about 150 mg/L, more preferably from about 6 mg/L to about 90 mg/L, more preferably from about 10 mg/L to about 90 mg/L, more preferably from about 30 mg/mL. In certain embodiments, the daily dose is from about 20 mg/kg, wherein the maximum Bi concentration is from about 32.7 mg/L to about 38.8 mg/L, and wherein the platform Bi concentration in blood (plasma) is about 3.5 mg/L.

We further discovered that combining ATO and other approved drugs can be effective to treating cancer. For example, we found the combination therapy of ATO and a DNA-damaging agents can treat patients with AML and MDS. Results from our phase I Decitabine (“DAC”)—ATO combination therapy trial for Myelodysplastic Syndrome (DMS) showed complete remission for the two patients that harbored rescuable mp53s (Table 11 and FIG. 26). DAC is a cytidine analog and first-line drug for MDS patients that binds to, causes damages to, and demethylates DNA. In this ongoing trial, which was approved by hospital ethics committee, we recruited 50 MDS patients, sequenced their TP53 exomes, and found patients #27, #35, and #49 harbored p53 mutations (mp53 variant allele fraction >10%) (Table 11 and FIG. 26). Among them, patients #27 and #35 harbored ATO rescuable p53-S241F and p53-S241C respectively, and are selected to be treated under the trial, while patient #49 harbored non-rescuable p53-R273L, and was not selected for trial treatment (FIG. 26; see also Table 8 and Table 9). Under the trial conditions, patients #27 and #35 were administered a treatment cycle of 25 mg of DAC and 0.2 mg/kg of ATO by intravenous guttae (“ivgtt”) every four weeks. For each cycle, DAC was administered on days 1, 2 and 3 and ATO was administered on days 3, 4, 5, 6, and 7. Patients #27 and #35 were monitored throughout the treatment and their minimal residual disease (“MRD”), bone marrow blast cells (“BM blast”), white blood cell count (“WBC”), haemagglutinin count (“Hb”), and platelet count (“PLT”) were measured periodically (see FIG. 26). Cancer cells were eliminated (blast cells detected to be <5%, i.e. “complete remission”) for Patient #27 and #35 for about 8 and 7 months respectively (see FIG. 26). In the reported standard DAC mono-treatment, where 101 MDS patients were treated without mp53 selection, only 27 patients achieved complete remission for 4-48 month, while the remaining 74 patients did not achieve complete remission (complete remission duration 0 month) (Chang et al, 2016). Thus, patients benefited statistically significantly more from the DAC-ATO combination regimen judging by the complete remission duration (P=0.0406). In standard DAC mono-treatment for 14 MDS patients expressing mp53, only 9 patients achieved complete remission for 3-14 month (i.e.: 3, 3, 4, 4, 6, 6, 10, 12, and 14 months), while the remaining 5 patients did not achieve complete remission (complete remission duration 0 month). Thus, even patients with mp53s benefited more from the DAC-ATO combination treatment as compared to the DAC mono-treatment (P=0.0013).

We also identified patient #19, who harbored wtp53 during initial screening, but later developed DAC treatment related rescuable p53-Q038H and p53-Q375X on the 8th month of the DAC mono-treatment (see FIG. 26). At this time point, disease progression was fast, with the MDS expected to transform to AML in 1 month and patient #19 was expected to not survive beyond 2-4 months. Accordingly, patient #19 was administered a treatment cycle of 25 mg of DAC, 0.2 mg/kg of ATO, and 25 mg of ARA-c of ARA by intravenous guttae (“ivgtt”) every four weeks. For each cycle, DAC was administered on days 1, 2 and 3; ATO was administered on days 3, 4, 5, 6, and 7; and ARA is administered on days 1, 2, 3, 4, and 5. Like patients #27 and #35, patient #19 was also responsive to the combination therapy. The combination treatment with ATO and ara-C was effective in patient #19 even though the 8-month DAC mono-treatment still resulted in a fast progressed disease. In particular, upon the combination treatment cancer cells did not increases significantly for 6 month.

Taken together, we have discovered that ATO is effective in treating cancer patients, such as MDS patients, particularly those harboring mp53s rescuable mutation. We further discovered that the efficacy of treatment can be improved by (1) obtaining a sample from the patient and sequencing patient's p53, (2) determining whether the mp53 is rescuable or not, and (3) administering an effective amount of one or more PANDA Agent, such as ATO and/or other drug candidates alone or in combination with other effective cancer drugs to the patient; selecting patients with p53 mutations on residues most responsive to ATO, such as mutations on S241C and S241F. Importantly, we have determined that the ATO rescuable mp53 includes: R175H, R245S, R248Q, R249S, R282W, I232T, F270C, Y220H, I254T, C176F, H179R, Y220C, V143A, S033P, D057G, D061G, Y126C, L130H, K132M, A138V, G154S, R156P, A159V, A159P, Y163H, Y163C, R174L, C176Y, H179Y, C238Y, G245A, G245D, R248W, G266R, F270S, D281H, D281Y, R283H, F054Y, SO90P, Q375X, Q038H, R156P, A159V, A159P, Y163H, Y163C, R174L, C176Y, H179Y, H179Q, P190L, H193R, R209K, V216E, Y234H, M2371, V272M, S241A, S241C, S241D, S241E, S241F, S241G, S241H, S2411, S241L, S241M, S241N, S241P, S241Q, S241R, S241T, S241V, S241W, and S241Y (see Table 8, mp53s that are indicated as either structurally rescuable or functionally rescuable). Additionally, we have determined that the ATO non-rescuable mp53s includes: R273H, R273C, R278S, S006P, L014P, Q052R, P072A, P080S, T081P, S094P, S095F, R273S, R273L, P278H, L383P, M384T, S241K (see Table 8 mp53s that are indicated as neither structurally rescuable nor functionally rescuable).

mp53 is associated with considerably poor overall survival and prognosis of a wide range of cancers, including myeloid leukemia (AML/MDS) patients (Cancer Genome Atlas Research et al., 2013; Lindsley et al., 2017). Under NCCN guidelines, the majority of recommended AMLMDS treatments, aside from APL, are DNA-damaging agents. These DNA-damaging agents are known to activate wtp53 function to kill cancer cells through p53 post-translational modifications (“PTM”s) (Murray-Zmijewski et al., 2008). These PTMs include, for example, phosphorylation, acetylation, sumoylation, neddylation, methylation, and ubiquitylation.

Our discovery further shows that PANDA Agent ATO can be used for a wide range of ATO-responsive cancers in clinical trials. It is preferred that patient recruitment follow a specific, highly precise, recruitment prerequisite, in order to achieve maximum efficacy. While ATO was approved by FDA to treat acute promyelocytic leukemia (APL), a subtype of leukemia and intensively trialed, with the aim to broaden its application to non-APL cancer types over the past two decades, it has not yet been approved for this purpose. This is largely attributed to a failure to reveal an ATO-affecting cancer spectrum. Indeed, no mp53 dependency can be observed in the sensitivity profile of ATO on the NCI60 cell panel simply by differentiating lines into a mp53 group and a wtp53 group. Non-ATO rescue compounds were also extensively researched and some were identified, including, CP-31398; PRIMA-1; PRIMA-1-MET; SCH529074; Zinc; stictic acid, p53R3; methylene quinuclidinone; STIMA-1; 3-methylene-2-norbomanone; MIRA-1; MIRA-2; MIRA-3; NSC319725; NSC319726; SCH529074; PARP-PI3K; 5,50-(2,5-furandiyl)bis-2-thiophenemethanol; MPK-09; Zn-curc or curcumin-based Zn(II)-complex; P53R3; a (2-benzofuranyl)-quinazoline derivative; a nucleolipid derivative of 5-fluorouridine; a derivative of 2-aminoacetophenone hydrochloride; PK083; PK5174; and PK7088. However, they have low rescue efficacy.

The PANDA Agents we identified and described herein, including the PANDA Agents with Formulation I-XV, the PANDA Agents listed in Table 1-Table 6, and PANDA Agents listed in Table 7 show exceptional efficacy in rescuing mp53 with rescuable mutations (for example, those listed in Table 8) in vitro and in vivo, among others. Many of them have structures that are significantly different from ATO and have not previously been proposed for use in treating a p53 disorder. By separating rescuable mp53s from in a pool of patients with a p53 disorder, we have, for the first time, discovered a compound and method to effectively treat multiple types of p53 disorders, including multiple classes of cancers. The size of the class is considerably large, covering an estimated amount of 15%-30% cancer cases. As discussed, this is partly because p53 is one of the most important protein in cell biology and is implicated in wide range of disorders. For example, we have identified at least 4 of the 6 hotspot mp53s and a large number of non-hotspot mp53s to be efficiently rescuable by ATO and PANDA.

Our personalized treatment separates those patients suitable for treatments with PANDA Agent and those who are not. By selecting those patients with rescuable mp53, we can begin to treat patients based on p53 mutation rather than cancer type. It is known that different missense mutations will confer different activities to mp53 (Freed-Pastor and Prives, 2012), which can lead to different treatment outcomes in patients harboring different mp53s. Accordingly, others like us advocate tailoring treatments to the types of p53 mutations present rather than simply whether mp53 or wtp53 is present (Muller and Vousden, 2013, 2014). However, a compound that can effectively treat and rescue mp53 was not identified until now. Remarkably, our discoveries on the MDS patient-derived p53-S241F, p53-S241C as well as the other artificially generated p53 mutants on S241 support that PANDA Agents rescuing efficiency is determined not only by the p53 mutation site but also by the new residue generated (FIG. 26). Additionally, our results show that PANDA Agents can rescue de novo p53 mutations created by cancer treatment. Accordingly, our PANDA Agents can provide an important complementary treatment to other effective drugs for treating a p53 disorder, including cancer, thereby opening the possibility to use the side effects created by those drugs that are likely to also cause DNA mutation (and therefor p53 mutation) during treatment.

We have previously described a method of determining whether a mp53 is rescuable or not by IP or functional assays. However, these procedures must be done in a professional laboratory, and is time consuming and costly. The method of determining whether a mp53 is rescuable by determining whether a rescuable mp53 is present in the subject, as described herein, greatly improves the efficiency and financial burden for the subject.

In addition to use in humans, results from our animal studies also support using PANDA agent to treat a p53 disorder, such as cancer, for veterinary use, for example, in such as a mouse, dog, a cat, and other companion animals, a cattle and other livestock, a wolf, a panda bear, or other zoo animals, and a horse or other equines

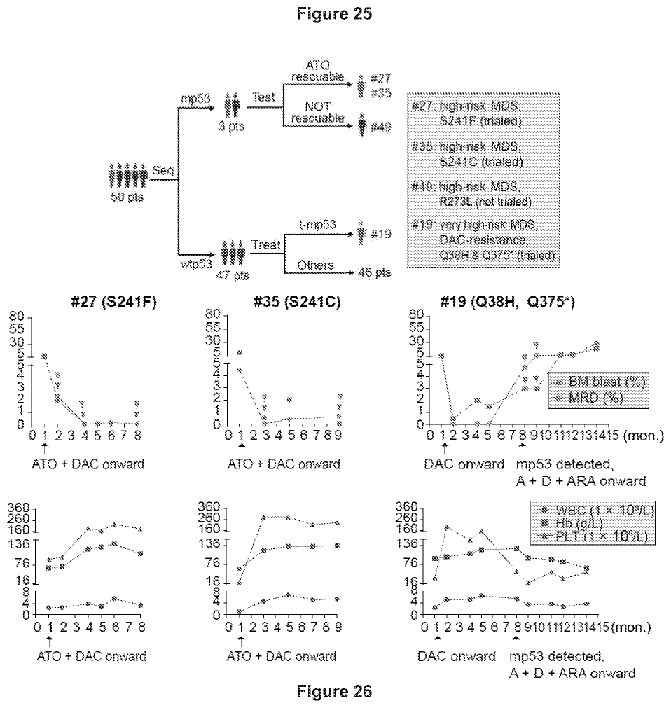

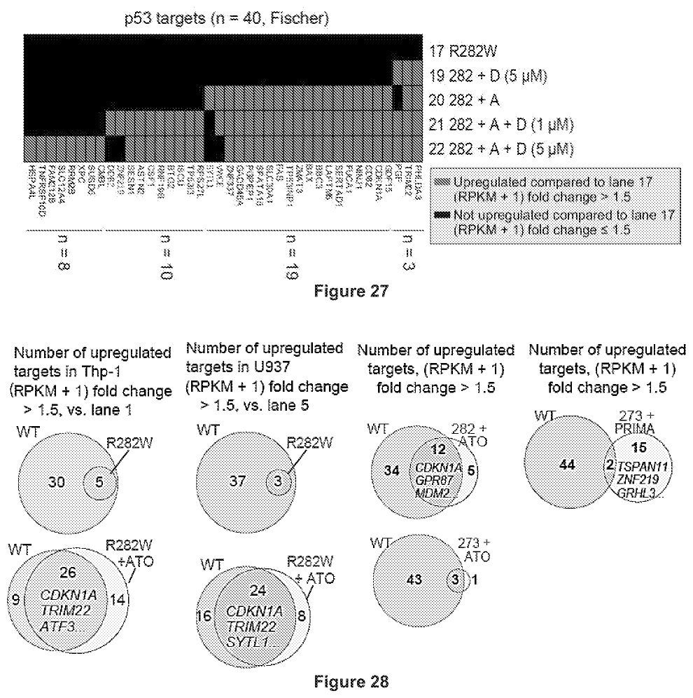

Additionally, we discovered that mp53 (for example, p53-R175H) and PANDA (for example, PANDA-R175H) responded differently to the DNA-damaging agents, such as Cisplatin, Etoposide, Adriamycin/Doxorubicin, 5-Fluorouracil, Cytarabine (ara-C), Azacitidine, and Decitabine(DAC), suggesting they may trigger distinctly treatment outcomes. We discovered Ser15, Ser37, and Lys382 were inertly modified on p53-R175H upon DNA-damaging treatment; however, they behave like wtp53 in that they are actively modified on PANDA-R175H upon DNA-damaging treatment (FIG. 25). We discovered Ser20 was inertly modified on p53-R175H irrespective of DNA-damaging stress; however it is actively modified on PANDA-R175H irrespective of DNA-damaging stress. These results suggest that p53-R175H and PANDA-R175H distinctly respond to therapies and thus may trigger distinctly treatment outcomes This also suggested the PANDA-R175H behave like wtp53 by being actively modified by DNA-damaging agents. These results support a synergetic combination method of treatment using a combination of a PANDA Agent and a DNA-damaging agent, such as DAC and ara-C, to treat a p53 disorder, such as a MDS patient with rescuable mp53. We further saw that the PANDA Agent As₂O₃ structurally and/or transcriptionally rescued a wide-spectrum of mp53s (Table 9), including other commonly occurring mp53s, such as p53-C176F, p53-H179R, and p53-Y220C; mp53s with contacting hotspots, such as mp53-R248Q; and mp53s with mutations outside of DNA-binding region, such as p53-V143A, p53-F270C, and p53-1232T (Table 9 and FIG. 12).

The characteristics of PANDA-forming reactions include the following:
(a) prefers to rescue Structural mp53;
(b) works for both human mp53 and mouse mp53;
(c) works in both mammalian cells and bacterial cells;
(d) works in vivo (in cells) and in vitro (in reaction buffer)
(e) mp53 cysteine(s) are involved;
(f) reaction is in a 1:1 molar ratio between mp53 and As atom
(g) direct reaction; and
(h) covalent reaction.

The characteristics of ATO mediated folding include:
(a) able to properly fold all tested Structural hotspot mp53s with a range of efficiency, including high to extremely high efficiency;
(b) instant folding (<15 min);
(c) folding is independent of cell types and treatment contexts, including resistant to EDTA in IP buffer;
(d) folding is much more efficient than any of the reported compounds;
(e) p53-R175H is almost fully restored as measured by the PAb1620 epitope;
(f) efficient for both human mp53 and mouse mp53;
(g) works in both mammalian cells and bacterial cells;
(h) can fold mp53 that has been previously unfolded;
(i) inhibits mp53 aggregation; and
(j) Cys135 and Cys141 are involved in As-mediated mp53 folding.

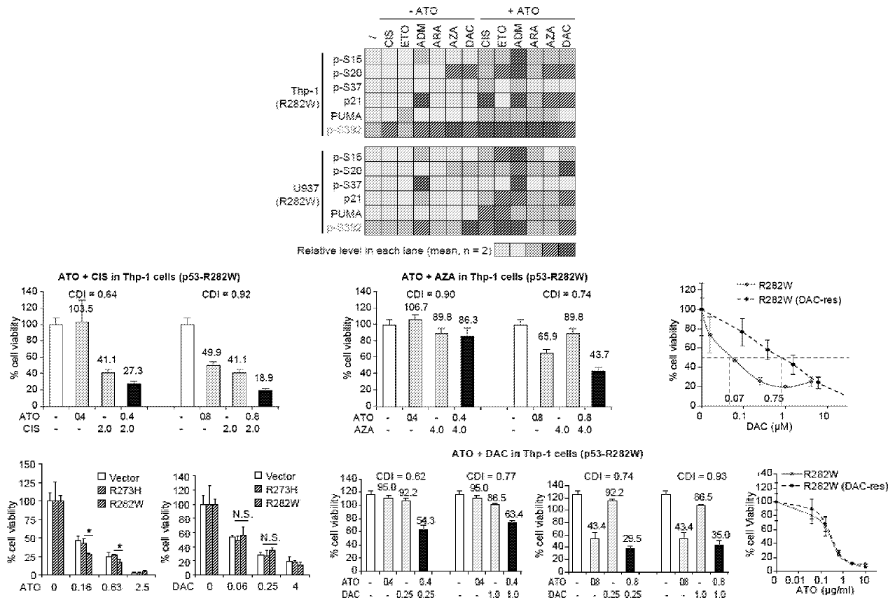

As disclosed herein, we discovered that (1) that ATO can function synergistically with other cancer inhibition therapies, (2) that combination anticancer therapy containing ATO has significant promises, and (3) that ATO may increase the efficacy of the wtp53-reactivating agents, such as MDM2 inhibitors, many of which are currently under clinical trials (FIG. 24)

### EXAMPLES

### 1.6 Plasmids, Antibodies, Cell Lines, Compounds, and Mice

pcDNA3.1 expressing human full length p53 was gift from Prof. Xin Lu (the University of Oxford), pGEX-2TK expressing fusion protein of GST and human full length p53 was purchased from Addgene (#24860), pET28a expressing p53 core was cloned for crystallization experiment without introducing any tag.

Primary antibodies were purchased from the following companies: DO1 (ab1101, Abcam), PAb1620 (MABE339, EMD Millipore), PAb240 (OP29, EMD Millipore), PAb246 (sc-100, Santa Cruz), PUMA (4976, Cell signaling), PIG3 (ab96819, Abcam), BAX (sc-493, Santa Cruz), p21 (sc-817, Santa Cruz), MDM2 (OP46-100UG, EMD Millipore), Biotin (ab19221, Abcam), Tubulin (ab11308, Abcam), β-actin (A00702, Genscript), p53-S15 (9284, Cell signaling), p53-S20 (9287, Cell signaling), p53-S37 (9289, Cell signaling), p53-S392 (9281, Cell signaling), p53-K382 (ab75754, Abcam), KU80 (2753, Cell signaling). CM5 antibody was gift from Prof. Xin Lu. HRP conjugated secondary antibody specifically reacts with light chain was from Abcam (ab99632).

H1299 and Saos-2 cell lines expressing null p53 was gift from Prof. Xin Lu. H1299 cell lines expressing tet-off regulated p53-R175H or tet-on regulated wtp53 were prepared as reported previously (Fogal et al., 2005). MEFs were prepared from E13.5 TP53-/- and TP53-R172H/R172H embryos. The other cell lines were obtained from ATCC.

Compounds were purchased from the following companies: DMSO (D2650, sigma), CP31398 (PZ0115, sigma), Arsenic trioxide (202673, sigma), STIMA-1 (506168, Merck Biosciences), SCH 529074 (4240, Tocris Bioscience), PhiKan 083 (4326, Tocris Bioscience), MiRA-1 (3362, Tocris Bioscience), Ellipticine (3357, Tocris Bioscience), NSC 319726 (S7149, selleck), PRIMA-1 (S7723, selleck), RITA (NSC 652287, S2781, selleck), Cycloheximide (C7698, sigma), Biotin (A600078, Sangon Biotech), Doxycycline hyclate (D9891, sigma), Cisplatin (CIS, P4394, sigma), Etoposide (ETO, E1383, sigma), Adriamycin (ADM, S1208, selleck), 5-Fluorouracil (5-FU, F6627, sigma), Cytarabine (ARA, S1648, selleck), Azacitidine (AZA, A2385, sigma), Decitabine (DAC, A3656, sigma). Bio-As and Bio-Dithi-As were gift from Kenneth L. Kirk (NIH; PMID: 18396406).

The TP53 wild-type mice, female nude mice and NOD/SCID mice were obtained from the Shanghai Laboratory Animal Center, Chinese Academy of Sciences. TP53-R172H/R172H mice were generated from the parent mice (026283) purchased from Jackson Lab. TP53−/− mice (002101) were purchased from National Resource Center of Model Mice of China.

DNA samples were sequenced in rainbow-genome technique Ltd (Shanghai) and Shanghai Biotechnology corporation (Shanghai).

### 1.7 Preparation of PANDA (without p53's N-Terminus and C-Terminus, without Tag) Formed in Bacteria

Constructions expressing recombinant TP53 core domain were transformed into E. coli strain BL21-Gold. Cells were cultured in either LB or M9 medium at 37° C. to mid-log phase. 0.5 mM isopropyl-p-D-thiogalactopyranoside (IPTG) was added in presence/absence of 50 μM As/Sb/Bi and 1 mM ZnCl₂ at 25° C. for overnight. Cells were harvested by centrifugation at 4 000 RPM for 20 minutes (˜10 g cell paste yielded from 1 liter of medium) and then sonicated in lysate buffer (50 mM Tris, pH 7.0, 50 mM NaCl, 10 mM DTT and 1 mM phenylmethylsulfonyl fluoride) in presence/absence of 50 μM As/Sb/Bi. Soluble lysate was loaded onto a SP-Sepharose cation exchange column (Pharmacia) and eluted with a NaCl gradient (0-1 M) then, if necessary, additionally purified by affinity chromatography with a heparin-Sepharose column (Pharmacia) in Tris.HCl, pH 7.0, 10 mM DTT with a NaCl gradient (0-1 M) for elution. Future purification was performed by gel-filtration using Superdex 75 column using standard procedure.

Processes after cell lysing are done at 4° C. Protein concentration was measured spectrophotometrically by using an extinction coefficient of 16 530 cm⁻¹M⁻¹ at 280 nm. All protein purification steps were monitored by 4-20% gradient SDS-PAGE to ensure they were virtually homogeneous.

### 1.8 Preparation of PANDA (with GST Tag) Formed in Bacteria

Constructions expressing GST-p53 (or GST-mp53) were transformed into E. coli strain BL21-Gold. Cells were grown in 800 ml LB medium at 37° C. to mid-log phase. 0.3 mM IPTG with/without 50 μM As/Sb/Bi was added at 16° C. for 24 h. Cells were harvested by centrifugation at 4 000 RPM for 20 minutes and then sonicated in 30 ml lysate buffer (58 mM Na2HPO4.12H2O, 17 mM NaH2 PO4.12H2O, 68 mM NaCl, 1% Triton X-100) in presence/absence of 50 μM As/Sb/Bi. Cell supernatant after 9000 RMP for 1 hour was added with 400 μl glutathione beads (Pharmacia) and incubated overnight. Beads were washed with lysate buffer for 3 times. Recombinant protein was then eluted by 300 μl elution buffer (10 mM GSH, 100 mM NaCl, 5 mM DTT and 50 mM Tris-HCl, pH 8.0). Processes after cell lysing are done at 4° C. All protein purification steps were monitored by 4-20% gradient SDS-PAGE to ensure they were virtually homogeneous.

### 1.9 Preparation of PANDA Formed in Insect Cells

Baculovirus infected Sf9 cells expressing recombinant human full-length p53 or p53 core in presence/absence of 50 μM As/Sb/Bi were harvested. They lysed in lysate buffer (50 mM Tris-HCl, pH 7.5, 5 mM EDTA, 1% NP-40.5 mM DTT, 1 mM PMSF, and 0.15 M NaCl) in presence/absence of 50 μM As/Sb/Bi. The lysates were then incubated on ice for 30 min, followed by centrifuging at 13000 rpm for 30 min. The supernatant was diluted 4-fold using 15% glycerol, 25 mM HEPES, pH 7.6, 0.1% Triton X-100, 5 mM DTT and 1 mM Benzamidine. They were further filtered using a 0.45 mm filter, and purified by Heparin-Sepharose column (Pharmacia). Purified protein was then concentrated using YM30 Centricon (EMD, Millipore). All protein purification steps were monitored by 4-20% gradient SDS-PAGE to ensure they were virtually homogeneous.

### 1.10 Preparation of PANDA Formed In Vitro

PANDA can be efficiently formed by mixing p53, either purified p53 or p53 in cell lysate, with one or more PANDA Agent. For example, in reaction buffer (20 mM HEPES, 150 mM NaCl, pH 7.5), we mixed purified recombinant p53 core and As/Sb/Bi compounds in a ratio ranging from 10:1-1:100 at 4° C. for overnight. The formed PANDA was then purified using dialysis to eliminate compounds.

### 1.11 In Vitro Reaction of Recombinant GST-p53-R175H and as

50 μM purified recombinant protein GST-p53-R175H in reaction buffer (10 mM GSH, 100 mM NaCl, 5 mM DTT and 50 mM Tris-HCl, pH 8.0) was added with Biotin-As to obtain arsenic to p53 molar ratio of either 10:1 or 1:1. The mixture solution was incubated at 4° C. for overnight and then divided into three parts. Each part was subjected to SDS-PAGE, followed by Coomassie blue staining (5 μg GST-p53-R175H applied), p53 immunoblotting (0.9 μg GST-p53-R175H applied) or Biotin immunoblotting (5 μg GST-p53-R175H applied), respectively.

### 1.12 Immunoprecipitation

For immunoprecipitation, mammalian cells or bacteria cells were harvested and lysed in NP40 buffer (50 mM Tris-HCl pH 8.0, 150 mM NaCl, 1% NP40) with cocktail of protease inhibitors (Roche Diagnostics). Cell lysates were then sonicated for 3 times, followed by spinning at 13,000 RPM for 20 min. Supernatant was adjusted to a final concentration of 1 mg/ml total protein using 450 μl NP40 buffer and incubated with 20 μl protein G beads and 1-3 μg corresponding primary antibody for 2 hr at 4° C. The beads were washed for three times with 20-25° C. NP40 buffer at room temperature. After spinning down, the beads were boiled for 5 min in 2×SDS loading buffer, followed by Western blotting.

### 1.13 Biotin-Arsenic Based Pull-Down Assay

Cells were treated with 4 μg/ml Bio-As or Bio-dithi-As for 2 hours. Cells were lysed in NP40 buffer (50 mM Tris-HCl pH 8.0, 150 mM NaCl, 1% NP40) with cocktail of protease inhibitors (Roche Diagnostics). Cell lysates were then sonicated for 3 times, followed by spinning at 13,000 RPM for 1 hr. Supernatant was adjusted to a final concentration of 1 mg/ml total protein using 450 μl NP40 buffer and incubated with 20 μl streptavidin beads for 2 hr at 4° C., followed by bead washing and Western blotting.

### 1.14 Biotin-DNA Based Pull-Down Assay

To prepare double-stranded oligonucleotides, equal amount of complementary single stranded oligonucleotides were heated at 80° C. for 5 min in 0.25 M NaCl, followed by slow cooling to room temperature. Sequences of single stranded oligonucleotides were followed:

| | |
|---|---|
| Consensus | 5′-Biotin-TCGAGAGGCATGTCTAGGCATGTCTC |
| PUMA | 5′-Biotin-CTGCAAGTCCTGACTTGTCC |
| PIG3 | 5′-Biotin-AGAGCCAGCTTGCCCACCCATGCTCGC |
| | GTG |
| BAX | 5′-Biotin-TCACAAGTTAAGACAAGCCTGGGCGTG |
| | GGC |
| MDM2 | 5′-Biotin-CGGAACGTGTCTGAACTTGACCAGCTC |
| p21 | 5′-Biotin-CGAGGAACATGTCCCAACATGTTGCTC |
| | GAG |
| Consensus-R | 5′-GAGACATGCCTAGACATGCCTCTCGA |
| PUMA-R | 5′-GGACAAGTCAGGACTTGCAG |
| PIG3-R | 5′-CACGCGAGCATGGGTGGGCAAGCTGGCTCT |
| BAX-R | 5′-GCCCACGCCCAGGCTTGTCTTAACTTGTGA |
| MDM2-R | 5′-GAGCTGGTCAAGTTCAGACACGTTCCG |
| p21-R | 5′-CTCGAGCAACATGTTGGGACATGTTCCTCG |

Cells were harvested and lysed in NP40 buffer (50 mM Tris-HCl pH 8.0, 150 mM NaCl, 1% NP40) with cocktail of protease inhibitors (Roche Diagnostics). Cell lysates were then sonicated for 3 times, followed by spinning at 13,000 RPM for 1 hr. Supernatant was adjusted to a final concentration of 1 mg/ml total protein using 450 μl NP40 buffer and incubated with 20 μl streptavidin beads (s-951, Invitrogen), 20 pmoles of biotinylated double-stranded oligonucleotides, and 2 μg of poly(dl-dC) (sc-286691, Santaz cruz). Lysates were incubated for 2 hr at 4° C., followed by bead washing and immunoblotting.

### 1.15 Immunoblotting

Immunoblotting was performed as reported previously (Lu et al., 2013).

### 1.16 Luciferase Assay

Cells were plated at a concentration of 2×10⁴ cells/well in 24-well plates, followed by transfection of luciferase reporter plasmids for 24 hr. All transfection contained 300 ng p53 expressing plasmid, 100 ng of luciferase reporter plasmid and 5 ng of renilla plasmid per well. After agent treatment, cells were lysed in luciferase reporter assay buffer and determined using a luciferase assay kit (Promega). Activities of luciferase were divided by that of renilla to normalize the transfection efficiency. For more details, see (Lu et al., 2013).

### 1.17 Colony Formation Assay

Treated cells were digested with trypsin. 100, 1000 or 10,000 cells/well were seeded in 12-well plates and kept in culture for 2-3 weeks. Fresh medium was replaced every three days.

### 1.18 Non-Denaturing PAGE

Cells were lysed in either CHAPS buffer (18 mM 3-[(3-cholamidopropyl) dimethylammonio]-1-propanesulfonic acid in TBS) or M-PER buffer (78501, Invitrogen) containing DNase and protease inhibitors for 15 min at 4° C. or 37° C. Cell lysate was added with 20% glycerol and 5 mM Coomassie G-250 before loading into 3-12% Novex Bis-Tris gradient gels. The electrophoresis was performed at 4° C. according to the manufacturer's instructions. Proteins were transferred onto the polyvinylidene fluoride membranes and fixed with 8% acetic acid for 20 min. The fixed membranes were then air dried and destained with 100% methanol. Membranes were blocked for overnight with 4% BSA in TBS at 4° C. before immunoblotting.

### 1.19 Real Time qPCR

Total RNA was isolated from cells using Total RNA Purification Kit (B518651, Sangon Biotech). 1 μg total RNA was reverse-transcribed using the GoScript®™ Reverse Transcriptase System (A5001, Promega) following manufacturer's protocol. PCR was performed in triplicate using SYBR green mix (Applied Biosystems), and a ViiA™ 7 Real-Time PCR System (Applied Biosystems) under the following conditions: 10 min at 95° C. followed by 40 cycles of 95° C. for 15 s and 60° C. for 1 min. Specificity of the PCR product was checked for each primer set and samples from the melting curve analysis. Expression levels of targeted genes were normalized relative to levels of β-actin adopting comparative Ct method. The primer sequences are as follows: MDM2 forward 5′-CCAGGGCAGCTACGGTTTC-3′, reverse 5′-CTCCGTCATGTGCTGTGACTG-3′; PIG3 forward 5′-CGCTGAAATTCACCAAAGGTG-3′, reverse 5′-AACCCATCGACCATCAAGAG-3′; PUMA forward 5′-ACGACCTCAACGCACAGTACG-3′, reverse 5′-TCCCATGATGAGATTGTACAGGAC-3′; p21 forward 5′-GTCTTGTACCCTTGTGCCTC-3′, reverse 5′-GGTAGAAATCTGTCATGCTGG-3′; Bax forward 5′-GATGCGTCCACCAAGAAGCT-3′, reverse 5′-CGGCCCCAGTTGAAGTTG-3′; #-actin forward 5′-ACTTAGTTGCGTTACACCCTTTCT-3′, reverse 5′-GACTGCTGTCACCTTCACCGT-3′.

### 1.20 Xenograft Assay

H1299 xenograft. H1299 cells expressing tet-off regulated p53-R175H (1*10⁶ cells) suspended in 100 μl saline solution were subcutaneously injected into the flanks of 8-9 weeks old female nude mice. When the tumor area reached 0.1 cm (day 1), 5 mg/kg ATO were intraperitoneally injected 6 consecutive days per week. In DOX groups, 0.2 mg/ml doxycycline was added to drinking water. Tumor size was measured every 3 days with vernier callipers. Tumor volumes were calculated using the following formula: (L*W* W)/2, in which L represents the large diameter of the tumor, and W represents the small diameter. When tumor area reached −1 cm diameter in any group, mice were sacrificed and isolated tumors were weighed. The analysis of the differences between the groups was performed by Two-way RM ANOVA with Bonferroni correction.

CEM-C1 xenograft. 8-9 week old NOD/SCID mice were intravenously injected through the tail vein with 1*10⁷ cells of CEM-C1 T-ALL cells (day 1). After engraftment, peripheral blood samples were obtained from the mice retro-orbital sinus every 3 or 4 days from day 16 to day 26. Residual red blood cells were removed using erythrocyte lysis buffer (NH₄Cl 1.5 mM, NaHCO₃ 10 Mm, EDTA-2Na 1 mM). The isolated cells were double stained with PerCP-Cy5.5-conjugated anti-mouse CD45 (mCD45) (BD Pharmigen™, San Diego, Calif.) and FITC-conjugated anti-human CD45 (hCD45) (BD Pharmigen™, San Diego, Calif.) antibodies before flow cytometric analysis conducted. When the percent of hCD45+ cells in peripheral blood reached 0.1% one mice (day 22), ATO was prepared for injection. On day 23, 5 mg/kg ATO were intravenously injected via tail-vein in 0.1 ml saline solution 6 consecutive days per week. The comparison of the hCD45+ cells percent between the groups was performed by unpaired t test. The life-span of mice was analyzed by Log-rank (Mantel-Cox) test.

All statistical analysis was performed using GraphPad Prism 6.00 for Windows (La Jolla Calif., USA). The animals were housed in specific pathogen-free conditions. Experiments were carried out according to the National Institutes of Health Guide for Care and Use of Laboratory Animals.

### 1.21 Statistical Analysis

Statistical analysis was carried out using Fisher's exact test (two-tailed) unless otherwise indicated. p values less than 0.05 were considered statistically significant unless otherwise indicated.

TABLE 1
1.22 1100 three-valence arsenic (“As”) containing compounds were predicted to efficiently bind PANDA
Pock6jet and efficiently rescue Structural mp53. All of the 94.2 million structures recorded in PubChem
(https://pubchem.ncbi.nimmih.gov/) were applied for 4C+ screening. In the 4C+ screening, we collected those
with more than 2 cysteine-binding potential. Carbon-binding As/Sb/Bi bond has defect in binding
cysteine since this bond cannot be hydrolyzed. The other Asi/Sb/Bi bond can be
hydrolyzed in cells and thus is able to bind cysteine.Arsenic 3 Valence Panda Agents
Arsenic 3 Valence PANDA Agents

| CID No. | CID No. | CID No. | CID No. | CID No. | CID No. | CID No. | CID No. | CID No. |
|---|---|---|---|---|---|---|---|---|
| 544 | 545 | 14,770 | 14,772 | 19,167 | 19,541 | 21,763 | 23,969 | 24,569 |
| 24,570 | 24,571 | 24,575 | 24,577 | 24,679 | 24,967 | 25,130 | 25,156 | 25,214 |
| 26,435 | 33,719 | 40,516 | 48,705 | 61,545 | 61,738 | 62,222 | 62,391 | 76,591 |
| 78,450 | 82,779 | 82,780 | 82,781 | 82,782 | 82,783 | 82,784 | 82,785 | 82,786 |
| 82,787 | 82,876 | 82,877 | 82,873 | 82,879 | 82,880 | 82,998 | 82,999 | 83,000 |
| 83,001 | 83,002 | 83,003 | 84,670 | 89,859 | 91,500 | 96,373 | 109,033 | 116,252 |
| 116,256 | 116,257 | 117,908 | 122,957 | 138,780 | 139,279 | 139,298 | 139,321 | 139,322 |
| 139,323 | 139,324 | 139,773 | 139,891 | 139,939 | 140,012 | 143,005 | 145,013 | 150,167 |
| 158,274 | 159,390 | 165,514 | 167,244 | 167,245 | 167,445 | 178,411 | 178,412 | 178,413 |
| 178,414 | 178,415 | 178,416 | 180,503 | 184,133 | 187,781 | 193,333 | 197,119 | 204,633 |
| 602,148 | 606,254 | 607,452 | 635,386 | 2,724,938 | 2,784,590 | 3,019,767 | 3,019,768 | 3,033,858 |
| 3,051,241 | 3,051,242 | 3,051,637 | 3,051,638 | 3,055,940 | 3,083,023 | 3,627,253 | 4,093,503 | 4,390,915 |
| 4,628,691 | 5,127,350 | 5,148,939 | 5,149,849 | 5,231,667 | 5,245,643 | 5,246,853 | 5,247,035 | 5,247,036 |
| 5,247,256 | 5,247,881 | 5,248,768 | 5,248,769 | 5,255,179 | 5,256,195 | 5,256,504 | 5,258,395 | 5,258,981 |
| 5,259,398 | 5,259,672 | 5,460,506 | 5,460,562 | 5,460,564 | 5,460,565 | 5,460,566 | 5,460,587 | 5,460,722 |
| 5,491,620 | 5,743,819 | 5,743,820 | 6,284,547 | 6,326,784 | 6,327,292 | 6,329,460 | 6,329,630 | 6,329,663 |
| 6,331,458 | 6,334,574 | 6,335,843 | 6,337,007 | 6,391,215 | 6,395,537 | 6,395,538 | 6,395,543 | 6,395,544 |
| 6,395,864 | 6,395,865 | 6,395,866 | 6,395,867 | 6,395,868 | 6,397,860 | 6,398,629 | 6,711,222 | 6,857,430 |
| 6,857,431 | 6,857,580 | 6,914,516 | 6,914,518 | 6,914,531 | 9,548,861 | 9,543,868 | 9,548,869 | 9,553,924 |
| 9,553,925 | 9,823,432 | 9,940,355 | 10,285,774 | 10,540,385 | 10,709,168 | 10,714,189 | 10,716,984 | 10,717,398 |
| 10,744,851 | 10,752,266 | 10,886,649 | 10,887,184 | 10,887,185 | 10,896,360 | 10,908,016 | 10,930,378 | 10,963,436 |
| 11,010,611 | 11,010,612 | 11,017,329 | 11,017,851 | 11,017,852 | 11,049,547 | 11,134,592 | 11,148,325 | 11,193,862 |
| 11,248,058 | 11,251,274 | 11,256,472 | 11,298,875 | 11,310,398 | 11,316,887 | 11,385,570 | 11,402,381 | 11,412,921 |
| 11,431,015 | 11,465,219 | 11,476,512 | 11,562,561 | 11,607,348 | 11,650,722 | 11,676,828 | 11,686,352 | 11,706,542 |
| 11,751,340 | 11,831,174 | 11,966,301 | 11,966,302 | 11,968,269 | 11,970,814 | 11,970,815 | 11,980,559 | 12,022,729 |
| 12,029,075 | 12,060,026 | 12,060,027 | 12,060,028 | 12,060,029 | 12,062,780 | 12,062,953 | 12,062,954 | 12,105,505 |
| 12,549,303 | 12,549,305 | 12,549,308 | 12,622,637 | 12,639,434 | 12,690,546 | 12,690,547 | 12,692,939 | 12,754,317 |
| 12,754,318 | 12,758,717 | 12,758,718 | 12,758,720 | 12,909,455 | 12,946,537 | 12,946,538 | 12,964,989 | 12,970,646 |
| 13,068,571 | 13,076,527 | 13,426,139 | 13,464,647 | 13,528,538 | 13,528,542 | 13,528,544 | 13,528,552 | 13,710,813 |
| 13,726,197 | 13,751,567 | 14,042,655 | 14,226,398 | 14,389,419 | 14,420,822 | 14,951,535 | 14,962,238 | 15,130,450 |
| 15,165,101 | 15,193,599 | 15,193,603 | 15,214,107 | 15,241,817 | 15,393,356 | 15,443,371 | 15,43,375 | 15,452,707 |
| 15,452,708 | 15,452,709 | 15,463,897 | 15,544,525 | 15,763,917 | 15,768,782 | 15,770,833 | 15,770,922 | 15,773,246 |
| 15,775,979 | 15,779,670 | 15,784,977 | 15,787,963 | 15,787,964 | 15,793,134 | 15,801,408 | 15,804,634 | 15,831,253 |
| 15, 831,254 | 15,836,478 | 15,836,479 | 15,836,480 | 15,845,941 | 15,902,288 | 15,909,316 | 15,911,001 | 16,103,935 |
| 16, 122,615 | 16,122,617 | 16,217,311 | 16,687,467 | 16,687,468 | 16,688,418 | 16,688,647 | 16,688,985 | 17,950,332 |
| 17,972,457 | 17,972,458 | 17,999,318 | 18,620,370 | 18,699,306 | 18,764,508 | 18,924,269 | 18,982,511 | 19,067,450 |
| 19,076,933 | 19,377,136 | 19,770,611 | 19,827,518 | 19,882,979 | 19,977,769 | 19,977,790 | 20,194,853 | 20,194,857 |
| 20,209,387 | 20,209,339 | 20,313,265 | 20,363,283 | 20,386,190 | 20,481,422 | 20,498,187 | 20,519,997 | 20,519,998 |
| 20,745,926 | 20,838,028 | 20,843,076 | 20,843,447 | 20,843,448 | 20,846,387 | 20,846,388 | 20,976,124 | 21,096,828 |
| 21,117,048 | 21,119,276 | 21,119,939 | 21,119,940 | 21,124,621 | 21,126,002 | 21,126,578 | 21,127,708 | 21,152,268 |
| 21,153,195 | 21,153,196 | 21,396,696 | 21,402,593 | 21,498,356 | 21,597,738 | 21,597,741 | 21,666,086 | 21,679,197 |
| 21,679,198 | 21,718,489 | 21,719,691 | 21,720,503 | 21,732,336 | 21,732,337 | 21,732,338 | 21,732,339 | 21,732,340 |
| 21,732,341 | 21,732,582 | 21,732,691 | 21,732,985 | 21,854,934 | 21,854,935 | 21,854,936 | 21,854,937 | 21,854,938 |
| 21,854,939 | 21,854,940 | 21,854,941 | 21,854,942 | 21,854,943 | 21,854,944 | 21,854,945 | 21,854,946 | 21,854,947 |
| 21,854,948 | 21,854,949 | 21,896,088 | 21,932,415 | 21,954,723 | 21,998,317 | 22,099,016 | 22,119,650 | 22,223,246 |
| 22,239,183 | 22,341,918 | 22,413,077 | 22,717,370 | 22,333,492 | 22,895,349 | 23,028,044 | 23,234,053 | 23,234,054 |
| 23,235,595 | 23,235,987 | 23,261,952 | 23,261,953 | 23,261,954 | 23,261,955 | 23,261,956 | 23,261,957 | 23,261,958 |
| 23,261,959 | 23,261,960 | 23,261,961 | 23,261,962 | 23,261,963 | 23,261,964 | 23,261,965 | 23,261,966 | 23,261,967 |
| 23,261,968 | 23,262,012 | 23,262,763 | 23,263,174 | 23,263,178 | 23,263,198 | 23,263,199 | 23,263,239 | 23,270,086 |
| 23,280,718 | 23,354,875 | 23,412,685 | 23,412,687 | 23,412,683 | 23,412,690 | 23,412,691 | 23,412,692 | 23,412,693 |
| 23,412,694 | 23,412,695 | 23,412,696 | 23,412,697 | 23,412,725 | 23,412,739 | 23,412,743 | 23,416,402 | 23,418,415 |
| 23,419,990 | 23,420,083 | 23,424,003 | 23,424,004 | 23,443,680 | 23,588,764 | 23,538,768 | 23,616,631 | 23,616,677 |
| 23,618,364 | 23,618,423 | 23,613,916 | 23,618,935 | 23,618,936 | 23,513,946 | 23,620,087 | 23,620,337 | 23,620,895 |
| 23,620,896 | 23,620,897 | 23,623,772 | 23,665,003 | 23,663,346 | 23,696,518 | 23,697,313 | 23,719,535 | 23,719,541 |
| 23,719,552 | 24,733,960 | 24,846,178 | 24,834,190 | 25,113,239 | 25,147,458 | 25,147,462 | 25,147,473 | 25,208,316 |
| 42,615,748 | 42,627,489 | 42,627,490 | 42,627,491 | 42,627,492 | 44,144,454 | 44,151,659 | 44,152,997 | 44,153,294 |
| 44,395,086 | 44,563,905 | 45,051,723 | 45,479,364 | 45,479,365 | 45,479,524 | 45.479,525 | 49,866,861 | 50,922,181 |
| 50,922,452 | 50,931,488 | 50,931,515 | 50,931,522 | 50,933,634 | 50,933,999 | 50,934,000 | 50,936,983 | 53,239,239 |
| 53,249,217 | 53,339,027 | 53,339,028 | 53,349,346 | 53,349,347 | 53,349,348 | 53,349,349 | 53,349,350 | 53,349,351 |
| 53,349,352 | 53,423,860 | 53,432,730 | 53,471,870 | 53,492,526 | 54,603,741 | 54,611,691 | 54,696,380 | 54,723,496 |
| 56,840,839 | 57,346,053 | 57,346,345 | 57,346,984 | 57,347,790 | 57,351,126 | 57,351,127 | 57,354,213 | 57,354,214 |
| 57,359,120 | 57,359,121 | 57,376,611 | 57,376,762 | 57,448,761 | 57,448,769 | 57,448,813 | 57,448,860 | 57,466,141 |
| 57,467,921 | 57,484,655 | 57,517,166 | 57,763,376 | 57,763,404 | 58,743,125 | 58,857,119 | 59,080,074 | 60,207,945 |
| 71,301,049 | 71,309,374 | 71,310,805 | 71,310,806 | 71,334,864 | 71,336,057 | 71,339,387 | 71,345,105 | 71,349,031 |
| 71,350,771 | 71,350,783 | 71,353,378 | 71,353,379 | 71,353,399 | 71,355,900 | 71,355,927 | 71,357,772 | 71,358,656 |
| 71,358,665 | 71,358,778 | 71,359,001 | 71,359,074 | 71,361,408 | 71,361,450 | 71,363,675 | 71,367,036 | 71,367,054 |
| 71,367,078 | 71,367,094 | 71,367,112 | 71,367,851 | 71,370,608 | 71,377,166 | 71,377,167 | 71,380,410 | 71,386,575 |
| 71,389,205 | 71,389,206 | 71,389,207 | 71,389,208 | 71,395,187 | 71,395,375 | 71,396,322 | 71,401,400 | 71,401,410 |
| 71,407,586 | 71,410,644 | 71,410,645 | 71,417,968 | 71,417,969 | 71,430,842 | 71,436,452 | 71,443,407 | 71,443,408 |
| 71,446,592 | 71,446,618 | 71,446,960 | 71,526,741 | 71,580,995 | 71,586,773 | 72,720,419 | 73,351,195 | 73,357,742 |
| 73,415,825 | 73,894,232 | 73,894,234 | 74,765,958 | 74,935,276 | 76,871,762 | 76,963,989 | 76,966,440 | 78,060,866 |
| 78,061,006 | 78,062,686 | 78,063,158 | 78,063,159 | 78,066,382 | 78,066,392 | 78,070,72l | 85,571,114 | 85,577,990 |
| 85,605,969 | 85,607,971 | 85,609,099 | 85,612,664 | 85,613,640 | 85,632,096 | 85,756,556 | 85,775,826 | 85,779,417 |
| 85,792,258 | 85,793,306 | 85,808,862 | 85,824,116 | 85,850,779 | 85,850,780 | 85,850,781 | 85,850,782 | 85,850,783 |
| 85,850,785 | 85,850,786 | 85,850,789 | 85,850,790 | 85,860,792 | 85,884,398 | 85,911,906 | 85,977,561 | 85,994,471 |
| 86,013,960 | 86,014,167 | 86,065,878 | 86,126,557 | 86,154,041 | 86,159,344 | 86,172,918 | 86,176,315 | 86,205,935 |
| 86,249,175 | 86,717,687 | 87,090,222 | 87,094,975 | 87,109,955 | 87,111,979 | 87,118,088 | 87,144,977 | 87,144,978 |
| 87,189,618 | 87,205,247 | 87,205,248 | 87,234,713 | 87,246,827 | 87,248,447 | 87,255,851 | 87,255,859 | 87,255,872 |
| 87,255,873 | 87,235,874 | 87,255,878 | 87,255,879 | 87,255,885 | 87,255,886 | 87,410,957 | 87,472,810 | 87,472,812 |
| 87,473,759 | 87,474,264 | 87,474,374 | 87,474,643 | 87,474,789 | 87,475,307 | 87,475,822 | 87,475,824 | 87,475,926 |
| 87,476,165 | 87,476,184 | 87,476,290 | 87,476,403 | 87,476,530 | 87,476,558 | 87,476,21 | 87,476,845 | 87,476,848 |
| 87,477,042 | 87,477,044 | 87,477,165 | 87,477,318 | 87,477,537 | 37,477,756 | 87,477,757 | 87,478,031 | 87,434,462 |
| 87,551,850 | 87,562,825 | 87,581,052 | 87,581,053 | 87,676,186 | 87,700,226 | 87,700,230 | 87,700,407 | 87,776,602 |
| 87,812,518 | 87,858,508 | 87,980,104 | 87,980,186 | 87,989,328 | 88,144,816 | 88,162,162 | 88,184,704 | 88,248,144 |
| 88,263,967 | 88,266,006 | 88,305,231 | 88,305,232 | 88,315,700 | 88,342,746 | 88,389,746 | 88,415,561 | 88,433,797 |
| 88,438,798 | 88,464,855 | 88,464,856 | 88,491,208 | 88,510,137 | 88,510,138 | 88,510,139 | 88,510,140 | 88,513,649 |
| 88,513,650 | 88,513,651 | 88,625,918 | 88,625,919 | 88,633,062 | 88,658,127 | 88,686,405 | 88,720,065 | 88,738,074 |
| 88,738,075 | 88,738,076 | 88,738,077 | 88,738,154 | 88,744,197 | 88,791,225 | 88,798,792 | 88,798,793 | 88,807,785 |
| 88,814,284 | 88,838,825 | 88,838,826 | 89,525,461 | 89,588,457 | 89,921,988 | 90,115,830 | 90,470,710 | 90,479,326 |
| 91,733,954 | 91,750,137 | 91,750,137 | 91,751,251 | 91,886,304 | 91,988,436 | 91,989,816 | 91,994,839 | 91,999,513 |
| 91,999,513 | 92,001,091 | 92,001,092 | 92,002,930 | 92,003,357 | 92,007,238 | 92,024,388 | 92,024,388 | 92,024,399 |
| 92,024,401 | 92,024,402 | 92,025,101 | 92,025,102 | 92,025,103 | 92,025,104 | 92,025,105 | 92,025,106 | 92,025,107 |
| 92,025,108 | 92,025,109 | 92,025,110 | 92,025,111 | 92,025,112 | 92,025,113 | 92,025,732 | 92,026,266 | 92,026,424 |
| 92,026,427 | 92,026,688 | 92,026,716 | 92,026,737 | 92,026,781 | 92,026,881 | 92,026,963 | 92,027,058 | 92,027,110 |
| 92,027,269 | 92,027,367 | 92,027,458 | 92,027,511 | 92,027,541 | 92,027,722 | 92,028,182 | 92,028,237 | 92,028,421 |
| 92,028,660 | 92,028,686 | 92,028,728 | 92,028,791 | 92,028,795 | 92,028,877 | 92,028,881 | 92,028,938 | 92,043,202 |
| 100,918,999 | 100,919,000 | 100,942,061 | 100,942,062 | 100,949,123 | 100,986,766 | 100,986,767 | 100,991,044 | 100,931,045 |
| 100,999,027 | 101,005,335 | 101,005,336 | 101,005,337 | 101,005,338 | 101,005,339 | 101,013,781 | 101,013,782 | 101,013,783 |
| 101,047,156 | 101,064,325 | 101,085,742 | 101,113,845 | 101,113,846 | 101,116,387 | 101,117,156 | 101,118,991 | 101,174,827 |
| 101,237,011 | 101,260,419 | 101,282,796 | 101,289,874 | 101,354,116 | 101,354,274 | 101,379,755 | 101,411,205 | 101,436,717 |
| 101,436,720 | 101,458,326 | 101,459,329 | 101,485,971 | 101,485,973 | 101,485,974 | 101,499,194 | 101,499,195 | 101,533,802 |
| 101,533,803 | 101,547,226 | 101,575,977 | 101,617,225 | 101,619,265 | 101,666,991 | 101,666,992 | 101,682,434 | 101,682,435 |
| 101,682,436 | 101,682,437 | 101,682,438 | 101,694,974 | 101,694,975 | 101,694,976 | 101,694,977 | 101,694,978 | 101,694,979 |
| 101,695,476 | 101,763,094 | 101,763,095 | 101,763,095 | 101,763,096 | 101,763,096 | 101,763,097 | 102,098,990 | 102,167,962 |
| 101,838,903 | 101,838,904 | 101,848,513 | 101,852,195 | 101,861,498 | 101,861,499 | 101,913,584 | 101,923,897 | 101,948,144 |
| 102,015,291 | 102,034,400 | 102,062,180 | 102,062,402 | 102,063,353 | 102,076,551 | 102,076,552 | 102,095,097 | 102,098,989 |
| 102,098,990 | 102,167,962 | 102,180,144 | 102,180,145 | 102,186,306 | 102,213,935 | 102,213,936 | 102,213,937 | 102,226,697 |
| 102,239,094 | 102,351,891 | 102,351,892 | 102,351,897 | 102,351,898 | 102,371,199 | 102,414,495 | 102,414,496 | 102,415,253 |
| 102,496,603 | 102,499,917 | 102,499,918 | 102,511,469 | 102,528,387 | 102,528,388 | 102,528,389 | 102,528,390 | 102,593,935 |
| 102,601,572 | 119,077,607 | 122,364,373 | 122,378,045 | 122,378,046 | 123,133,548 | 123,133,548 | 129,628,305 | 129,629,427 |
| 129,630,515 | 129,631,280 | 129,631,344 | 129,631,597 | 129,631,743 | 129,631,758 | 129,631,787 | 129,631,891 | 129,634,318 |
| 129,635,174 | 129,637,470 | 129,642,045 | 129,642,046 | 129,643,934 | 129,643,936 | 129,643,938 | 129,645,426 | 129,651,528 |
| 129,652,225 | 129,655,074 | 129,659,469 | 129,660,712 | 129,672,983 | 129,677,715 | 129,679,346 | 129,679,347 | 129,679,425 |
| 129,679,623 | 129,680,077 | 129,686,362 | 129,639,790 | 129,692,591 | 129,693,147 | 129,703,372 | 129,703,373 | 129,705,075 |
| 129,705,678 | 129,715,852 | 129,724,489 | 129,729,547 | 129,729,551 | 129,737,952 | 129,741,926 | 129,742,025 | 129,742,535 |
| 129,756,855 | 129,757,023 | 129,757,215 | 129,774,469 | 129,774,997 | 129,805,810 | 129,809,454 | 129,821,735 | 129,828,133 |
| 129,828,662 | 129,828,842 | 129,828,911 | 129,829,589 | 129,842,137 | 129,842,236 | 129,842,237 | 129,849,818 | 129,858,925 |
| 129,858,931 | 129,863,921 | 129,865,429 | 129,365,431 | 129,873,049 | 129,887,306 | 129,388,984 | 129,889,538 | 129,890,164 |
| 129,891,119 | 129,891,127 | 129,891,128 | 129,892,162 | 131,700,384 | 131,736,786 | 131,852,787 | 131,852,788 | 131,864,457 |
| 131,864,465 | 131,364,960 | 131,865,018 | 131,871,206 | 131,872,531 | 131,874,138 | 131,874,746 | 131,876,990 | 131,876,991 |
| 131,876,992 | 131,884,149 | | | | | | | |

TABLE 2
1.23 3071 five-valence arsenic (“As”) containing compounds were predicted to efficiently bind PANDA Pocket and efficiently rescue structural
mp53. All of the 94.2 million structures recorded in PubChem (https://pubchem.ncbi.nlm.nih.gov/) were applied for 4C+ screening. In the 4C+
screening, we collected those with more than 2 cysteine-binding potential. Carbon-binding As/Sb/Bi bond has defect in binding cysteine since
this bond cannot be hydrolyzed. The other As/Sb/Bi bond can be hydrolyzed in cells and thus is able to bind cysteine.
Arsenic 5 Valence Panda Agents

| CID No. | CID No. | CID No. | CID No. | CID No. | CID No. | CID No. | CID No. | CID No. |
|---|---|---|---|---|---|---|---|---|
| 233 | 234 | 1,985 | 2,513 | 5,104 | 7,365 | 7,373 | 7,389 | 8,312 |
| 8,480 | 8,802 | 8,947 | 8,948 | 9,313 | 9,314 | 9,525 | 10,799 | 10,809 |
| 10,810 | 11,453 | 12,055 | 13,127 | 13,479 | 14,771 | 15,465 | 16,555 | 16,851 |
| 17,845 | 20,770 | 21,074 | 21,250 | 21,251 | 21,252 | 21,253 | 21,511 | 21,548 |
| 21,549 | 22,193 | 22,273 | 22,773 | 22,874 | 23,060 | 23,129 | 23,514 | 23,515 |
| 23,749 | 24,262 | 24,500 | 24,501 | 24,572 | 24,574 | 24,938 | 24,943 | 25,303 |
| 25,929 | 26,065 | 27,052 | 27,401 | 27,817 | 30,852 | 30,853 | 30,854 | 32,602 |
| 36,829 | 38,223 | 44,902 | 45,720 | 46,313 | 46,681 | 46,683 | 46,684 | 46,685 |
| 46,686 | 46,840 | 47,275 | 52,340 | 52,503 | 52,504 | 52,505 | 52,506 | 52,949 |
| 52,950 | 52,951 | 52,952 | 52,953 | 52,954 | 61,460 | 61,477 | 61,617 | 61,618 |
| 61,619 | 62,043 | 66,826 | 66,944 | 66,945 | 67,177 | 67,178 | 67,782 | 68,198 |
| 68,199 | 68,532 | 68,716 | 72,905 | 73,161 | 74,471 | 77,393 | 77,588 | 77,589 |
| 79,424 | 79,425 | 79,502 | 79,769 | 79,849 | 79,871 | 80,448 | 80,568 | 80,894 |
| 81,422 | 82,223 | 82,228 | 84,776 | 93,839 | 94,822 | 94,911 | 94,933 | 95,017 |
| 95,018 | 95,019 | 95,452 | 95,473 | 104,785 | 138,903 | 113,151 | 113,152 | 114,800 |
| 115,320 | 115,371 | 116,251 | 116,491 | 116,504 | 119,656 | 120,177 | 124,738 | 124,739 |
| 124,921 | 126,669 | 127,752 | 128,033 | 129,501 | 135,804 | 135,805 | 135,806 | 145,014 |
| 145,125 | 151,614 | 151,615 | 151,637 | 156,005 | 159,810 | 160,269 | 160,801 | 161,792 |
| 165,585 | 166,814 | 166,815 | 166,826 | 166,828 | 166,835 | 167,250 | 167,617 | 167,906 |
| 169,458 | 173,497 | 174,008 | 174,227 | 176,957 | 177,152 | 177,517 | 177,518 | 177,719 |
| 178,410 | 178,417 | 178,418 | 182,380 | 185,520 | 187,828 | 187,863 | 187,864 | 189,991 |
| 189,992 | 190,282 | 192,901 | 193,598 | 193,853 | 196,505 | 197,032 | 197,906 | 201,395 |
| 202,213 | 202,581 | 202,583 | 203,113 | 203,535 | 203,784 | 219,617 | 220,597 | 220,599 |
| 220,807 | 222,574 | 223,334 | 223,335 | 223,336 | 223,337 | 223,338 | 223,339 | 223,342 |
| 223,343 | 223,344 | 223,345 | 223,347 | 223,349 | 223,352 | 223,788 | 223,789 | 223,806 |
| 223,807 | 224,248 | 224,249 | 224,250 | 224,251 | 224,252 | 224,253 | 224,255 | 224,256 |
| 224,257 | 224,258 | 224,260 | 224,261 | 224,262 | 224,263 | 224,264 | 224,265 | 224,267 |
| 224,268 | 224,270 | 224,271 | 224,272 | 224,273 | 224,274 | 224,275 | 224,276 | 224,278 |
| 224,282 | 224,283 | 224,284 | 224,288 | 224,289 | 224,290 | 224,294 | 224,295 | 224,298 |
| 224,301 | 224,309 | 224,575 | 224,660 | 224,877 | 224,878 | 224,880 | 224,881 | 224,891 |
| 224,892 | 224,893 | 224,894 | 224,896 | 224,897 | 224,901 | 224,902 | 224,903 | 224,906 |
| 224,907 | 224,908 | 224,911 | 224,912 | 224,914 | 224,916 | 224,917 | 224,918 | 224,920 |
| 224,921 | 224,922 | 224,923 | 224,924 | 224,925 | 224,927 | 224,928 | 224,929 | 225,780 |
| 225,783 | 225,785 | 225,786 | 225,788 | 225,789 | 225,791 | 225,798 | 225,799 | 225,802 |
| 225,806 | 229,965 | 231,057 | 231,882 | 231,895 | 231,896 | 231,966 | 231,967 | 231,968 |
| 231,969 | 232,560 | 232,563 | 232,565 | 232,566 | 232,567 | 233,201 | 233,281 | 233,320 |
| 233,321 | 233,324 | 233,325 | 233,833 | 234,155 | 234,202 | 234,284 | 234,285 | 234,286 |
| 234,535 | 234,536 | 234,539 | 234,557 | 234,559 | 234,560 | 234,637 | 234,639 | 234,890 |
| 236,817 | 237,668 | 237,669 | 237,670 | 237,671 | 237,672 | 237,673 | 237,674 | 237,991 |
| 238,189 | 238,195 | 238,219 | 233,220 | 238,221 | 238,223 | 238,243 | 238,244 | 239,021 |
| 239,026 | 239,027 | 239,028 | 239,032 | 240,762 | 241,152 | 241,153 | 241,156 | 241,157 |
| 241,158 | 241,631 | 241,636 | 241,906 | 241,907 | 241,908 | 241,909 | 241,911 | 241,912 |
| 241,913 | 241,918 | 241,958 | 241,963 | 241,964 | 2,4,263 | 244,264 | 244,266 | 247,798 |
| 247,799 | 247,800 | 251,060 | 252,526 | 256,154 | 258,099 | 260,823 | 260,832 | 261,003 |
| 261,005 | 261,009 | 261,010 | 261,011 | 261,012 | 261,013 | 261,014 | 261,015 | 261,016 |
| 261,020 | 261,021 | 261,022 | 261,023 | 261,024 | 261,025 | 261,027 | 261,032 | 261,033 |
| 261,034 | 261,035 | 261,041 | 261,042 | 261,044 | 261,045 | 261,049 | 261,050 | 261,227 |
| 261,292 | 263,033 | 266,086 | 266,087 | 266,088 | 266,089 | 266,090 | 256,091 | 266,093 |
| 266,093 | 266,094 | 266,095 | 266,096 | 266,097 | 266,098 | 266,099 | 266,100 | 266,101 |
| 267,079 | 267,080 | 267,081 | 267,082 | 267,083 | 267,084 | 267,085 | 267,086 | 267,087 |
| 267,088 | 268,483 | 269,333 | 269,334 | 269,335 | 269,336 | 263,337 | 271,056 | 272,658 |
| 272,661 | 276,766 | 279,132 | 279,133 | 279,134 | 279,135 | 279,136 | 279,139 | 279,140 |
| 279,143 | 279,145 | 284,458 | 285,210 | 290,150 | 291,940 | 302,275 | 302,501 | 306,424 |
| 311,059 | 312,296 | 312,297 | 312,298 | 312,299 | 312,300 | 312,301 | 312,302 | 312,303 |
| 312,304 | 312,305 | 312,306 | 312,307 | 312,308 | 312,309 | 312,310 | 312,311 | 312,312 |
| 312,313 | 312,314 | 312,315 | 312,316 | 312,317 | 312,313 | 312,319 | 312,320 | 312,321 |
| 312,323 | 312,324 | 312,325 | 312,326 | 312,327 | 312,328 | 312,329 | 312,343 | 312,344 |
| 312,345 | 312,346 | 312,347 | 312,348 | 312,349 | 312,350 | 312,351 | 312,352 | 312,353 |
| 312,354 | 312,355 | 312,356 | 312,357 | 312,358 | 312,359 | 312,360 | 312,361 | 312,362 |
| 312,363 | 312,364 | 312,365 | 312,366 | 312,367 | 312,368 | 312,359 | 312,370 | 312,371 |
| 312,372 | 312,373 | 312,374 | 312,398 | 312,399 | 313,155 | 313,862 | 313,863 | 313,902 |
| 313,903 | 313,904 | 314,151 | 315,973 | 315,974 | 316,130 | 316,131 | 316,132 | 316,133 |
| 320,045 | 320,046 | 336,175 | 342,404 | 343,608 | 343,707 | 343,967 | 345,305 | 345,306 |
| 345,307 | 345,308 | 348,593 | 348,594 | 349,408 | 349,409 | 349,410 | 349,411 | 349,412 |
| 351,639 | 352,197 | 352,198 | 352,199 | 352,200 | 352,201 | 370,185 | 370,186 | 370,137 |
| 370,136 | 375,754 | 375,755 | 375,756 | 375,757 | 375,758 | 375,759 | 408,050 | 408,051 |
| 408,054 | 408,195 | 408,316 | 408,516 | 408,517 | 408,519 | 408,520 | 408,521 | 408,738 |
| 403,750 | 408,799 | 412,896 | 416,469 | 416,470 | 416,631 | 416,682 | 417,079 | 418,983 |
| 419,695 | 421,932 | 427,855 | 427,856 | 427,857 | 428,063 | 437,230 | 437,231 | 444,541 |
| 448,676 | 459,200 | 459,202 | 459,204 | 459,205 | 459,206 | 459,207 | 459,208 | 459,210 |
| 459,212 | 459,213 | 459,215 | 459,218 | 459,219 | 459,220 | 459,221 | 459,222 | 459,223 |
| 459,224 | 459,225 | 504,158 | 516,879 | 516,880 | 516,881 | 516,883 | 517,279 | 517,280 |
| 518,706 | 520,618 | 535,512 | 579,327 | 579,570 | 536,279 | 588,512 | 607,978 | 608,363 |
| 614,819 | 617,794 | 618,159 | 618,262 | 618,397 | 620,441 | 624,094 | 2,724,247 | 2,724,695 |
| 2,737,135 | 2,750,690 | 3,026,915 | 3,027,197 | 3,027,198 | 3,027,199 | 3,027,200 | 3,027,201 | 3,027,202 |
| 3,027,203 | 3,027,204 | 3,027,205 | 3,027,206 | 3,027,207 | 3,027,208 | 3,027,203 | 3,027,210 | 3,027,211 |
| 3,027,212 | 3,027,213 | 3,027,214 | 3,027,215 | 3,027,216 | 3,032,448 | 3,045,783 | 3,048,851 | 3,049,066 |
| 3,056,312 | 3,056,538 | 3,056,539 | 3,066,278 | 3,080,685 | 3,034,152 | 3,084,166 | 3,246,035 | 3,246,047 |
| 3,246,304 | 3,246,400 | 3,280,085 | 3,286,756 | 3,286,757 | 3,302,537 | 3,371,533 | 3,475,487 | 3,492,319 |
| 3,509,539 | 3,511,008 | 3,531,782 | 3,562,037 | 3,597,904 | 13,665,457 | 3,752,912 | 3,309,018 | 4,027,696 |
| 4,116,415 | 4,144,350 | 4,147,503 | 4,164,893 | 4,179,826 | 4,191,628 | 4,199,732 | 4,206,495 | 4,246,481 |
| 4,248,649 | 4,340,488 | 4,383,733 | 4,389,454 | 4,412,562 | 4,435,830 | 4,459,508 | 4,486,063 | 4,549,863 |
| 4,584,095 | 4,607,773 | 4,619,461 | 4,644,413 | 4,650,712 | 4,685,638 | 4,993,357 | 5,009,787 | 5,080,432 |
| 5,107,483 | 5,142,636 | 5,162,030 | 5,167,687 | 5,232,584 | 5,232,585 | 5,238,192 | 5,239,771 | 5,247,890 |
| 5,250,553 | 5,251,787 | 5,252,153 | 5,256,281 | 5,256,284 | 5,259,384 | 5,351,644 | 5,351,887 | 5,351,895 |
| 5,351,952 | 5,354,537 | 5,354,538 | 5,354,540 | 5,354,630 | 5,355,552 | 5,355,760 | 5,357,528 | 5,357,558 |
| 5,357,633 | 5,357,680 | 5,357,681 | 5,358,126 | 5,359,629 | 5,360,419 | 5,380,357 | 5,381,267 | 5,382,968 |
| 5,459,312 | 5,453,316 | 5,460,560 | 5,460,563 | 5,460,585 | 5,460,701 | 5,464,162 | 5,464,667 | 5,473,608 |
| 5,475,118 | 5,475,577 | 5,476,381 | 5,476,572 | 5,483,153 | 5,483,161 | 5,489,080 | 5,489,145 | 5,491,976 |
| 5,492,518 | 5,702,671 | 5,748,340 | 5,743,677 | 5,821,031 | 5,826,948 | 5,829,414 | 5,880,406 | 5,937,425 |
| 5,959,443 | 5,992,379 | 6,049,608 | 6,070,355 | 6,080,553 | 6,087,455 | 6,096,941 | 6,109,063 | 6,159,957 |
| 6,312,763 | 6,327,082 | 6,327,913 | 6,328,535 | 6,323,560 | 6,330,351 | 6,331,883 | 6,332,329 | 6,337,418 |
| 6,364,518 | 6,364,519 | 6,364,520 | 6,364,651 | 6,366,769 | 6,376,379 | 6,382,968 | 6,394,013 | 6,395,068 |
| 6,395,320 | 6,395,368 | 6,396,200 | 6,396,201 | 6,418,871 | 6,432,805 | 6,433,170 | 6,433,171 | 6,433,174 |
| 6,437,949 | 6,444,039 | 6,449,333 | 6,451,222 | 6,451,314 | 6,451,315 | 6,452,870 | 6,453,098 | 6,454,696 |
| 6,455,149 | 6,455,217 | 6,507,977 | 6,508,406 | 6,508,670 | 6,521,590 | 6,523,657 | 6,537,679 | 6,713,513 |
| 6,713,772 | 6,793,087 | 6,799,026 | 6,802,412 | 6,811,780 | 6,814,348 | 6,817,904 | 6,825,298 | 6,833,367 |
| 6,833,821 | 6,911,855 | 9,543,168 | 9,548,831 | 9,548,866 | 9,548,867 | 9,575,228 | 9,575,832 | 9,577,326 |
| 9,577,343 | 9,578,161 | 9,578,311 | 9,573,314 | 9,580,365 | 9,589,372 | 9,589,414 | 9,604,595 | 9,605,082 |
| 9,649,548 | 9,798,053 | 9,808,042 | 9,310,878 | 9,824,749 | 9,837,036 | 9,860,187 | 9,860,551 | 9,862,801 |
| 9,865,965 | 9,888,902 | 9,931,103 | 9,936,090 | 9,949,579 | 9,949,580 | 9,954,856 | 10,023,468 | 10,024,149 |
| 10,069,126 | 10,090,976 | 10,257,888 | 10,312,004 | 10,338,262 | 10,345,828 | 10,349,701 | 10,386,772 | 10,394,431 |
| 10,431,022 | 10,443,824 | 10,477,524 | 10,505,812 | 10,508,513 | 10,530,076 | 10,577,925 | 10,643,908 | 10,649,763 |
| 10,650,069 | 10,651,033 | 10,666,444 | 10,671,140 | 10,674,237 | 10,697,032 | 10,744,586 | 10,745,819 | 10,746,567 |
| 10,748,489 | 10,757,607 | 10,816,826 | 10,834,120 | 10,840,702 | 10,876,483 | 10,878,406 | 10,878,407 | 10,936,360 |
| 10,981,618 | 11,002,054 | 11,005,114 | 11,049,085 | 11,063,982 | 11,077,325 | 11,103,145 | 11,187,997 | 11,195,304 |
| 11,211,437 | 11,224,375 | 11,232,487' | 11,251,075 | 11,269,492 | 11,311,393 | 11,334,638 | 11,336,975 | 11,369,488 |
| 11,371,427 | 11,403,966 | 11,435,113 | 11,452,073 | 11,468,263 | 11,559,479 | 11,560,431 | 11,624,317 | 11,738,836 |
| 11,750,101 | 11,800,790 | 11,801,096 | 11,813,954 | 11,813,954 | 11,949,825 | 11,949,826 | 11,949,827 | 11,949,992 |
| 11,949,993 | 11,949,994 | 11,949,995 | 11,949,996 | 11,949,997 | 11,950,160 | 11,952,475 | 11,952,476 | 11,952,477 |
| 11,971,649 | 11,971,650 | 11,980,558 | 11,985,990 | 11,986,029 | 11,987,433 | 12,049,081 | 12,049,082 | 12,049,083 |
| 12,112,343 | 12,305,258 | 12,305,260 | 12,485,864 | 12,485,867 | 12,485,868 | 12,485,869 | 12,485,370 | 12,595,784 |
| 12,645,905 | 12,654,670 | 12,736,159 | 12,736,220 | 12,747/453 | 12,756,830 | 12,795,887 | 12,824,390 | 12,832,980 |
| 12,832/981 | 12,884,523 | 13,047,346 | 13,050,612 | 13,134,261 | 13,212,303 | 13,212,306 | 13,212,312 | 13,302,718 |
| 13,302,719 | 13,416,700 | 13,443,227 | 13,443,228 | 13,516,476 | 13,613,089 | 13,643,880 | 13,678,819 | 13,678,824 |
| 13,678,825 | 13,683,693 | 13,726,122 | 13,734,063 | 13,743,985 | 13,750,255 | 13,765,615 | 13,765,616 | 13,305,634 |
| 13,805,693 | 13,812,873 | 13,813,546 | 13,829,286 | 13,909,037 | 13,955,960 | 13,980,772 | 13,981,841 | 14,009,635 |
| 14,066,966 | 14,066,967 | 14,094,413 | 14,122,911 | 14,178,438 | 14,205,366 | 14,205,726 | 14,205,728 | 14,205,730 |
| 14,205,731 | 14,223,279 | 14,389,153 | 14,401,159 | 14,401,163 | '14,453,762 | 14,513,380 | 14,671,601 | 14,700,083 |
| 14,744,953 | 14,734,649 | 14,818,755 | 14,833,399 | 14,876,330 | 14,376,967 | 14,902,319 | 14,912,293 | 14,921,144 |
| 15,001,088 | 15,012,428 | 15,072,186 | 15,211,288 | 15,225,244 | 15,225,439 | 15,360,813 | 15,362,043 | 15,394,753 |
| 15,412,253 | 15,412,285 | 15,412,290 | 15,535,760 | 15,536,995 | 15,638,459 | 15,787,841 | 15,796,321 | 16,211,134 |
| 16,211,720 | 16,213,153 | 16,682,839 | 16,683,367 | 16,684,317 | 16,635,520 | 16,635,995 | 16,696,532 | 16,697,858 |
| 16,697,859 | 16,697,860 | 16,697,861 | 16,697,362 | 16,697,863 | 16,703,059 | 16,704,216 | 16,704,429 | 16,704,431 |
| 16,705,057 | 16,714,200 | 16,714,201 | 17,753,879 | 17,753,924 | 17,972,451 | 13,006,662 | 18,362,488 | 18,401,442 |
| 18,426,515 | 18,442,047 | 18,458,521 | 18,471,244 | 18,513,358 | 18,513,372 | 18,544,746 | 18,620,404 | 18,620,407 |
| 18,633,056 | 18,633,053 | 18,678,997 | 13,690,655 | 13,699,569 | 18,760,606 | 18,760,618 | 18,760,569 | 18,762,179 |
| 18,764,458 | 18,735,193 | 18,735,194 | 13,798,734 | 13,942,540 | 18,942,541 | 18,944,705 | 18,950,086 | 18,956,772 |
| 18,981,320 | 18,982,476 | 18,982,574 | 19,023,078 | 19,026,428 | 19,026,430 | 19,026,432 | 19,026,436 | 19,026,437 |
| 19,026,438 | 19,026,441 | 19,032,110 | 19,044,210 | 19,067,415 | 19,077,033 | 19,105,252 | 19,347,785 | 19,350,187 |
| 19,360,272 | 19,382,081 | 19,609,626 | 19,609,639 | 19,702,616 | 19,739,449 | 19,739,453 | 19,739,455 | 19,748,686 |
| 19,751,144 | 19,757,136 | 19,757,143 | 19,757,145 | 19,757,146 | 19,762,759 | 19,786,074 | 19,790,659 | 19,795,274 |
| 19,827,519 | 19,833,769 | 19,836,514 | 19,872,105 | 19,882,894 | 19,893,232 | 19,898,354 | 19,968,620 | 19,981,220 |
| 20,031,692 | 20,035,298 | 20,035,299 | 20,038,204 | 20,042,688 | 20,054,866 | 20,054,981 | 20,055,037 | 20,055,252 |
| 20,056,599 | 20,056,600 | 20,056,785 | 20,063,715 | 20,063,742 | 20,063,039 | 20,083,766 | 20,083,785 | 20,083,796 |
| 20,083,797 | 20,085,186 | 20,095,232 | 20,143,803 | 20,144,054 | 20,152,548 | 20,152,549 | 20,152,701 | 20,154,552 |
| 20,202,607 | 20,202,656 | 20,207,034 | 20,209,276 | 20,209,388 | 20,211,642 | 20,246,464 | 20,271,624 | 20,287,272 |
| 20,288,961 | 20,360,926 | 20,371,762 | 20,445,407 | 20,474,394 | 20,474,403 | 20,474,405 | 20,480,138 | 20,482,994 |
| 20,484,753 | 20,486,182 | 20,488,746 | 20,503,507 | 20,503,509 | 120,503,51 | 20,503,512 | 20,503,514 | 20,512,536 |
| 20,516,635 | 20,529,308 | 20,531,312 | 20,542,969 | 20,549,509 | 20,558,446 | 20,569,120 | 20,634,001 | 20,693,889 |
| 20,832,953 | 20,836,092 | 20,837,621 | 20,838,029 | 20,838,030 | 20,838,042 | 20,841,764 | 20,841,765 | 20,844,702 |
| 20,844,703 | 20,845,094 | 20,845,106 | 20,849,356 | 20,975,696 | 20,977,035 | 20,977,049 | 20,978,299 | 21,114,753 |
| 21,114,754 | 21,114,763 | 21,114,764 | 21,114,783 | 21,114,860 | 21,115,629 | 21,117,047 | 21,119,391 | 21,120,402 |
| 21,120,735 | 21,120,889 | 21,120,928 | 21,121,096 | 21,121,135 | 21,122,097 | 21,125,668 | 21,126,516 | 21,127,315 |
| 21,127,316 | 21,127,707 | 21,139,502 | 21,139,503 | 21,139,534 | 21,139,535 | 21,141,030 | 21,141,081 | 21,152,149 |
| 21,154,358 | 21,183,466 | 21,225,639 | 21,225,707 | 21,252,377 | 21,252,378 | 21,321,841 | 21,391,529 | 21,453,257 |
| 21,455,200 | 21,459,652 | 21,491,032 | 21,491,033 | 21,528,715 | 21,536,734 | 21,536,760 | 21,536,766 | 21,536,770 |
| 21,536,773 | 21,536,775 | 21,536,777 | 21,536,779 | 21,536,783 | 21,544,015 | 21,584,652 | 21,584,653 | 21,612,055 |
| 21,624,658 | 21,646,552 | 21,715,449 | 21,715,463 | 21,716,090 | 21,732,898 | 21,748,563 | 21,752,591 | 21,760,500 |
| 21,760,511 | 21,798,783 | 21,808,506 | 21,845,204 | 21,880,523 | 21,381,274 | 21,881,315 | 21,881,945 | 21,888,690 |
| 21,888,691 | 21,910,092 | 21,910,161 | 21,910,167 | 21,910,205 | 21,910,220 | 21,910,731 | 21,924,641 | 21,930,582 |
| 21,944,883 | 21,960,621 | 21,971,281 | 21,998,298 | 21,998,316 | 22,023,671 | 22,044,206 | 22,066,493 | 22,138,168 |
| 22,138,169 | 22,141,747 | 22,184,136 | 22,234,755 | 22,237,127 | 22,237,128 | 22,238,516 | 22,256,074 | 22,323,793 |
| 22,336,054 | 22,342,054 | 22,342,102 | 22,342,120 | 22,342,127 | 22,342,155 | 22,342,157 | 22,342,208 | 22,342,232 |
| 22,342,254 | 22,342,255 | 22,342,257 | 22,342,266 | 22,342,270 | 22,439,115 | 22,450,932 | 22,479,650 | 22,481,576 |
| 22,495,054 | 22,572,164 | 22,596,865 | 22,603,871 | 22,635,575 | 22,693,028 | 22,693,029 | 22,708,915 | 22,718,963 |
| 22,743,281 | 22,743,405 | 22,795,593 | 22,804,163 | 22,874,163 | 22,898,390 | 22,988,986 | 22,988,999 | 22,989,035 |
| 23,019,778 | 23,034,996 | 23,054,262 | 23,079,060 | 23,132,927 | 23,132,935 | 23,132,951 | 23,132,964 | 23,132,971 |
| 23,132,989 | 23,133,025 | 23,133,075 | 23,133,081 | 23,133,107 | 23,133,123 | 23,133,145 | 23,133,154 | 23,133,219 |
| 23,175,350 | 23,182,090 | 23,194,872 | 23,223,696 | 23,234,551 | 23,234,552 | 23,234,553 | 23,236,023 | 23,236,101 |
| 23,236,101 | 23,237,635 | 23,237,636 | 23,237,637 | 23,265,870 | 23,265,878 | 23,265,893 | 23,265,971 | 23,266,003 |
| 23,266,004 | 23,267,272 | 23,267,743 | 23,268,153 | 23,268,251 | 23,268,362 | 23,268,493 | 23,268,536 | 23,268,879 |
| 23,268,994 | 23,269,122 | 23,269,200 | 23,269,208 | 23,269,478 | 23,269,479 | 23,269,535 | 23,293,286 | 23,293,287 |
| 23,293,288 | 23,297,479 | 23,317,906 | 23,339,742 | 23,371,233 | 23,414,025 | 23,422,913 | 23,422,919 | 23,422,971 |
| 23,423,076 | 23,423,302 | 23,423,303 | 23,423,304 | 23,423,305 | 23,423,306 | 23,423,307 | 23,423,308 | 23,423,309 |
| 23,423,310 | 23,423,311 | 23,423,384 | 23,423,385 | 23,462,625 | 23,509,052 | 23,615,241 | 23,615,242 | 23,615,614 |
| 23,615,615 | 23,615,616 | 23,616,635 | 23,617,006 | 23,617,095 | 23,617,096 | 23,617,097 | 23,617,098 | 23,617,099 |
| 23,617,100 | 23,617,156 | 23,617,157 | 23,617,770 | 23,618,480 | 23,618,483 | 23,618,434 | 23,618,493 | 23,618,494 |
| 23,618,537 | 23,618,592 | 23,618,632 | 23,618,633 | 23,618,636 | 23,618,697 | 23,618,805 | 23,618,840 | 23,618,850 |
| 23,618,351 | 23,618,852 | 23,618,853 | 23,618,854 | 23,618,910 | 23,613,953 | 23,619,068 | 23,619,958 | 23,621,942 |
| 23,621,970 | 23,622,126 | 23,622,688 | 23,622,689 | 23,623,716 | 23,623,741 | 23,623,742 | 23,639,731 | 23,644,513 |
| 23,664,719 | 23,665,572 | 23,665,811 | 23,667,268 | 23,667,269 | 23,668,632 | 23,670,523 | 23,670,848 | 23,671,187 |
| 23,673,647 | 23,673,838 | 23,675,353 | 23,675,510 | 23,675,762 | 23,676,536 | 23,677,060 | 23,678,030 | 23,678,031 |
| 23,678,498 | 23,678,861 | 23,679,054 | 23,679,059 | 23,679,327 | 23,631,930 | 23,634,591 | 23,684,831 | 23,684,835 |
| 23,687,156 | 23,688,636 | 23,689,040 | 23,690,436 | 23,692,071 | 23,695,993 | 23,699,679 | 23,701,920 | 23,702,397 |
| 23,704,689 | 23,704,946 | 23,705,691 | 23,711,769 | 23,712,046 | 23,712,047 | 23,714,612 | 23,714,613 | 23,714,614 |
| 23,714,615 | 23,714,616 | 23,719,509 | 23,719,536 | 23,725,018 | 24,180,936 | 24,181,706 | 24,181,707 | 24,181,708 |
| 24,181,711 | 24,131,835 | 24,131,841 | 24,181,927 | 24,182,103 | 24,182,110 | 24,182,111 | 24,182,114 | 24,182,116 |
| 24,182,325 | 24,132,334 | 24,132,367 | 24,183,873 | 24,183,928 | 24,184,262 | 24,184,949 | 24,185,838 | 24,188,024 |
| 24,188,025 | 24,190,450 | 24,192,474 | 24,192,485 | 24,196,464 | 24,196,465 | 24,198,998 | 24,319,283 | 24,333,845 |
| 24,833,846 | 24,833,847 | 24,833,848 | 24,833,849 | 24,833,850 | 24,833,851 | 24,833,852 | 24,833,853 | 24,833,854 |
| 24,833,855 | 24,333,856 | 24,833,857 | 24,833,858 | 24,833,359 | 24,833,860 | 24,833,861 | 24,833,862 | 24,833,863 |
| 24,833,864 | 24,333,865 | 24,833,866 | 24,833,867 | 24,833,368 | 24,833,869 | 24,833,870 | 24,833,871 | 24,833,872 |
| 24,833,873 | 24,833,874 | 24,833,875 | 24,833,876 | 24,333,877 | 24,833,878 | 24,833,884 | 24,834,489 | 24,834,490 |
| 24,834,491 | 24,834,492 | 24,834,493 | 24,834,494 | 24,834,495 | 24,334,496 | 24,834,497 | 24,834,498 | 24,834,499 |
| 24,836,124 | 24,838,478 | 24,838,850 | 24,838,851 | 24,338,852 | 24,340,406 | 24,847,717 | 24,847,718 | 24,884,165 |
| 25,021,277 | 25,021,716 | 25,022,094 | 25,022,139 | 25,059,847 | 25,147,438 | 25,201,247 | 25,202,100 | 25,215,427 |
| 25,258,926 | 42,616,376 | 42,626,516 | 42,626,649 | 42,626,869 | 43,834,996 | 44,134,551 | 44,134,552 | 44,135,769 |
| 44,135,770 | 44,135,830 | 44,135,912 | 44,144,455 | 44,146,053 | 44,146,486 | 44,146,567 | 44,146,730 | 44,146,916 |
| 44,147,059 | 44,148,502 | 44,150,404 | 44,150,532 | 44,150,701 | 44,150,943 | 44,151,798 | 44,152,237 | 44,152,840 |
| 44,154,459 | 44,154,433 | 44,154,512 | 44,154,561 | 44,154,582 | 44,154,630 | 44,154,866 | 44,154,920 | 44,321,327 |
| 44,369,986 | 44,370,063 | 44,558,881 | 44,558,882 | 44,593,649 | 44,603,086 | 44,630,230 | 44,720,830 | 44,721,082 |
| 44,721,083 | 44,725,754 | 45,048,798 | 45,048,892 | 45,050,564 | 45,051,682 | 45,266,914 | 45,479,758 | 46,224,551 |
| 46,224,552 | 46,224,593 | 46,875,030 | 46,936,754 | 49,789,061 | 50,396,934 | 50,910,470 | 50,920,338 | 50,921,205 |
| 50,921,206 | 50,930,983 | 50,931,253 | 50,931,674 | 50,932,276 | 50,933,749 | 50,989,345 | 50,989,963 | 51,000,486 |
| 53,339,029 | 53,384,454 | 53,393,493 | 53,393,598\| | 53,400,626 | 53,405,325 | 53,428,512 | 53,439,260 | 53,462,054 |
| 53,463,799 | 53,471,871 | 53,471,920 | 54,593,359 | 54,599,412 | 54,601,637 | 54,602,507 | 54,602,768 | 54,602,830 |
| 54,603,132 | 54,603,241 | 54,603,485 | 54,603,489 | 54,603,573 | 54,603,865 | 54,604,418 | 54,605,078 | 54,605,477 |
| 54,605,894 | 54,606,603 | 54,607,273 | 54,610,700 | 54,611,643 | 54,612,078 | 54,612,079 | 56,841,614 | 56,841,734 |
| 56,842,095 | 56,842,269 | 56,843,347 | 56,843,418 | 56,843,823 | 56,925,092 | 56,955,921 | 56,955,922 | 56,955,923 |
| 56,955,924 | 57,347,038 | 57,347,038 | 57,347,159 | 57,347,436 | 57,343,041 | 57,343,043 | 57,348,098 | 57,348,555 |
| 57,348,941 | 57,349,419 | 57,349,472 | 57,349,580 | 57,349,581 | 57,350,047 | 57,350,047 | 57,350,641 | 57,350,785 |
| 57,350,803 | 57,351,175 | 57,351,442 | 57,351,614 | 57,351,668 | 57,351,752 | 57,352,657 | 57,353,494 | 57,358,064 |
| 57,358,206 | 57,358,236 | 57,359,122 | 57,359,123 | 57,365,615 | 57,336,366 | 57,397,090 | 57,418,239 | 57,422,000 |
| 57,422,093 | 57,448,801 | 57,448,802 | 57,443,399 | 57,449,070 | 57,449,169 | 57,449,394 | 57,450,418 | 57,451,424 |
| 57,459,958 | 57,464,763 | 57,464,821 | 57,464,908 | 57,464,921 | 57,465,286 | 57,466,691 | 57,466,703 | 57,469,302 |
| 57,469,420 | 57,485,235 | 57,485,401 | 57,490,558 | 57,507,417 | 57,507,848 | 57,507,876 | 57,514,279 | 57,514,722 |
| 57,517,132 | 57,517,138 | 57,517,933 | 57,763,375 | 58,165,711 | 58,165,712 | 58,165,713 | 58,165,714 | 58,165,715 |
| 58,165,716 | 58,165,717 | 58,165,718 | 58,165,720 | 58,165,721 | 58,165,723 | 58,165,724 | 58,165,726 | 58,165,727 |
| 58,165,730 | 58,165,731 | 58,165,733 | 58,165,734 | 58,165,735 | 58,165,736 | 58,165,737 | 58,165,738 | 58,302,438 |
| 58,671,705 | 58,671,705 | 58,729,368 | 58,729,369 | 58,729,370 | 58,729,371 | 58,729,372 | 58,729,373 | 58,729,374 |
| 53,729,375 | 58,729,377 | 58,729,379 | 58,729,380 | 58,729,381 | 58,729,382 | 58,729,383 | 53,729,384 | 58,729,387 |
| 53,729,388 | 58,729,389 | 58,729,390 | 58,729,391 | 58,729,392 | 58,729,393 | 58,729,394 | 53,729,397 | 58,729,398 |
| 58,729,399 | 58,886,963 | 59,263,990 | 59,467,531 | 59,467,532 | 59,467,533 | 59,467,534 | 59,467,536 | 59,467,537 |
| 59,467,538 | 59,467,539 | 59,467,540 | 59,467,542 | 59,467,543 | 59,467/545 | 59,467,546 | 59,467,549 | 59,467,550 |
| 59,467,552 | 59,467,553 | 59,467,554 | 59,467,556 | 59,467,557 | 59,865,558 | 59,940,674 | 59,940,675 | 60,036,552 |
| 60,036,553 | 60,165,464 | 60,208,620 | 60,208,624 | 60,208,652 | 66,545,852 | 70,297,382 | 70,675,072 | 70,675,073 |
| 70,700,060 | 70,702,302 | 71,299,673 | 71,300,916 | 71,308,157 | 71,313,437 | 71,313,488 | 71,317,296 | 71,319,306 |
| 7I,319,307 | 71,319,865 | 71,327,504 | 71,328,225 | 71,330,499 | 71,331,141 | 71,332,057 | 7l,334,109 | 71,335,003 |
| 71,335,466 | 71,335,471 | 71,336,056 | 71,337,069 | 71,338,314 | 71,339,210 | 71,339,611 | 71,339,840 | 71,340,597 |
| 71,342,472 | 71,348,363 | 71,348,670 | 71,354,187 | 71,357,095 | 71,359,057 | 71,360,254 | 71,361,127 | 71,361,431 |
| 71,363,441 | 71,363,442 | 71,363,446 | 71,363,802 | 71,364,348 | 71,364,378 | 71,365,365 | 71,366,353 | 71,367,154 |
| 71,367,770 | 71,367,894 | 71,367,895 | 71,368,197 | 71,368,508 | 71,370,280 | 71,371,122 | 71,371,820 | 71,372,565 |
| 71,372,674 | 71,380,037 | 71,380,573 | 71,381,041 | 71,381,042 | 71,382,184 | 71,332,439 | 71,382,445 | 71,385,726 |
| 71,385,727 | 71,387,140 | 71,387,143 | 71,387,509 | 71,394,297 | 71,394,403 | 71,394,404 | 71,397,369 | 71,398,232 |
| 71,399,333 | 73,401,268 | 71,401,326 | 71,401,532 | 71,401,921 | 71,405,075 | 71,408,659 | 71,409,840 | 71,410,005 |
| 71,410,222 | 71,410,223 | 71,421,391 | 71,430,951 | 71,430,977 | 71,431,024 | 71,431,066 | 71,436,586 | 71,437,827 |
| 71,444,769 | 71,444,988 | 71,446,559 | 71,446,674 | 71,449,004 | 71,459,631 | 71,479,335 | 71,487,561 | 71,586,823 |
| 71,586,365 | 71,586,922 | 71,710,894 | 71,774,626 | 72,941,434 | 73,415,795 | 73,427,316 | 73,427,319 | 73,427,321 |
| 73,555,192 | 73,555,265 | 73,556,100 | 73,894,235 | 73,894,259 | 73,894,260 | 73,894,261 | 73,894,262 | 73,894,263 |
| 73,894,264 | 73,894,265 | 73,894,266 | 73,894,267 | 73,894,268 | 73,894,269 | 73,894,270 | 73,894,271 | 73,894,272 |
| 73,894,273 | 73,394,274 | 73,894,275 | 73,894,276 | 73,894,277 | 73,894,273 | 73,897,259 | 73,951,871 | 73,994,974 |
| 74,764,745 | 74,765,628 | 74,765,632 | 74,765,637 | 75,124,214 | 76,198,490 | 76,419,686 | 76,957,520 | 76,958,471 |
| 73,063,565 | 78,064,930 | 78,066,137 | 78,067,178 | 78,067,215 | 78,067,216 | 82,030,612 | 82,030,683 | 82,316,598 |
| 82,316,626 | 82,316,722 | 82,318,949 | 82,363,720 | 82,363,766 | 83,768,323 | 83,773,659 | 84,226,900 | 84,228,930 |
| 84,228,952 | 84,229,512 | 84,229,564 | 84,230,181 | 84,230,317 | 84,230,491 | 84,230,496 | 84,230,505 | 84,232,747 |
| 84,251,193 | 84,271,289 | 84,271,519 | 84,271,720 | 84,273,649 | 84,293,452 | 84,313,624 | 84,317,251 | 84,317,410 |
| 84,317,582 | 84,317,592 | 84,317,617 | 84,317,748 | 84,317,859 | 84,317,938 | 84,318,269 | 84,318,352 | 84,318,548 |
| 84,318,697 | 84,319,320 | 84,319,537 | 84,313,696 | 84,319,949 | 84,320,164 | 84,320,412 | 84,320,471 | 84,321,900 |
| 84,321,961 | 84,322,271 | 84,322,555 | 84,322,803 | 84,323,164 | 84,323,196 | 84,323,348 | 84,323,444 | 84,331,163 |
| 84,331,138 | 85,671,405 | 85,677,163 | 85,683,074 | 85,764,410 | 85,844,805 | 85,858,957 | 85,901,868 | 85,970,572 |
| 86,171,938 | 86,181,093 | 86,278,207 | 86,341,919 | 86,341,922 | 86,618,681 | 86,618,633 | 86,618,685 | 86,743,524 |
| 86,743,525 | 86,745,607 | 87,058,047 | 87,059,370 | 87,072,965 | 87,073,127 | 87,073,128 | 87,073,129 | 87,073,143 |
| 87,073,508 | 87,036,209 | 87,036,388 | 87,090,261 | 87,106,887 | 87,110,451 | 87,131,993 | 87,131,994 | 87,131,995 |
| 87,131,996 | 87,131,997 | 87,132,131 | 87,132,524 | 87,132,525 | 87,132,526 | 87,144,976 | 87,165,315 | 87,172,195 |
| 87,172,196 | 87,195,630 | 87,205,220 | 87,205,221 | 87,205,976 | 87,233,402 | 87,233,403 | 87,234,663 | 87,234,712 |
| 87,238,627 | 87,238,628 | 87,238,633 | 87,240,211 | 87,251,088 | 87,255,861 | 87,255,862 | 87,255,870 | 87,255,875 |
| 87,255,880 | 87,255,881 | 87,269,470 | 87,263,477 | 87,314,093 | 87,318,793 | 87,330,514 | 87,344,746 | 87,350,630 |
| 87,359,256 | 87,438,458 | 87,433,459 | 87,433,573 | 87,438,574 | 87,438,777 | 87,438,779 | 87,458,202 | 87,458,203 |
| 87,458,204 | 87,458,727 | 87,472,400 | 87,472,523 | 87,472,811 | 87,472,864 | 87,473,748 | 87,474,375 | 87,474,815 |
| 87,475,704 | 87,476,010 | 87,476,169 | 87,476,500 | 87,476,559 | 87,476,847 | 87,477,538 | 87,477,549 | 87,478,368 |
| 87,491,392 | 87,491,700 | 87,492,961 | 87,499,136 | 87,501,741 | 87,505,017 | 87,510,533 | 87,514,724 | 87,514,827 |
| 87,562,629 | 87,603,632 | 87,606,799 | 87,606,893 | 87,606,894 | 87,606,895 | 87,640,134 | 87,656,152 | 87,657,080 |
| 87,657,747 | 87,662,947 | 87,671,297 | 87,671,298 | 87,671,912 | 87,699,275 | 87,699,545 | 87,711,479 | 87,721,746 |
| 87,735,226 | 87,733,731 | 87,733,732 | 87,739,172 | 87,743,549 | 87,755,255 | 87,756,357 | 87,756,359 | 87,762,269 |
| 87,763,110 | 87,764,999 | 87,765,000 | 87,770,098 | 87,770,571 | 87,805,032 | 87,805,033 | 87,841,306 | 87,341,308 |
| 87,852,890 | 87,934,277 | 87,935,205 | 87,945,130 | 87,957,607 | 87,979,929 | 88,005,023 | 88,006,712 | 88,027,044 |
| 88,027,224 | 88,027,225 | 88,027,247 | 88,027,249 | 88,027,349 | 88,027,898 | 88,028,174 | 88,028,175 | 88,028,382 |
| 88,047,523 | 88,048,162 | 88,048,239 | 88,048,240 | 88,048,687 | 88,048,639 | 88,048,692 | 88,048,693 | 88,085,429 |
| 88,085,430 | 88,035,672 | 88,035,673 | 88,085,737 | 88,085,790 | 88,085,832 | 88,086,427 | 88,086,990 | 88,090,309 |
| 88,103,022 | 88,112,731 | 88,112,732 | 88,113,921 | 88,162,367 | 88,162,370 | 88,163,324 | 88,171,189 | 88,195,082 |
| 88,195,499 | 88,199,335 | 88,202,050 | 88,223,011 | 88,234,680 | 88,237,233 | 88,247,261 | 88,247,263 | 88,282,576 |
| 88,282,578 | 88,288,218 | 88,312,532 | 88,314,934 | 88,314,935 | 88,327,893 | 88,328,145 | 83,339,646 | 88,339,769 |
| 88,339,770 | 88,342,230 | 88,360,632 | 88,367,464 | 88,357,467 | 88,370,013 | 88,371,460 | 88,371,540 | 88,383,641 |
| 88,384,192 | 88,402,848 | 88,404,033 | 88,414,089 | 88,421,853 | 88,421,854 | 88,423,689 | 88,423,693 | 88,423,694 |
| 88,423,747 | 88,424,044 | 88,424,045 | 88,424,429 | 88,440,204 | 88,440,205 | 88,446,062 | 88,453,441 | 88,454,696 |
| 88,458,282 | 88,463,436 | 88,470,125 | 88,470,129 | 88,473,659 | 88,473,660 | 88,481,422 | 88,488,676 | 88,510,887 |
| 88,602,316 | 88,610,141 | 88,610,142 | 83,611,072 | 88,613,537 | 88,613,538 | 88,613,539 | 88,633,323 | 88,633,324 |
| 88,636,491 | 88,640,649 | 88,641,981 | 88,641,982 | 88,642,858 | 88,642,860 | 88,670,623 | 88,680,217 | 88,680,218 |
| 88,681,635 | 88,681,637 | 88,683,805 | 88,688,468 | 88,709,342 | 88,714,945 | 88,717,455 | 88,728,174 | 88,728,830 |
| 88,736,668 | 88,788,483 | 88,740,953 | 88,741,022 | 88,741,024 | 88,745,619 | 88,746,538 | 88,756,177 | 88,762,995 |
| 88,763,319 | 88,786,396 | 88,791,224 | 88,807,784 | 88,807,786 | 88,827,124 | 88,828,033 | 88,836,636 | 88,347,453 |
| 88,848,338 | 89,423,442 | 89,573,826 | 89,972,012 | 89,977,504 | 90,021,411 | 90,028,968 | 90,471,423 | 90,472,865 |
| 90,472,953 | 90,472,954 | 90,476,144 | 90,476,459 | 90,478,957 | 90,479,639 | 90,657,387 | 90,657,388 | 90,661,369 |
| 90,661,495 | 91,617,718 | 91,618,135 | 91,809,010 | 91,809,010 | 91,372,575 | 91,872,576 | 91,873,289 | 91,885,296 |
| 91,886,247 | 91,836,305 | 91,836,306 | 91,386,379 | 91,977,524 | 91,977,525 | 91,979,699 | 91,979,700 | 91,979,701 |
| 91,979,702 | 91,982,311 | 91,990,907 | 91,990,908 | 91,996,625 | 91,997,872 | 91,997,873 | 91,998,119 | 91,998,120 |
| 91,998,157 | 91,998,324 | 92,003,746 | 92,003,947 | 92,004,943 | 92,006,598 | 92,006, | 92,006,915 | 92,006,992 |
| 92,006,993 | 92,006,994 | 92,006,995 | 92,006,996 | 92,006,997 | 92,006,998 | 92,006,999 | 92,007,000 | 92,007,001 |
| 92,007,002 | 92,007,003 | 92,007,004 | 92,007,005 | 92,007,006 | 92,007,007 | 92,007,008 | 92,007,091 | 92,007,010 |
| 92,007,011 | 92,007,017 | 92,007,451 | 92,008,067 | 92,008,243 | 92,008,621 | 92,009,736 | 92,010,994 | 92,010,995 |
| 92,010,996 | 92,010,997 | 92,013,326 | 92,022,904 | 92,024,946 | 92,024,947 | 92,025,093 | 92,025,094 | 92.025,095 |
| 92,025,096 | 92,025,097 | 92,025,098 | 92,025,099 | 92,025,100 | 92,025,114 | 92,025,115 | 92,025,116 | 92,025,117 |
| 92,025,118 | 92,025,119 | 92,025,120 | 92,025,121 | 92,025,124 | 92,026,484 | 92,026,485 | 92,026,502 | 92,026,503 |
| 92,026,560 | 92,026,596 | 92,026,616 | 92,027,384 | 92,027,405 | 92,027,424 | 92,027,436 | 92,028,137 | 92,028,138 |
| 92,028,742 | 92,028,792 | 92,028,793 | 92,028,841 | 92,028,912 | 92,038,599 | 92,042,585 | 92,043,501 | 92,043,987 |
| 92,131,721 | 92,135,763 | 100,919,003 | 100,931,586 | 100,934,184 | 100,952,630 | 100,963,338 | 100,963,929 | 100,986,762 |
| 100,986,764 | 100,993,010 | 100,999,029 | 101,005,691 | 101,006,949 | 101,009,191 | 101,009,416 | 101,015,656 | 101,025,050 |
| 101,025,051 | 101,046,544 | 101,052,365 | 101,053,279 | 101,053,279 | 101,053,279 | 101,053,280 | 101,064,349 | 101,071,247 |
| 101,093,527 | 101,100,195 | 101,100,195 | 101,116,060 | 101,118,643 | 101,118,644 | 101,122,537 | 101,127,381 | 101,136,872 |
| 101,137,899 | 101,139,985 | 101,139,936 | 101,200,916 | 101,211,580 | 101,230,437 | 101,230,438 | 101,230,439 | 101,243,708 |
| 101,255,615 | 101,257,613 | 101,261,664 | 101,288,890 | 101,298,727 | 101,313,322 | 101,340,809 | 101,348,281 | 101,354,488 |
| 101,354,489 | 101,357,168 | 101,357,169 | 101,357,594 | 101,357,595 | 101,377,149 | 101,397,671 | 101,409,658 | 101,415,375 |
| 101,415,376 | 101,440,809 | 101,458,324 | 101,458,325 | 101,458,327 | 101,478,419 | 101,495,974 | 101,504,033 | 101,522,049 |
| 101,533,057 | 101,564,671 | 101,586,483 | 101,599,593 | 101,599,594 | 101,611,448 | 101,614,891 | 101,616,131 | 101,625,879 |
| 101,625,830 | 101,625,331 | 101,625,332 | 101,635,676 | 101,652,314 | 101,652,556 | 101,652,557 | 101,652,558 | 101,652,559 |
| 101,664,275 | 101,664,531 | 101,664,931 | 101,667,977 | 101,674,978 | 101,674,979 | 101,674,980 | 101,674,981 | 101,674,982 |
| 101,674,983 | 101,686,450 | 101,689,360 | 101,691,534 | 101,691,535 | 101,691,536 | 101,691,537 | 101,691,538 | 101,691,539 |
| 101,691,632 | 101,691,633 | 101,691,634 | 101,691,635 | 101,691,636 | 101,691,637 | 101,699,244 | 101,708,462 | 101,708,463 |
| 101,715,578 | 101,716,440 | 101,723,246 | 101,786,483 | 101,786,862 | 101,786,863 | 101,805,070 | 101,846,346 | 101,847,764 |
| 101,883,650 | 101,889,836 | 101,889,839 | 101,895,131 | 101,895,132 | 101,913,583 | 101,916,334 | 101,928,003 | 101,929,420 |
| 101,930,742 | 101,946,400 | 101,970,427 | 101,972,020 | 101,978,331 | 101,978,342 | 101,978,385 | 101,988,596 | 101,993,105 |
| 101,996,063 | 102,012,028 | 102,013,216 | 102,013,636 | 102,039,635 | 102,054,416 | 102,072,502 | 102,075,650 | 102,129,552 |
| 102,137,137 | 102,160,647 | 102,171,729 | 102,215,006 | 102,239,767 | 102,248,352 | 102,261,461 | 102,279,703 | 102,279,704 |
| 102,279,705 | 102,279,706 | 102,279,707 | 102,279,708 | 102,279,709 | 102,283,040 | 102,283,045 | 102,283,046 | 102,326,471 |
| 102,393,511 | 102,409,673 | 102,413,161 | 102,416,167 | 102,438,080 | 102,439,215 | 102,533,224 | 102,595,004 | 102,595,005 |
| 102,601,898 | 110,210,780 | 110,210,731 | 110,431,250 | 110,499,257 | 110,499,461 | 110,499,630 | 110,499,730 | 110,499,847 |
| 110,500,056 | 110,500,265 | 110,500,474 | 110,500,682 | 110,500,891 | 110,501,100 | 110,501,309 | 110,501,518 | 110,501,727 |
| 110,501,936 | 110,502,145 | 110,502,354 | 112,501,307 | 112,501,308 | 112,501,309 | 112,501,310 | 112,501,311 | 112,501,312 |
| 112,501,313 | 112,501,314 | 112,501,315 | 112,501,316 | 112,501,317 | 112,501,318 | 112,501,319 | 112,501,320 | 112,501,321 |
| 112,501,322 | 112,501,323 | 112,501,324 | 112,501,325 | 112,501,326 | 112,501,327 | 112,501,328 | 112,501,329 | 112,501,330 |
| 112,501,331 | 112,501,332 | 112,501,333 | 112,501,334 | 112,501,335 | 112,501,336 | 112,501,337 | 112,501,338 | 112,501,339 |
| 117,059,131 | 118,984,370 | 118,985,489 | 118,985,493 | 121,233,249 | 121,233,660 | 121,233,703 | 122,201,230 | 122,228,415 |
| 122,363,780 | 122,363,783 | 122,403,017 | 123,394,049 | 124,204,125 | 129,318,123 | 129,318,137 | 129,627,693 | 129,629,450 |
| 129,629,459 | 129,629,462 | 129,629,468 | 129,629,516 | 129,629,522 | 129,629,530 | 129,630,137 | 129,631,095 | 129,632,050 |
| 129,632,463 | 129,632,826 | 129,633,897 | 129,636,342 | 129,636,343 | 129,636,310 | 129,636,925 | 129,641,160 | 129,648,732 |
| 129,652,278 | 129,653,851 | 129,656,420 | 129,656,532 | 129,657,293 | 129,657,294 | 129,657,295 | 129,657,300 | 129,657,304 |
| 129,657,312 | 129,657,315 | 129,657,324 | 129,657,326 | 129,657,327 | 129,657,329 | 129,657,337 | 129,657,353 | 129,664,230 |
| 129,664,232 | 129,664,293 | 129,666,399 | 129,667,149 | 129,667,246 | 129,667,772 | 129,668,316 | 129,669,884 | 129,672,174 |
| 129,672,290 | 129,672,646 | 129,673,523 | 129,673,557 | 129,674,090 | 129,675,341 | 129,676,928 | 129,677,374 | 129,677,629 |
| 129,677,673 | 129,677,709 | 129,678,442 | 129,680,161 | 129,680,629 | 129,686,725 | 129,687,036 | 129,687,164 | 129,687,794 |
| 129,689,537 | 129,690,605 | 129,690,606 | 129,693,550 | 129,693,600 | 129,695,429 | 129,699,101 | 129,699,962 | 129,703,922 |
| 129,704,872 | 129,704,873 | 129,704,991 | 129,705,030 | 129,710,768 | 129,712,713 | 129,712,356 | 129,713,733 | 129,713,737 |
| 129,713,738 | 129,713,739 | 129,717,266 | 129,718,758 | 129,719,237 | 129,719,562 | 129,719,300 | 129,719,820 | 129,720,038 |
| 129,722,574 | 129,722,775 | 129,722,776 | 129,723,383 | 129,723,491 | 129,723,494 | 129,723,497 | 129,729,158 | 129,731,038 |
| 129,733,738 | 129,735,433 | 129,735,730 | 129,736,660 | 129,738,079 | 129,761,662 | 129,773,034 | 129,774,159 | 129,777,886 |
| 129,778,385 | 129,779,295 | 129,779,321 | 129,798,633 | 129,803,712 | 129,804,869 | 129,805,741 | 129,311,014 | 129,812,676 |
| 129,812,778 | 129,812,969 | 129,813,068 | 129,821,519 | 129,827,070 | 129,830,120 | 129,831,773 | 129,831,774 | 129,834,223 |
| 129,837,610 | 129,837,611 | 129,841,661 | 129,841,839 | 129,841,933 | 129,844,962 | 129,846,552 | 129,847,296 | 129,847,546 |
| 129,849,055 | 129,849,662 | 129,850,135 | 129,850,275 | 129,857,182 | 129,864,519 | 129,885,100 | 129,838,841 | 129,889,451 |
| 129,891,425 | 129,893,859 | 130,476,815 | 131,664,348 | 131,667,318 | 131,693,710 | 131,704,736 | 131,706,375 | 131,707,539 |
| 131,726,110 | 131,726,111 | 131,734,265 | 131,842,801 | 131,853,020 | 131,853,184 | 131,854,974 | 131,855,532 | 131,856,051 |
| 131,856,316 | 131,858,650 | 131,859,300 | 131,861,196 | 131,861,243 | 131,861,284 | 131,861,319 | 131,861,545 | 131,861,741 |
| 131,861,783 | 131,861,797 | 131,861,793 | 131,861,829 | 131,861,848 | 131,861,849 | 131,861,850 | 131,861,864 | 131,861,914 |
| 131,862,031 | 131,862,032 | 131,862,422 | 131,862,475 | 131,862,482 | 131,862,601 | 131,862,602 | 131,864,343 | 131,864,629 |
| 131,364,631 | 131,872,529 | 131,872,582 | 131,872,533 | 131,872,534 | 131,372,599 | 131,872,605 | 131,872,607 | 131,872,608 |
| 131,872,641 | 131,873,917 | 131,874,100 | 131,874,290 | l31,874,697 | 131,876,571 | l31,876,623 | 131,876,656 | 131,877,220 |
| 131,877,514 | 131,877,809 | 131,878,335 | 131,878,542 | 131,878,865 | 131,879,236 | 131,880,496 | 131,880,551 | 131,880,593 |
| 131,880,687 | 131,880,733 | 131,880,759 | 131,880,794 | 131,880,804 | 131,881,065 | 131,881,441 | 131,381,809 | 131,881,834 |
| 131,381,910 | 131,882,628 | 131,882,923 | 131,884,410 | 131,885,054 | 131,885,076 | 131,885,077 | 131,885,482 | 131,887,556 |
| 131,837,990 | 131,888,557 | | | | | | | |

TABLE 3
1.24 558 three-valence bismuth (“Bi”) containing compounds were predicted to efficiently bind PANDA Pocket
and efficiently rescue Structural mp53. All of the 94.2 million structures recorded in PubChem
(https://pubchem.ncbi.nlm.nih.gov/) were applied for 4C+ screening. In the 4C+ screening, we collected those with
more than 2 cysteine-binding potential. Carbon-binding As/Sb/Bi bond has defect in binding cysteine since this bond
cannot be hydrolyzed. The other As/Sb/Bi bond can be hydrolyzed in cells and thus is able to bind cysteine.
Bi 3 Valence PANDA Agents

| CID No. | CID No. | CID No. | CID No. | CID No. | CID No. | CID No. | CID No. | CID No. |
|---|---|---|---|---|---|---|---|---|
| 6,879 | 9,010 | 9,242 | 14,776 | 24,591 | 29,573 | 82,232 | 82,233 | 111,041 |
| 111,042 | 273,108 | 409,574 | 438,310 | 554,966 | 560,564 | 3,264,969 | 3,310,373 | 3,353,258 |
| 3,693,500 | 3,826,913 | 3,335,619 | 5,219,607 | 5,351,543 | 6,097,053 | 6,101,599 | 6,327,012 | 6,327,096 |
| 6,327,902 | 6,328,044 | 6,328,058 | 6,328,061 | 6,328,064 | 6,328,110 | 6,328,152 | 6,328,166 | 6,328,191 |
| 6,328,192 | 6,328,644 | 6,328,667 | 6,328,676 | 6,328,741 | 6,329,119 | 6,329,123 | 6,330,910 | 6,330,912 |
| 6,331,875 | 6,332,255 | 6,334,573 | 6,335,194 | 6,335,198 | 6,335,206 | 6,335,254 | 6,335,359 | 6,335,607 |
| 6,336,257 | 6,338,042 | 6,365,054 | 6,365,215 | 6,365,241 | 6,367,060 | 6,367,061 | 6,367,062 | 6,367,063 |
| 6,367,235 | 6,368,739 | 6,369,167 | 6,374,651 | 6,378,840 | 6,379,155 | 6,379,269 | 6,379,609 | 6,381,732 |
| 6,386,980 | 6,391,530 | 6,397,242 | 6,711,587 | 6,832,148 | 6,914,522 | 6,914,523 | 6,914,524 | 6,914,525 |
| 9,804,891 | 9,810,547 | 9,942,004 | 9,987,441 | 10,053,092 | 10,054,529 | 10,246,425 | 10,327,332 | 10,391,773 |
| 10,717,929 | 10,832,460 | 11,049,781 | 11,069,084 | 11,073,147 | 11,146,955 | 11,284,230 | 11,366,706 | 11,411,567 |
| 11,528,063 | 11,643,620 | 11,686,583 | 11,765,348 | 11,786,056 | 11,979,775 | 12,147,504 | 12,622,640 | 13,751,291 |
| 13,766,272 | 13,775,335 | 13,775,336 | 13,828,286 | 13,908,690 | 14,044,341 | 14,044,344 | 14,085,833 | 14,619,600 |
| 14,849,493 | 15,240,258 | 15,240,263 | 15,328,170 | 15,770,557 | 15,770,559 | 15,817,730 | 16,132,801 | 16,132,845 |
| 16,132,857 | 16,132,866 | 16,132,889 | 16,133,169 | 16,212,591 | 16,212,592 | 16,682,734 | 16,682,825 | 16,682,928 |
| 16,682,937 | 16,682,955 | 16,682,959 | 16,682,960 | 16,682,976 | 16,682,977 | 16,682,999 | 16,683,015 | 16,683,017 |
| 16,683,095 | 16,683,098 | 16,683,103 | 16,683,121 | 16,683,563 | 16,683,564 | 16,683,565 | 16,683,566 | 16,683,596 |
| 16,683,686 | 16,683,874 | 16,683,875 | 16,683,958 | 16,683,961 | 16,683,963 | 16,684,025 | 16,684,114 | 16,684,115 |
| 16,684,274 | 16,684,350 | 16,684,468 | 16,684,575 | 16,684,578 | 16,684,580 | 16,684,582 | 16,684,785 | 16,684,795 |
| 16,684,898 | 16,685,021 | 16,685,033 | 16,685,173 | 16,685,257 | 16,685,276 | 16,685,277 | 16,685,391 | 16,685,632 |
| 16,685,998 | 16,686,097 | 16,686,099 | 16,686,175 | 16,686,256 | 16,686,258 | 16,686,506 | 16,687,246 | 16,687,501 |
| 16,688,082 | 16,688,103 | 16,688,295 | 16,688,699 | 16,688,996 | 16,689,505 | 16,689,550 | 16,689,947 | 16,693,013 |
| 16,693,015 | 16,695,048 | 16,695,049 | 16,696,198 | 16,697,869 | 16,697,870 | 16,697,871 | 16,697,873 | 16,699,463 |
| 16,700,590 | 16,700,595 | 16,700,901 | 16,701,354 | 16,701,355 | 16,702.113 | 16,702,492 | 16,702,987 | 16,703,065 |
| 16,704,191 | 16,704,973 | 16,704,976 | 16,704,977 | 16,704,984 | 16,704,997 | 16,705,083 | 16,705,860 | 16,707,734 |
| 16,711,827 | 16,712,566 | 16,716,635 | 16,717,608 | 16,717,608 | 16,717,622 | 16,721,198 | 18,502,954 | 18,503,058 |
| 18,503,101 | 18,503,123 | 13,503,172 | 18,503,263 | 18,503,264 | 18,503,288 | 18,503,517 | 18,503,857 | 18,690,641 |
| 18,690,642 | 21,932,960 | 21,932,966 | 21,932,967 | 21,932,971 | 21,932,934 | 21,932,998 | 21,933,008 | 21,933,012 |
| 21,933,025 | 21,933,028 | 21,933,044 | 21,933,048 | 21,933,092 | 21,933,118 | 21,933,120 | 21,933,124 | 21,933,126 |
| 21,933,129 | 21,933,167 | 21,933,168 | 21,933,169 | 21,933,179 | 21,933,244 | 21,933,252 | 21,933,277 | 21,933,284 |
| 21,933,317 | 21,933,328 | 21,933,329 | 21,933,331 | 21,933,335 | 21,933,358 | 21,933,362 | 21,933,365 | 21,933,367 |
| 21,933,368 | 22,834,097 | 22,834,234 | 22,834,266 | 22,834,406 | 22,834,407 | 22,929,628 | 23,230,204 | 23,576,945 |
| 24,182,960 | 24,184,948 | 24,771,807 | 24,884,204 | 24,884,205 | 25,021,316 | 42,619,967 | 44,135,860 | 44,135,899 |
| 44,146,529 | 44,152,487 | 45,052,077 | 46,245,067 | 50,912,221 | 50,920,714 | 50,920,757 | 50,931,875 | 53,393,588 |
| 53,427,529 | 54,605,444 | 54,742,562 | 56,841,599 | 56,842,096 | 56,842,896 | 56,845,529 | 56,845,924 | 56,846,073 |
| 56,846,074 | 56,846,075 | 56,846,076 | 57,347,031 | 57,357,928 | 57,404,116 | 57,488,589 | 57,562,349 | 58,330,672 |
| 59,499,197 | 59,720,413 | 59,720,414 | 70,294,115 | 71,300,497 | 71,309,993 | 71,310,157 | 71,310,700 | 71,311,500 |
| 71,380,213 | 71,380,459 | 71,386,040 | 71,391,524 | 71,400,325 | 73,557,522 | 73,894,347 | 73,894,349 | 73,894,350 |
| 73,995,040 | 73,995,041 | 76,959,357 | 85,470,850 | 85,470,850 | 85,470,851 | 85,624,350 | 85,750,126 | 87,126,125 |
| 87,207,683 | 87,207,773 | 87,207,774 | 87,238,523 | 87,452,858 | 87,479,654 | 87,489,025 | 87,489,305 | 87,489,320 |
| 87,489,335 | 87,489,593 | 87,489,619 | 87,489,650 | 87,489,777 | 87,490,196 | 87,490,221 | 87,490,477 | 87,490,638 |
| 87,490,646 | 87,490,651 | 87,490,697 | 87,490,956 | 87,490,958 | 87,491,355 | 87,491,335 | 87,491,423 | 87,513,516 |
| 87,513,517 | 87,580,507 | 87,686,514 | 87,686,718 | 87,687,197 | 87,687,712 | 87,693,276 | 87,730,138 | 87,730,829 |
| 87,737,505 | 87,745,214 | 87,871,946 | 87,871,947 | 87,912,467 | 87,912,468 | 87,960,355 | 88,029,006 | 88,029,007 |
| 88,193,567 | 88,194,406 | 88,194,416 | 88,194,477 | 88,194,479 | 88,194,481 | 38,194,539 | 88,194,861 | 88,194,864 |
| 88,194,865 | 88,194,870 | 88,292,939 | 88,292,940 | 88,520,030 | 88,640,276 | 88,640,277 | 88,802,217 | 88,802,218 |
| 88,817,848 | 88,836,364 | 90,471,463 | 90,471,464 | 90,473,777 | 90,473,778 | 90,475,361 | 91,659,154 | 91,886,165 |
| 91,886,166 | 91,886,248 | 91,886,395 | 91,886,591 | 92,003,295 | 92,024,600 | 92,025,641 | 92,025,642 | 92,025,643 |
| 92,025,667 | 92,025,668 | 92,025,669 | 92,025,670 | 92,025,671 | 92,025,673 | 92,025,675 | 92,025,675 | 92,026,749 |
| 92,026,962 | 102,602,549 | 102,602,549 | 117,065,228 | 117,071,502 | 118,855,609 | 118,855,610 | 118,985,207 | 121,233,070 |
| 121,233,129 | 121,233,209 | 121,233,210 | 121,233,689 | 121,233,709 | 121,233,710 | 121,513,973 | 122,173,805 | 122,173,806 |
| 129,627,851 | 129,627,851 | 129,627,852 | 129,627,852 | l29,628,345 | 129,628,507 | l29,630,444 | 129,631,009 | 129,631,020 |
| 129,631,047 | 129,632,182 | 129,632,246 | 129,632,626 | 129,632,968 | 129,634,022 | 129,636,151 | 129,636,165 | 129,636,935 |
| 129,636,953 | 129,637,329 | 129,643,765 | 129,643,766 | 129,644,127 | 129,651,463 | 129,651,702 | 129,651,972 | 129,660,613 |
| 129,661,690 | 129,661,691 | 129,661,692 | 129,662,722 | 129,665,855 | 129,670,802 | 129,671,601 | 129,672,684 | 129,676,730 |
| 129,692,302 | 129,693,004 | 129,693,466 | 129,696,491 | 129,713,086 | 129,713,109 | 129,713,799 | 129,719,991 | 129,722,002 |
| 129,734,931 | 129,738,069 | 129,738,070 | 129,738,237 | 129,759,637 | 129,759,633 | 129,760,053 | 129,760,197 | 129,760,222 |
| 129,760,768 | 129,761,066 | 129,761,789 | 129,761,805 | 129,761,822 | 129,768,269 | 129,769,201 | 129,772,739 | 129,772,785 |
| 129,772,858 | 129,773,065 | 129,793,750 | 129,796,408 | l29,736,934 | 129,802,846 | 129,802,847 | 129,817,421 | 129,819,068 |
| 129,322,257 | 129,822,258 | 129,831,438 | 129,842,132 | 129,842,163 | 129,842,182 | 129,842,230 | 129,842,243 | 129,842,265 |
| 129,842,280 | 129,843,462 | 129,843,543 | 129,843,683 | 129,851,323 | 129,851,730 | 129,8352,640 | 129,856,536 | 129,856,531 |
| l29,865,810 | 129,880,632 | 129,880,679 | 129,887,215 | 129,890,639 | 129,891,194 | 129,891,993 | 131,864,282 | 131,875,075 |

Table 4
1.25 125 five-valence bismuth (“Bi”) structures were predicted to efficiently bind PANDA Pocket and efficiently
rescue Structural mp53. All of the 94.2 million structures recorded in PubChem (https://pubchem.ncbi.nlm.nih.gov/)
were applied for 4C+ screening. In the 4C+ screening, we collected those with more than 2 cysteine-binding
potential. Carbon-binding As/Sb/Bi bond has defect in binding cysteine since this bond cannot be hydrolyzed. The other
As/Sb/Bi bond can be hydrolyzed in cells and thus is able to bind cysteine.
Bi 5 Valence PANDA Agents

| CID No. | CID No. | CID No. | CID No. | CID No. | CID No. | CID No. | CID No. | CID No. |
|---|---|---|---|---|---|---|---|---|
| 82,993 | 123,260 | 366,096 | 366,097 | 366,098 | 366,099 | 367,796 | 434,309 | 438,389 |
| 2,734,035 | 3,301,555 | 3,336,805 | 3,538,901 | 3,653,008 | 3,687,219 | 3,783,338 | 3,914,869 | 3,992,551 |
| 4,063,671 | 4,271,061 | 4,441,160 | 5,254,690 | 5,256,620 | 5,257,028 | 5,257,050 | 5,257,056 | 5,257,058 |
| 5,257,059 | 5,257,062 | 5,257,063 | 6,327,785 | 6,334,091 | 6,372,954 | 6,373,400 | 6,377,431 | 6,386,952 |
| 6,392,480 | 6,392,684 | 6,392,717 | 6,392,777 | 6,394,267 | 6,394,856 | 6,394,889 | 6,395,066 | 6,395,342 |
| 6,395,344 | 6,395,345 | 6,395,477 | 6,396,057 | 6,356,714 | 6,397,027 | 6,397,420 | 6,397,557 | 6,711,667 |
| 6,711,688 | 6,711,693 | 6,850,036 | 6,850,087 | 6,850,113 | 10,907,992 | 11,188,826, | 11,966,239 | 11,968,204 |
| 11,980,596 | 11,980,909 | 11,985,775 | 11,985,985 | 14,420,964 | 16,132,606 | 16,132,606 | 16,132,662 | 16,132,822 |
| 16,132,858 | 16,132,869 | 16,132,960 | 16,332,965 | 16,333,022 | 16,133,325 | 16,133,326 | 16,682,751 | 16,684,917 |
| 16,685,090 | 16,685,106 | 16,685,289 | 16,685,303 | 16,685,390 | 16,685,453 | 16,685,454 | 16,685,455 | 16,685,456 |
| 16,685,573 | 16,685,574 | 16,685,631 | 16,685,637 | 16,715,250 | 18,503,128 | 18,503,206 | 19,032,053 | 21,554,417 |
| 21,554,418 | 21,944,623 | 23,304,890 | 23,304,944 | 23,350,967 | 23,350,968 | 23,633,148 | 23,639,873 | 123,681,527 |
| 23,693,098 | 50,920,763 | 53,249,975 | 58,280.986 | 71,357,908 | 71,401,130 | 87,744,887 | 87,745,037 | 87,745,510 |
| 88,228,697 | 88,515,761 | 88,797,100 | 891,886,318 | 124,202,748 | 129,830,992 | 131,864,281 | 131,868,916 | |

TABLE 5
1.26 937 three-valence antimony (“Sb”) structures were predicted to efficiently bind PANDA Pocket and
efficiently rescue Structural mp53. All of the 94.2 million structures recorded in PubChem
(https://pubchem.ncbi.nlm.nih.gov/) were applied for 4C+ screening. In the 4C+ screening,
we collected those with more than 2 cysteine-binding potential. Carbon-binding As/Sb/Bi
bond has defect in binding cysteine since this bond cannot be hydrolyzed. The other
As/Sb/Bi bond can be hydrolyzed in cells and thus is able to bind cysteine.
Sb 3 Valence PANDA Agents

| CID No. | CID No. | CID No. | CID No. | CID No. | CID No. | CID No. | CID No. | CID No. |
|---|---|---|---|---|---|---|---|---|
| 9,3839 | 18,293 | 24,554 | 24,615 | 24,630 | 24,814 | 27,652 | 46,678 | 61,553 |
| 72,062 | 82,258 | 83,733 | 85,159 | 85,168 | 91,307 | 92,908 | 110,797 | 110,822 |
| 112,343 | 112,417 | 116,971 | 117,333 | 117,654 | 120,130 | 122,445 | 139,227 | 172,734 |
| 201,832 | 223,838 | 231,056 | 273,193 | 278,175 | 292,777 | 432,482 | 443,987 | 2,724,507 |
| 3,022,556 | 3,468,413 | 3,500,394 | 3,980,171 | 4,003,909 | 4,169,194 | 4,227,891 | 4,310,207 | 4,426,282 |
| 4,868,265 | 5,099,079 | 5,205,981 | 5,460,498 | 5,460,499 | 5,475,465 | 6,100,615 | 6,100,855 | 6,102.344 |
| 6,327,063 | 6,327,128 | 6,327,518 | 6,327,644 | 6,327,672 | 6,327,776 | 6,327,782 | 6,327,783 | 6,327,784 |
| 6,327,790 | 6,327,791 | 6,327,792 | 6,327,901 | 6,328,047 | 6,328,136 | 6,328,158 | 6,328,167 | 6,328,183 |
| 6,328,247 | 6,328,381 | 6,328,497 | 6,328,498 | 6,328,604 | 6,328,723 | 6,328,731 | 6,329,083 | 6,329,280 |
| 6,329.469 | 6,330,102 | 6,331.892 | 6,331,893 | 6,331,894 | 6,332,059 | 6,333,990 | 6,334,205 | 6,334,206 |
| 6,335,210 | 6,335,446 | 6,335,642 | 6,335,799 | 6,335,965 | 6,335,967 | 6,335,971 | 6,336,248 | 6,336,250 |
| 6,336,268 | 6,336,294 | 6,336,294 | 6,336,295 | 6,336,297 | 6,336,298 | 6,336,309 | 6,336,310 | 6,336,314 |
| 6,366,315 | 6,336,966 | 6,337,127 | 6,337,192 | 6,338,235 | 6,338,236 | 6,338,237 | 6,338,238 | 6,338,239 |
| 6,338,275 | 6,338,396 | 6,338,574 | 6,366,253 | 6,367,067 | 6,367,121 | 6,367,122 | 6,367,123 | 6,367,297 |
| 6,367,299 | 6,369,653 | 6,371,267 | 6,372,250 | 6,372,531 | 6,373,293 | 6,378,487 | 6,378,591 | 6,379,063 |
| 6,380,952 | 6,383,190 | 6,383,685 | 6,383,702 | 6,386,140 | 6,389,340 | 6,391,662 | 6,391,766 | 6,392,060 |
| 6,393,025 | 6,394,845 | 6,394,846 | 6,394,847 | 6,394,849 | 6,395,364 | 6,395,540 | 6,395,541 | 6,395,542 |
| 6,395,614 | 6,395,616 | 6,397,381 | 6,398,008 | 6,398,524 | 6,398,617 | 6,398,618 | 6,416,685 | 6,437,746 |
| 6,450,406 | 6,450,407 | 6,451,277 | 6,452,001 | 6,857,635 | 6,914,521 | 6,914,522 | 6,914,523 | 6,914,524 |
| 6,914,526. | 6,914,527 | 9,986,376 | 9,988,372 | 10,078,981 | 10,440,350 | 10,508,768 | 10,604,595 | 10,652,047 |
| 10,723,372 | 10,818,460 | 10,876,667 | 10,887,720 | 10,939,671 | 10,953,054 | 11,005,047 | 11,018,607 | 11,028,580 |
| 11,038,092 | 11,039.089 | 11,088,054 | 11,091,142 | 11,181,852 | 11,193,223 | 11,215,665 | 11,216,896 | 11,400,464 |
| 11,416,121 | 11,479,469 | 11,489,983 | 11,542,349 | 11,600,329 | 11,643.503 | 11,801,623 | 11,824,030 | 11,954,289 |
| 11,968,245 | 11,969,034 | 11,979,399 | 12,545,033 | 13,100,649 | 13,165,617 | 13,706,358 | 14,085,828 | 14,085,832 |
| 14,766,829 | 14,923,299 | 15,165,103 | 15,246,215 | 15,274,122 | 15,630,343 | 15,773,247 | 15,779,436 | 15,815,188 |
| 16,132,617 | 16,132,626 | 16,132,842 | 16,132,972 | 16,682,736 | 16,682,737 | 16,682,742 | 16,682,744 | 16,682,747 |
| 16,682,749 | 16,682,752 | 16,682,753 | 16,682,754 | 16,682,822 | 16,682,940 | 16,682,941 | 16,682,985 | 16,682,986 |
| 16,682,996 | 16,683,002 | 16,683,008 | 16,683,009 | 16,683,047 | 16,683,067 | 16,683,068 | 16,683,082 | 16,683,091 |
| 16,683,110 | 16,683,184 | 16,683,603 | 16,683,605 | 16,683,607 | 16,683,654 | 16,683,656 | 16,683,857 | 16,683,859 |
| 16,683,966 | 16,683,967 | 16,684,084 | 16,684,126 | 16,684,127 | 16,684,162 | 16,684,169 | 16,684,206 | 16,684,210 |
| 16,684,264 | 16,684,266 | 16,684,267 | 16,684,268 | 16,684,270 | 16,684,295 | 16,684,303 | 16,684,309 | 16,684,377 |
| 16,684,378 | 16,684,379 | 16,684,380 | 16,684,381 | 16,684,383 | 16,684,474 | 16,684,490 | 16,684,491 | 16,684,542 |
| 16,684,587 | 16,684,616 | 16,684,617 | 16,684,618 | 16,684,619 | 16,684,620 | 16,684,622 | 16,684,713 | 16,684,714 |
| 16,684,728 | 16,684,732 | 16,684,860 | 16,684,861 | 16,684,878 | 16,684,889 | 16,685,013 | 16,685,014 | 16,685,015 |
| 16,685,080 | 16,685,138 | 16,685,151 | 16,684,153 | 16,685,185 | 16,685,197 | 16,685,221 | 16,685,265 | 16,685,272 |
| 16.685,273 | 16,685,311 | 16,685,415 | 16,685,416 | 16,685,417 | 16,685,418 | 16,685,479 | 16,685,481 | 16,685,497 |
| 16,685,764 | 16,686,007 | 16,686,173 | 16,686,176 | 16,686,177 | 16,686,294 | 16,686,575 | 16,686,576 | 16,687,650 |
| 16,687,895 | 16,688,146 | 16,683,454 | 16,688,473 | 16,688,527 | 16,688,544 | 16,688,545 | 16,688,698 | 16,688,732 |
| 16,688,935 | 16,688,973 | 16,688,974 | 16,688,975 | 16,689,006 | 16,689,163 | 16,689,752 | 16,691,582, | 16,693,615 |
| 16,693,637 | 16,693,639 | 16,693,641 | 16,694.223 | 16,695,174 | 16,695,175 | 16,695,950 | 16,695,951 | 16,695,952 |
| 16,697,056 | 16,697,515 | 16,700,667 | 16,700,668 | 16,700,670 | 16,700,811 | 16,700,812 | 16,700,896 | 16,701,020 |
| 16,703,714 | 16,705,235 | 16,706,597 | 16,708,103 | 16,70S,935 | 16,709,013 | 16,709,919 | 16,711,745 | 16,712,315 |
| 16,713,256 | 16,716,705 | 16,738,697 | 16,760,657 | 17,749,634 | 17,757,230 | 17,757,257 | 17,896,354 | 17,907,305 |
| 18,347,240 | 18,502,908 | 13,502,961 | 18,502,962 | 18,503,115 | 18,503,122 | IS,503,252 | 18,503,253 | 18,503,254 |
| 18,503,313 | 18,503,565 | 18,503,729 | 18,503,753 | 18,690,650 | 18,690,654 | IS,690,656 | 13,690,658 | 19,097,033 |
| 19,097,037 | 19,821,447 | 19,933,062 | 20,111,700 | 20,185,678 | 20,249,102 | 20,249,105 | 20,249,108 | 20,249,113 |
| 20,249,115 | 20,300,474 | 20,313,258 | 20,315,019 | 20,363,281 | 20,435,550 | 20,650,165 | 20,650,168 | 20,650,130 |
| 20,836,036 | 20,838,936 | 20,840,786 | 20,840,787 | 20,841,413 | 20,841,414 | 21,127,957 | 21,127,960 | 21,162,914 |
| 21,162,915 | 21,162,916 | 21,179,954 | 21,280,137 | 21,387,935 | 21,429,702 | 21,431,092 | 21,597,739 | 21,597,740 |
| 21,597,742 | 21,597,743 | 21,597,747 | 21,853,875 | 21,853,876 | 21,853,877 | 21,853,378 | 21,853,880 | 21,853,882 |
| 21,853,384 | 21,924,199 | 21,946,953 | 21,976,042 | 22,134,008 | 22,476,815 | 22,483,778 | 22,755,405 | 22,755,406 |
| 22,755,407 | 22,755,408 | 22,755,409 | 22,755,410 | 22,834,430 | 23,089,770 | 23,232,432 | 23,262,264 | 23,262,275 |
| 23,262,289 | 23,262,291 | 23,262,328 | 23,262,329 | 23,271,407 | 23,271,424 | 23,271,440 | 23,271,479 | 23,412,698 |
| 23,412,699 | 23,412,700 | 23,412,701 | 23,412,702 | 23,412,703 | 23,412,704 | 23,412,705 | 23,412,706 | 23,412,707 |
| 23,412,708 | 23,412,709 | 23,412,710 | 23,412,711 | 23,412,744 | 23,412,745 | 23,412,746 | 23,412,747 | 23,424,127 |
| 23,452,096 | 23,617,918 | 23,665,405 | 23,667,272 | 23,675,780 | 23,681,183 | 23,686,990 | 23,690,288 | 23,707,960 |
| 23,714,520 | 24,182,330 | 24,182,331 | 24,199,786 | 24,201,065 | 24,837,728 | 24,847,360 | 24,867,558 | 24,884,257 |
| 25,021,693 | 25,200,065 | 44,119,133 | 44,135,767 | 44,135,895 | 44,145,400 | 44,145,839 | 44,150,046 | 44,153,415 |
| 45,479,364 | 45,479,524 | 45,479,539 | 50,896,902 | 50,909,120 | 50,918,374 | 50,921,100 | 50,930,621 | 50,933,843 |
| 50,935,021 | 53,315,432 | 53,471,862 | 53,494,194 | 54,603,506 | 54,604,975 | 54,605,443 | 54,611,195 | 54,688,499 |
| 54,703,985 | 54,703,986 | 54,703,987 | 54,724,826 | 54,742,027 | 54,750,834 | 56,845,640 | 56,927,675 | 57,347,421 |
| 57,348,872 | 57,350,497 | 57,352,871 | 57,357,960 | 57,370,241 | 57,371,215 | 57,490,232 | 57,645,580 | 57,704,204 |
| 57,704,207 | 57,731,111 | 57,731,115 | 57,731,116 | 57,731,118 | 57,731,120 | 57,731,121 | 57,731,122 | 57,789,471 |
| 57,789,472 | 57,953,647 | 58,253,024 | 58,253,026 | 58,253,027 | 58,253,029 | 58,253,030 | 58,253,031 | 58,253,032 |
| 58,271,553 | 58,271,555 | 58,271,564 | 58,271,575 | 58,271,579 | 58,271,583 | 58,280,987 | 58,280,983 | 58,280,990 |
| 58,288,679 | 58,609,137 | 58,720,422 | 59,032,477 | 53,086,320 | 59,159,833 | 59,159,383 | 59,499,187 | 59,499,195 |
| 59,499,196 | 59,499,199 | 59,499,204 | 59,557,632 | 59,571,971 | 59,571,972 | 59,891,573 | 59,891,601 | 59,891,603 |
| 59,891,640 | 71,301,022 | 71,301,023 | 71,301,024 | 71,333,883 | 71,342,634 | 71,345,945 | 71,345,945 | 71,345,946 |
| 71,359,975 | 71,361,093 | 71,363,456 | 71,367,105 | 71,374,340 | 71,380,591 | 71,429,674 | 71,441,094 | 71,442,382 |
| 72,720,464 | 72,720,4681 | 73,307,702 | 73,307,769 | 73,307,770 | 73,307,825 | 73,307,873 | 73,555,373 | 73,555,376 |
| 73,555,379 | 73,555,501 | 73,555,893 | 73,557,535 | 73,759,938 | 73,894,305 | 73,894,308 | 73, 894,311 | 73,394,312 |
| 73,894,313 | 73,394,314 | 73,894,315 | 73,894,323 | 73,952,085 | 73,995,019 | 73,995,020 | 74,040,665 | 74,765,653 |
| 74,933,683 | 74,935,384 | 76,037,526 | 76,960,497 | 85,551,321 | 85,609,459 | 85,618,045 | 85,750,126 | 85,750,126 |
| 35,863,651 | 85,863,652 | 85,907,651 | 85,976,002 | 86,101,760 | 86,101,767 | 86,101,784 | 86,246,892 | 87,138,989 |
| 87,186,483 | 87,202,814 | \|87,258,578 | 87,261,615 | 87,261,620 | 87,261,624 | 87,261,630 | 87,261,631 | 87,261,715 |
| 87,261,713 | 87,261,720 | 87,261,724 | 87,261,726 | 87,261,729 | 87,261,732 | 87,261,737 | 87,261,749 | 87,261,752 |
| 87,315,219 | 87,315,760 | 87,362,495 | 87,373,499 | 87,411,324 | 87,438,483 | 87,438,929 | 87,450,539 | 87,460,730 |
| 87,635,564 | 87,702,251 | 87,719,536 | 87,719,533 | 87,835,653 | 87,857,783 | 88,154,076 | 88,176,411 | 88,176,414 |
| 88,261,066 | 88,261,223 | 88,264,017 | 88,264,710 | 88,265,017 | 88,265,019 | 88,423,296 | 88,430,055 | 88,466,104 |
| 88,473,381 | 88,484,275 | 88,484,276 | 88,526,235 | 88,526,236 | 88,526,238 | 88,526,252 | 88,526,253 | 88,613,920 |
| 88,613,921 | 88,642,592 | 88,654,956 | 88,654,957 | 88,745,663 | 88,772,726 | 88,773,222 | 88,773,337 | 88,774,019 |
| 88,774,061 | 88,777,596 | 88,778,787 | 88,793,603 | 88,793,935 | 88,795,315 | 88,795,398 | 88,800,757 | 88,801,147 |
| 88,801,216 | 88,806,743 | 88,806,751 | 88,820,185 | 88,824,737 | 90,105,233 | 90,105,284 | 90,471,546 | 90 473,139 |
| 90,659,538 | 90,659,541 | 90,659,637 | 91,666,614 | 91,667,987 | 91,668,102 | 91,867,127 | 91,868,149 | 91,886,487 |
| 91,980,813 | 91,980,814 | 91,996,065 | 91,997,283 | 91,997,284 | 91,997,363 | 91,997,683 | 92,004,432 | 92,012,267 |
| 92,012,268 | 92,023,437 | 92,024,179 | 92,024,528 | 92,024,529 | 92,024,531 | 92,024,534 | 92,024,536 | 92,024,556 |
| 92,024,935 | 92,024,944 | 92,024,955 | 92,024,957 | 92,024,958 | 92,024,961 | 92,024,968 | 92,024,970 | 92,024,971 |
| 92,024,972 | 92,024,973 | 92,024,976 | 92,024,977 | 92,024,980 | 92,024,981 | 92,024,982 | 92,024,985 | 92,024,986 |
| 92,024,987 | 92,024,983 | 92,024,990 | 92,024,991 | 92,024,997 | 92,025,001 | 92,025,002 | 32,025,003 | 92,025,020 |
| 92,025,024 | 92,025,025 | 92,025,026 | 92,025,027 | 92,025,028 | 92,025,031 | 92,025,050 | 92,025,051 | 92,025,052 |
| 92,025,053 | 92,025,054 | 92,025,055 | 92,025,056 | 92,025,057 | 92,025,058 | 92,025,059 | 92,025,063 | 92,025,064 |
| 92,025,065 | 92,025,066 | 92,025,067 | 92,025,069 | 92,025,077 | 92,025,079 | 92,025,082 | 92,025,083 | 92,025,084 |
| 92,025,035 | 92,025,086 | 92,025,087 | 92,025,089 | 92,028,426 | 92,028,427 | 92,028,428 | 92,028,429 | 92,028,430 |
| 92,028,791 | 92,028,940 | 92,043,602 | 92,043,603 | 102,602,109 | 117,065,228 | 121,233,627 | 121,233,623 | 121,596,016 |
| 123,132,499 | 129,628,368 | 129,628,369 | 129,623,470 | 129,630,336 | 129,631,714 | 129,634,074 | 129,634,980 | 129,635,876 |
| 129,635,919 | 129,636,319 | 129,636,320 | 129,636,621 | 129,636,622 | 129,636,709 | 129,639,940 | 129,646,351 | 129,649,988 |
| 129,664,755 | 129,668,929 | 129,671,029 | 129,671,731 | 129,671,732 | 129,672,175 | 129,672,177 | 129,672,415 | 129,572,416 |
| 129,675,055 | 129,676,925 | 129,677,475 | 129,630,159 | 129,630,177 | 129,680,187 | 129,691,639 | 129,691,640 | 129,697,639 |
| 129,703,076 | 129,709,779 | 129,718,867 | 129,719,189 | 129,721,729 | 129,723,651 | 129,731,772 | 129,731,773 | 129,760,451 |
| 129,765,173 | 129,765,222 | 129,771,692 | 129,773,098 | 129,776,810 | 129,781,425 | 129,783,529 | 129,783,530 | 129,794,056 |
| 129,798,979 | 129,802,873 | 129,807,640 | 129,809,238 | 129,812,117 | 129,814,318 | 129,814,352 | 129,314,419 | 129,817,665 |
| 129,317,666 | 129,821,032 | 129,821,963 | 129,831,304 | 129,842,135 | 129,842,264 | 129,843,508 | 129,343,687 | 129,848,944 |
| 129,352,227 | 129,856,127 | 129,856,231 | 129,864,503 | 129,865,814 | 129,379,550 | 129,879,554 | 129,886,985 | 129,888,228 |
| 129,893,705 | 129,893,706 | 130,476,776 | 131,706,152 | 131,707,327 | 131,852,287 | 131,880,394 | 131,832,596 | 131,882,949 |
| 131,835,062 | | | | | | | | |

TABLE 6
1.27 1896 five-valence antimony (“Sb”) structures were predicted to efficiently bind PANDA Pocket and
efficiently rescue Structural mp53. An of the 94.2 million structures recorded in PubChem
(https://pubchem.ncbi.nlm.nih.govi)were applied for 4C+ screening. In the 4C+ screening,
we collected those with more than 2 cysteine-binding potential. Carbon-binding As/Sb/Bi
bond has defect in binding cysteine since this bond cannot be hydrolyzed. The other
As/SbilBi bond can be hydrolyzed in cells and thus is able to bind cysteine.PD
SB 5 Valence PANDA Agents

| CID No. | CID No. | CID No. | CID No. | CID No. | CID No. | CID No. | CID No. | CID No. |
|---|---|---|---|---|---|---|---|---|
| 11,135 | 14,813 | 24,294 | 24,557 | 25,463 | 25,470 | 28,362 | 50,592 | 61,636 |
| 64,953 | 74,002 | 93,820 | 95,060 | 116,495 | 150,258 | 157,275 | 182,263 | 224,879 |
| 324,882 | 224,884 | 224,885 | 224,886 | 224,889 | 224,895 | 224,898 | 224,899 | 224,900 |
| 224,905 | 224,909 | 224,910 | 224,913 | 224,915 | 224,919 | 224,926 | 224,930 | 224,931 |
| 225,717 | 225,790 | 225,792 | 225,794 | 225,795 | 225,796 | 225,805 | 226,567 | 227,253 |
| 240,550 | 271,854 | 279,133 | 279,142 | 279,144 | 279,147 | 284,456 | 299,579 | 326,416 |
| 408,518 | 408,544 | 408,739 | 408,759 | 411,587 | 420,950 | 420,951 | 420,952 | 420,953 |
| 420,954 | 420,955 | 428,747 | 432,515 | 436,771 | 456,326 | 2,751,968 | 2,777,253 | 3,032,643 |
| 3,081,396 | 3,246,048 | 3,695,004 | 3,868,826 | 3,891,403 | 3,959,835 | 4,349,542 | 4,591,706 | 5,027,238 |
| 5,231,914 | 5,246,643 | 5,311,441 | 5,351,653 | 5,354,627 | 5,362,453 | 5,463,897 | 5,464,034 | 5,464,322 |
| 5,464,511 | 5,771,529 | 6,093,409 | 6,326,778 | 6,327,011 | 6,328,579 | 6,328,716 | 6,328,740 | 6,331,889 |
| 6,331,890 | 6,331,891 | 6,331,835 | 6,331,896 | 6,331,897 | 6,331,398 | 6,331,899 | 6,331,900 | 6,331,902 |
| 6,331,903 | 6,331,905 | 6,332,020 | 6,333,908 | 6,333,910 | 6,335,289 | 6,338,392 | 6,367,296 | 6,367,298 |
| 6,368,256 | 6,373,292 | 6,374,203 | 6,377,210 | 6,377,218 | 6,383,299 | 6,333,671 | 6,336,841 | 6,387,196 |
| 6,390,184 | 6,390,186 | 6,392,009 | 6,392,668 | 6,393,991 | 6,394,511 | 6,394,680 | 6,394,681 | 6,395,057 |
| 6,395,059 | 6,395,115 | 6,396,413 | 6,396,421 | 6,396,442 | 6,396,719 | 5,396,810 | 6,397,382 | 6,397,391 |
| 6,397,717 | 6,393,204 | 6,857,634 | 6,857,636 | 9,810,717 | 9,885,438 | 9,915,631 | 10,090,090 | 10,090,091 |
| 10,259,654 | 10,939,670 | 10,960,282 | 10,960,305 | 11,005,470 | 11,118,066 | 11,297,036 | 11,318,239 | 11,730,658 |
| 11,765,685 | 11,768,112 | 11,807,053 | 11,966,274 | 11,966,275 | 11,980,764 | 11,985,984 | 12,591,815 | 12,725,709 |
| 13,047,299 | 13,467,894 | 13,678,820 | 13,678,826 | 13,751,471 | 13,783,183 | 13,788,547 | 13,956,203 | 14,085,961 |
| 14,324,919 | 15,238,731 | 15,338,575 | 15,396,527 | 15,531,077 | 15,531,078 | 15,829,268 | 15,845,290 | 15,951,499 |
| 16,132,666 | 16,132,670 | 16,132,838 | 16,132,942 | 16,133,190 | 16,133,363 | 16,133,379 | 16,211,378 | 16,211,954 |
| 16,213,713 | 16,656,342 | 16,683,012 | 16,683,013 | 16,683,083 | 16,683,100 | 16,683,108 | 16,683,119 | 16,683,598 |
| 16,683,599 | 16,683,601 | 16,583,608 | 16,683,609 | 16,683,611 | 16,633,513 | 16,683,614 | 16,683,616 | 16,683,618 |
| 16,683,620 | 16,683,62 | 16,683,623 | 16,683,653 | 16,683,881 | 16,683,992 | 16,634,366 | 16,684,410 | 16,684,725 |
| 16,684,726 | 16,684,730 | 16,685,150 | 16,685,183 | 16,685,196 | 16,685,245 | 16,685,271 | 16,685,394 | 16,685,395 |
| 16,685,549 | 16,685,550 | 16,685,551 | 16,685,552 | 16,685,588 | 16,685,683 | 16,685,687 | 16,685,688 | 16,686,075 |
| 16,686,100 | 16,686,112 | l6,686,186 | 16,686,196 | 16,686,225 | 16,686,229 | 16,686,232 | 16,686,233 | 16,686,253 |
| 16,686,296 | 16,686,298 | 16,686,321 | 16,686,393 | 16,686,395 | 16,686,397 | 16,686,399 | 16,686,401 | 16,686,432 |
| 16,686,484 | 16,686,517 | 16,686,548 | 16,686,570 | 16,686,603 | 16,686,636 | 16,686,659 | 16,606,663 | 16,636,674 |
| 16,686,677 | 16,686,683 | 16,686,685 | 16,686,688 | 16,686,700 | 16,686,707 | 16,686,709 | 16,686,711 | 16,686,714 |
| 16,686,716 | 16,686,719 | 16,686,722 | 16,686,723 | 16,686,725 | 16,686,730 | 16,686,754 | 16,686,756 | 16,686,763 |
| 16,686,767 | 16,686,739 | 16,686,792 | 16,686,794 | 16,686,796 | 16,636,793 | 16,636,802 | 16,686,808 | 16,686,810 |
| 16,686,812 | 16,686,816 | 16,686,830 | 16,686,841 | 16,686,843 | 16,686,846 | 16,686,848 | 16,686,854 | 16,686,855 |
| 16,686,860 | 16,686,870 | 16,686,878 | 16,686,890 | 16,686,893 | 16,686,895 | 16,686,898 | 16,686,900 | 16,686,904 |
| 16,686,907 | 16,686,909 | 16,686,911 | 16,686,913 | 16,686,917 | 16,686,921 | 16,686,928 | 16,686,930 | 16,686,932 |
| 16,686,937 | 16,686,939 | 16,686,941 | 16,686,943 | 16,686,948 | 16,686,950 | 16,686,952 | 16,686,954 | 16,686,957 |
| 16,686,959 | 16,636,963 | 16,636,977 | 16,686,979 | 16,686,932 | 16,686,983 | 16,686,986 | 16,686,983 | 16,636,991 |
| 16,687,010 | 16,637,016 | 16,687,021 | 16,687,023 | 16,687,026 | 16,687,029 | 16,687,034 | 16,687,033 | 16,687,043 |
| 16,687,044 | 16,687,053 | 16,687,055 | 16,687,059 | 16,687,061 | 16,687,063 | 16,637,066 | 16,687,069 | 16,687,071 |
| 16,687,099 | 16,687,102 | 16,587,107 | 16,687,110 | 16,687,112 | 16,687,115 | 16,637,119 | 16,687,121 | 16,687,125 |
| 16,687,140 | 16,687,142 | 16,587,144 | 16,687,148 | 16,687,151 | 16,637,155 | 16,637,166 | 16,687,174 | 16,687,177 |
| 16,687,180 | 16,637,183 | 16,687,185 | 16,687,187 | 16,637,189 | 16,687,191 | 16,637,218 | 16,687,220 | 16,687,222 |
| 16,687,224 | 16,687,226 | 16,687,232 | 16,687,234 | 16,687,256 | 16,687,259 | 16,687,261 | 16,687,263 | 16,687,268 |
| 16,687,286 | 16,687,298 | 16,687,306 | 16,687,332 | 16,687,334 | 16,687,339 | 16,687,354 | 16,687,371 | 16,687,376 |
| 16,687,390 | 16,637,403 | 16,637,417 | 16,687,458 | 16,687,461 | 16,687,464 | 16,687,465 | 16,687,471 | 16,637,475 |
| 16,687,482 | 16,687,483 | 16,687,518 | 16,687,546 | 16,687,556 | 16,687,558 | 16,687,566 | 16,687,573 | 16,637,608 |
| 16,687,610 | 16,687,630 | 16,687,631 | 16,687,633 | 16,687,642 | 16,687,648 | 16,637,649 | 16,687,666 | 16,687,668 |
| 16,687,675 | 16,687,677 | 16,687,684 | 16,687,688 | 16,687,717 | 16,637,722 | 16,637,726 | 16,687,741 | 15,687,743 |
| 16,687,765 | 16,687,796 | 16,687,800 | 16,687,815 | 16,637,828 | 16,687,833 | 16,637,837 | 16,687,872 | 16,687,875 |
| 16,687,876 | 16,687,894 | 16,687,398 | 16,687,912 | 16,637,918 | 16,687,923 | 16,637,925 | 16,687,931 | 16,687,934 |
| 16,687,936 | 16,687,940 | 16,687,962 | 16,687,968 | 16,687,972 | 16,687,982 | 16,687,985 | 16,687,990 | 16,688,008 |
| 16,688,015 | 16,688,017 | 16,638,019 | 16,688,023 | 16,688,027 | 16,683,032 | 16,583,037 | 16,688,044 | 16,688,052 |
| 16,688,060 | 16,638,062 | 16,638,085 | 16,688,119 | 16,683,127 | 16,683,128 | 16,688,132 | 16,688,150 | 16,638,155 |
| 16,688,161 | 16,688,166 | 16,688,171 | 16,688,175 | 16,683,181 | 16,688,199 | 16,688,203 | 16,688,204 | 16,638,213 |
| 16,688,236 | 16,688,237 | 16,688,238 | 16,688,242 | 16,688,246 | 16,688,273 | 16,688,277 | 16,688,296 | 16,688,304 |
| 16,688,309 | 16,688,313 | 16,688,340 | 16,688,344 | 16,688,345 | 16,688,347 | 16,688,354 | 16,688,358 | 16,688,360 |
| 16,688,361 | 16,688,378 | 16,688,385 | 16,688,391 | 16,638,396 | 16,688,400 | 16,688,451 | 16,688,470 | 16,688,480 |
| 16,688,481 | 16,688,483 | 16,688,490 | 16,688,492 | 16,688,502 | 16,688,509 | 16,688,511 | 16,688,513 | 16,688,514 |
| 16,688,522 | 16,688,529 | 16,688,534 | 16,688,538 | 16,688,546 | 16,688,549 | 16,688,551 | 16,688,564 | 16,688,571 |
| 16,688,572 | 16,688,581 | 16,688,584 | 16,688,600 | 16,688,605 | 16,688,607 | 16,688,612 | 16,688,523 | 16,638,630 |
| 16,688,633 | 16,688,640 | 16,638,641 | 16,688,650 | 16,688,674 | 16,683,678 | 16,683,722 | 16,688,726 | 16,688,729 |
| 16,688,731 | 16,688,735 | 16,688,740 | 16,688,742 | 16,688,744 | 16,688,746 | 16,688,769 | 16,688,787 | 16,688,789 |
| 16,688,793 | 16,688,804 | 16,688,811 | 16,688,822 | 16,688,326 | 16,688,823 | 16,688,834 | 16,688,841 | 16,688,854 |
| 16,688,859 | 15,688,866 | 16,588,871 | 16,688,373 | 16,688,874 | 16,638,875 | 16,688,888 | 16,688,899 | 16,688,903 |
| 16,688,904 | 16,688,907 | 16,688,910 | 16,688,911 | 16,688,954 | 16,688,968 | 16,638,976 | 16,689,000 | 16,689,003 |
| 16,689,020 | 16,689,026 | 16,689,028 | 16,689,030 | 16,689,032 | 16,689,034 | 16,689,035 | 16,689,039 | 16,689,043 |
| 16,689,066 | 16,689,069 | 16,689,074 | 16 689,091 | 16,689,122 | 16,689,148 | 16,689,159 | 16,689,180 | 16,689,193 |
| 16,689,197 | 16,639,229 | 16,689,244 | 16,689,275 | 16,689,232 | 16,689,288 | 16,689,289 | 16,689,296 | 16,639,306 |
| 16,689,321 | 16,689,323 | 16,689,331 | 16,689,333 | 16,689,362 | 16,689,378 | 16,689,389 | 16,689,407 | 16,689,423 |
| 16,689,425 | 16,689,423 | 16,689,429 | 16,689,433 | 16,689,435 | 16,689,438 | 16,689,447 | 16,689,486 | 16,639,496 |
| 16,689,500 | 16,689,506 | 16,689,509 | 16,689,515 | 16,689,513 | 16,639,523 | 16,689,524 | 16,689,527 | 16,689,528 |
| 16,689,562 | 16,689,563 | 16,689,564 | 16,689,565 | 16,689,571 | 16,689,573 | 16,689,534 | 16,689,586 | 16,689,611 |
| 16,689,613 | 16,689,622 | 16,689,627 | 16,689,630 | 16,689,632 | 16,689,635 | 16,689,637 | 16,689,640 | 16,689,647 |
| 16,689,663 | 16,689,666 | 16,689,673 | 16,689,678 | 16,689,683 | 16,689,685 | 16,689,691 | 16,689,695 | 16,689,727 |
| 16,689,729 | 16,689,736 | 16,689,738 | 16,689,740 | 16,689,741 | 16,689,753 | 16,689,755 | 16,689,758 | 16,689,760 |
| 16,690,353 | 16,692,259 | 16,693,842 | 16,693,976 | 16,694,718 | 16,694,762 | 16,695,738 | 16,696,653 | 16,696,654 |
| 16,696,933 | 16,637,139 | 16,697,406 | 16,697,408 | 16,697,957 | 16,657,959 | 16,697,961 | 16,698,179 | 16,699,328 |
| 16,699,521 | 16,700,362 | 16,700,586 | 16,700,907 | 16,700,908 | 16,701,189 | 16,701,408 | 16,701,417 | 16,701,539 |
| 16,702,362 | 16,702,435 | 16,703,268 | 16,703,361 | 16,703,710 | 16,704,622 | 16,705,392 | 16,705,462 | 16,705,463 |
| 16,705,466 | 16,705,531 | 16,705,533 | 16,705,535 | 16,705,545 | 16,705,675 | 16,706,113 | 16,706,116 | 16,706,118 |
| 16,706,119 | 16,706,769 | 16,707,377 | 16,707,993 | 16,708,818 | 16,710,497 | 16,710,763 | 16,711,795 | 16,711,797 |
| 16,715,788 | 16,715,938 | 16,717,102 | 16,717,107 | 16,717,536 | 16,717,578 | 16,717,583 | 16,717,592 | 16,717,593 |
| 16,717,595 | 16,717,603 | 16,717,605 | 16,717,643 | 15,717,644 | 16,717,652 | 16,717,657 | 16,717,578 | 16,726,724 |
| 16,726,725 | 16,726,928 | 16,727,069 | 16,742,676 | 16,750,638 | 16,750,700 | 16,750,702 | 16,750,704 | 16,750,706 |
| 16,751,742 | 16,752,035 | 16,752,037 | 16,752,039 | 16,752,157 | 16,752,159 | 16,752,280 | 16,752,282 | 16,752,284 |
| 16,752,384 | 16,752,386 | 16,752,388 | 16,752,390 | 16,752,503 | 16,752,505 | 16,752,507 | 16,752,509 | 16,760,656 |
| 17,757,229 | 17,757,914 | 17,764,892 | 17,778,956 | 17,778,357 | 17,736,555 | 17,786,556 | 17,817,953 | 17,822,508 |
| 17,822,510 | 17,826,684 | 17,849,914 | 17,884,946 | 17,897,207 | 17,917,039 | 17,936,221 | 17,945,298 | 17,964,729 |
| 17,976,351 | 18,000,152 | 18,006,734 | 18,182,969 | 18,350,096 | 18,350,179 | 13,350,195 | 18,350,251 | 18,350,260 |
| 18,350,272 | 18,352,342 | 18,352,411 | 18,352,412 | 18,362,431 | 18,364,093 | 18,364,110 | 18,369,789 | 18,377,198 |
| 18,387,057 | 18,387,053 | 18,337,060 | 18,387,072 | 18,401,200 | 18,458,528 | 18,453,565 | 18,468,794 | 18,502,979 |
| 18,503,509 | 18,503,583 | 18,503,597 | 18,503,607 | 18,503,614 | 18,503,626 | 18,503,641 | 18,503,663 | 18,503,674 |
| 18,503,676 | 18,503,678 | 18,503,713 | 18,503,717 | 18,503,718 | 18,503,720 | 18,503,730 | 18,503,732 | 18,503,745 |
| 18,503,756 | 18,503,760 | 18,503,762 | 18,503,780 | 18,503,786 | 18,503,788 | 18,503,795 | 18,503,809 | 18,503,817 |
| 18,503,829 | 18,503,834 | 18,503,854 | 18,503,855 | 18,503,866 | 18,513,362 | 18,513,368 | 18,513,395 | 18,517,340 |
| 18,517,342 | 18,517,343 | 18,533,808 | 18,619,949 | 18,620,401 | 18,698,857 | 18,698,358 | 18,698,859 | 18,698,860 |
| 18,719,776 | 18,755,853 | 13,759,325 | 13,764,251 | 18,764,466 | 18,794,972 | 18,801,121 | 18,801,139 | 18,931,796 |
| 18,951,198 | 18,954,064 | 18,963,165 | 18,968,263 | 18,972,203 | 18,981,572 | 18,982,367 | 18,982,494 | 18,982,517 |
| 13,986,400 | 18,986,401 | 19,019,300 | 19,042,246 | 19,044,994 | 19,045,026 | 19,068,398 | 19,073,968 | 19,073,976 |
| 13,083,438 | 19,093,335 | 19,097,031 | 19,097,032 | 19,097,034 | 19,097,036 | 19,347,784 | 19,349,306 | 19,351,976 |
| 19,354,077 | 19,366,336 | 19,366,341 | 19,366,346 | 19,366,352 | 19,372,168 | 19,372,178 | 19,373,839 | 19,373,841 |
| 19,373,847 | 19,373,849 | 19,379,549 | 19,417,354 | 19,432,230 | 19,594,006 | 19,594,014 | 19,594,015 | 19,594,016 |
| 19,609,633 | 19,638,871 | 19,700,212 | 19,734,742 | 19,734,744 | 19,734,745 | 19,739,452 | 19,748,302 | 19,762,749 |
| 19,762,750 | 19,762,751 | 19,762,752 | 19,762,753 | 19,762,755 | 19,774,966 | 19,795,275 | 19,795,276 | 19,795,277 |
| 19,795,280 | 19,795,286 | 19,795,293 | 19,795,298 | 19,795,302 | 19,821,446 | 19,840,059 | 19,877,071 | 19,887,685 |
| 19,887,689 | 19,887,691 | 19,887,696 | 19,887,720 | 19,887,725 | 19,899,974 | 19,913,507 | 19,969,014 | 19,969,017 |
| 19,969,018 | 19,969,019 | 19,969,021 | 19,969,022 | 19,969,023 | 19,969,024 | 19,969,026 | 19,969,027 | 19,969,031 |
| 19,969,032 | 19,969,036 | 19,969,037 | 19,969,039 | 19,969,040 | 19,969,041 | 19,969,042 | 19,969,043 | 19,971,369 |
| 19,975,042 | 19,981,557 | 19,983,424 | 19,991,813 | 19,993,400 | 19,993,402 | 19,993,412 | 19,993,418 | 20,038,203 |
| 20,056,484 | 20,056,601 | 20,063,739 | 20,063,745 | 20,083,778 | 20,084,143 | 20,084,144 | 20,084,145 | 20,146,627 |
| 20,146,628 | 20,186,109 | 20,195,292 | 20,235,371 | 20,259,845 | 20,291,586 | 20,291,592 | 20,314,491 | 20,391,574 |
| 20,401,169 | 20,443,249 | 20,443,251 | 20,463,996 | 20,474,401 | 20,474,407 | 20,474,432 | 20,474,444 | 20,493,173 |
| 20,529,305 | 20,829,307 | 20,529,309 | 20,549,661 | 20,554,899 | 20,562,912 | 20,658,850 | 20,669,215 | 20,670,558 |
| 20,835,778 | 20,835,967 | 20,836,032 | 20,836,033 | 20,836,037 | 20,836,063 | 20,836,064 | 20,836,065 | 20,836,066 |
| 20,S36,067 | 20,336,069 | 20,841,651 | 20,846,195 | 20,846,196 | 20,981,344 | 20,931,345 | 20,981,346 | 20,981,347 |
| 21,096,833 | 21,113,938 | 21,119,588 | 21,126,179 | 21,127,108 | 21,127,109 | 21,139,532 | 21,139,533 | 21,188,886 |
| 21,188,887 | 21,183,889 | 21,188,390 | 21,201,179 | 21,263,248 | 21,287,599 | 21,290,433 | 21,292,545 | 21,292,548 |
| 21,523,241 | 21,536,746 | 21,646,515 | 21,646,518 | 21,646,549 | 21,646,556 | 21,646,561 | 21,646,575 | 21,649,357 |
| 21,685,678 | 21,732,577 | 21,732,578 | 21,732,579 | 21,737,067 | 21,756,111 | 21,840,658 | 21,853,879 | 21,853,881 |
| 21,853,883 | 21,863,617 | 21,863,626 | 21,863,638 | 21,863,662 | 21,881,458 | 21,881,691 | 21,881,814 | 21,889,414 |
| 21,893,958 | 21,900,090 | 21,902,214 | 21,903,750 | 21,903,777 | 21,924,639 | 21,924,665 | 21,953,576 | 21,964,863 |
| 21,972,194 | 21,972,202 | 21,982,758 | 21,987,541 | 21,989,411 | 21,993,039 | 21,993,041 | 21,993,046 | 21,996,680 |
| 21,996,687 | 21,996,691 | 21,996,700 | 21,996,702 | 21,996,706 | 21,996,719 | 22,005,423 | 22,005,428 | 22,005,432 |
| 22,020,061 | 22,020,062 | 22,020,065 | 22,024,009 | 22,035,134 | 22,053,046 | 22,053,047 | 22,056,400 | 22,056,405 |
| 22,058,812 | 22,073,265 | 22,116,673 | 22,119,339 | 22,119,529 | 22,129,262 | 22,131,377 | 22,168,435 | 22,227,347 |
| 22,228,659 | 22,228,668 | 22,228,669 | 22,229,092 | 22,234,749 | 22,239,852 | 22,245,544 | 22,257,403 | 22,257,408 |
| 22,257,416 | 22,257,444 | 22,266,542 | 22,311,743 | 22,327,955 | 22,342,067 | 22,342,072 | 22,342,089 | 22,342,093 |
| 22,342,111 | 22,342,138 | 22,342,145 | 22,342,158 | 22,342,172 | 22,342,180 | 22,342,188 | 22,342,193 | 22,342,196 |
| 22,342,197 | 22,342,212 | 22,342,213 | 22,342,223 | 22,342,226 | 22,342,228 | 22,342,235 | 22,342,237 | 22,342,243 |
| 22,342,246 | 22,342,251 | 22,342,260 | 22,342,268 | 22,342,271 | 22,348,440 | 22,348,441 | 22,373,310 | 22,382,064 |
| 22,395,621 | 22,4i6,288 | 22,476,867 | 22,476,868 | 22,476,369 | 22,476,870 | 22,476,873 | 22,476,877 | 22,559,636 |
| 22,593,186 | 22,593,461 | 22,597,237 | 22,617,368 | 22,619,535 | 22,619,533 | 22,619,540 | 22,619,543 | 22,619,546 |
| 22,619,548 | 22,619,550 | 22,619,556 | 22,619,560 | 22,619,562 | 22,619,563 | 22,619,565 | 22,619,563 | 22,619,570 |
| 22,619,572 | 22, 619,575 | 22,619,577 | 22,619,579 | 22,619,583 | 22,619,586 | 22,619,595 | 22,619,597 | 22,619,599 |
| 22,619,601 | 22,619,605 | 22,619,610 | 22,622,810 | 22,667,472 | 22,667,473 | 22,677,101 | 22,677,105 | 22,677,107 |
| 22,712,891 | 22,718,954 | 22,721,302 | 22,724,594 | 22,726,239 | 22,741,572 | 22,741,573 | 22,741,582 | 22,836,337 |
| 22,924,281 | 22,924,283 | 22,930,289 | 22,944,824 | 22,987,297 | 22,988,973 | 22,988,978 | 22,988,989 | 22,988,998 |
| 22,989,008 | 22,989,019 | 22,989,021 | 22,989,034 | 22,989,043 | 22,939,045 | 22,939,047 | 22,996,790 | 22,996,913 |
| 22,996,914 | 22,996,922 | 22,998,772 | 23,034,978 | 23,035,001 | 23,069,406 | 23,069,577 | 23,106,338 | 23,132,928 |
| 23,132,939 | 23,132,954 | 23,132,960 | 23,132,976 | 23,132,994 | 23,133,066 | 23,133,035 | 23,133,104 | 23,133,103 |
| 23,133,111 | 23,133,135 | 23,133,208 | 23,133,220 | 23,133,233 | 23,133,240 | 23,133,243 | 23,158,323 | 23,172,887 |
| 23,173,756 | 23,234,545 | 23,237,442 | 23,237,443 | 23,237,638 | 23,237,639 | 23,237,640 | 23,263,168 | 23,290,347 |
| 23,290,357 | 23,290,626 | 23,297,456 | 23,297,457 | 23,297,458 | 23,297,460 | 23,297,461 | 23,349,105 | 23,358,537 |
| 23,364,976 | 23,368,821 | 23,434,970 | 23,447,009 | 23,452,070 | 23,452,071 | 23,496,438 | 23,505,292 | 23,622,502 |
| 23,622,503 | 23,626,513 | 23,626,673 | 23,626,677 | 23,626,679 | 23,626,680 | 23,626,842 | 23,626,844 | 23,627,000 |
| 23,627,001 | 23,627,004 | 23,627,005 | 23,627,157 | 23,627,158 | 23,630,531 | 23,633,149 | 23,638,203 | 23,638,567 |
| 23,659,334 | 23,665,040 | 23,667,254 | 23,667,270 | 23,667,271 | 23,669,627 | 23,677,988 | 23,678,227 | 23,686,987 |
| 23,686,988 | 23,686,989 | 23,695,002 | 23,700,084 | 23,715,661 | 23,716,568 | 23,716,909 | 23,719,542 | 23,719,543 |
| 24,182,112 | 24,182,113 | 24,182,140 | 24,182,326 | 24,182,329 | 24,182,333 | 24,182,335 | 24,182,336 | 24,182,337 |
| 24,182,338 | 24,182,339 | 24,182,340 | 24,182,341 | 24,182,342 | 24,182,343 | 24,183,194 | 24,204,782 | 24,204,783 |
| 24,204,784 | 24,204,785 | 24,741,041 | 24,756,103 | 24,798,913 | 24,809,138 | 24,867,560 | 24,884,187 | 24,906,211 |
| 25,022,171 | 25,076,497 | 25,076,666 | 25,193,242 | 25,194,794 | 25,194,796 | 25,194,798 | 25,228,294 | 42,643,355 |
| 44,148,022 | 44,153,142 | 44,153,290 | 44,228,599 | 44,233,597 | 44,238,265 | 44,238,631 | 44,239,672 | 44,240,267 |
| 44,249,223 | 44,512,541 | 44,512,542 | 44,517,919 | 44,517,925 | 44,541,942 | 44,597,116 | 44,717,411 | 44,717,411 |
| 44,717,411 | 44,717,573 | 44,817,506 | 44,887,160 | 45,044,973 | 45,045,138 | 45,045,192 | 45,045,193 | 45,045,194 |
| 43,045,196 | 45,045,199 | 45,045,200 | 45,045,210 | 45,045,211 | 45,049,019 | 45,049,600 | 45,050,451 | 45,050,453 |
| 45,357,504 | 45,480,132 | 45,933,604 | 45,933,659 | 46,183,771 | 46,188,363 | 46,189,690 | 46,237,239 | 46,872,310 |
| 45,899,649 | 46,928,906 | 46,928,909 | 46,929,660 | 46,529,662 | 46,929,564 | 46,929,767 | 46,929,769 | 45,929,771 |
| 46,929,773 | 46,929,775 | 46,923,877 | 46,929,879 | 46,929,881 | 46,929,883 | 46,929, 885 | 46,929,983 | 46,929,935 |
| 46,929,987 | 46,929,989 | 46,929,991 | 46,930,089 | 46,930,738 | 46,930,740 | 46,930,742 | 46,930,744 | 46,930,832 |
| 46,930,834 | 46,930,836 | 46,930,838 | 46,930,840 | 46,930,842 | 46,930,931 | 46,930,933 | 46,930,335 | 49,793,478 |
| 49,836,322 | 49,853,493 | 49,874,343 | \|49,874,344 | 49,874,345 | 49,874,346 | 50,905,225 | 50,905,228 | 50,905,230 |
| 50,907,557 | 50,907,559 | 50,907,773 | 50,907,775 | 50,907,777 | 50,907,779 | 50,908,230 | 50,908,463 | 50,908,465 |
| 50,908,469 | 50,908,470 | 50,911,248 | 50,911,456 | 50,933,198 | 50,934,395 | 50,942,467 | 50,942,549 | 51,031,263 |
| 51,050,603 | 52,951,663 | 52,952,662 | 53,297,348 | 53,443,043 | 53,465,423 | 53,469,680 | 53,471,861 | 54,599,893 |
| 54,600,651 | 54,602,403 | 54,605,141 | 54,605,327 | 54,607,458 | 54,607,453 | 54,607,892 | 54,609,460 | 54,609,451 |
| 54,690,107 | 54,690,110 | 54,690,112 | 56,603,832 | 56,641,575 | 56,642,813 | 56,642,814 | 56,642,815 | 56,649,287 |
| 56,649,288 | 56,649,289 | 56,649,290 | 56,649,291 | 56,649,292 | 56,649,293 | 56,650,053 | 56,838,736 | 56,842,480 |
| 56,927,674 | 56,931,004 | 56,954,063 | 57,347,420 | 57,348,099 | 57,348,139 | 57,348,242 | 57,350,786 | 57,353,046 |
| 57,369,581 | 57,369,582 | 57,370,240 | 57,371,214 | 57,404,131 | 57,404,132 | 57,404,141 | 57,404,144 | 57,479,380 |
| 57,479,381 | 57,801,122 | 57,801,123 | 58,271,550 | 58,271,568 | 58,271,571 | 58,708,617 | 58,954,727 | 58,981,248 |
| 59,423,141 | 59,469,260 | 59,862,225 | 59,876,870 | 59,941,562 | 59,945,033 | 59,945,049 | 59,953,892 | 60,153,069 |
| 60,203,006 | 70,682,647 | 71,295,992 | 71,300,790 | 71,301,085 | 71,301,086 | 71,301,087 | 71,301,088 | 71,301,100 |
| 71,301,433 | 71,306,778 | 71,306,778 | 71,306,979 | 71,311,282 | 71,312,650 | 71,340,449 | 71,345,114 | 71,361,359 |
| 71,372,842 | 71,430,991 | 71,434, 327 | 71,434,328 | 71,447,128 | 71,475,865 | 71,479,473 | 71,500,253 | 71,517,948 |
| 71,528,218 | 71,578,862 | 71,658,082 | 71,732,632 | 71,732,634 | 71,741,547 | 72,164,734 | 72,203,693 | 72,736,046 |
| 72,736,768 | 72,941,503 | 73,212,144 | 73,307,636 | 73,555,299 | 73,555,452 | 73,555,495 | 73,556,093 | 73,557,134 |
| 73,780,045 | 73,894,307 | 73,894,317 | 73,894,318 | 73,394,319 | 74,082,032 | 75,412,563 | 76,963,945 | 77,620,826 |
| 84,819,435 | 85,524,452 | 85,770,041 | 85,787,724 | 85,396,371 | 85,896,374 | 85,972,541 | 86,154,368 | 86,600,076 |
| 86,600,078 | 86,600,080 | 86,629,112 | 86,638,468 | 86,633,469 | 36,658,470 | 86,642,991 | 86,664,723 | 86,738,015 |
| 86,745,982 | 86,745,983 | 36,745,984 | 86,748,578 | 86,748,579 | 86,755,560 | 87,109,675 | 87,148,513 | 87,261,616 |
| 87,261,618 | 87,261,627 | 87,261,632 | 87,261,634 | 87,261,636 | 87,261,637 | 87,261,659 | 87,261,710 | 87,261,723 |
| 87,261,725 | 87,261,733 | 87,261,734 | 37,261,733 | 87,261,740 | 87,261,750 | 87,261,751 | 87,261,753 | 87,261,754 |
| 87,261,758 | 87,273,635 | 87,315,251 | 37,315,420 | 87,315,759 | 87,357,341 | 87,357,342 | 87,372,967 | 87,439,741 |
| 87,475,080 | 87,475,225 | 87,477,991 | 87,574,556 | 87,574,667 | 87,575,532 | 87,575,818 | 87,575,929 | 87,625,935 |
| 87,684,551 | 87,634,552 | 87,702,250 | 87,740,953 | 87,743,651 | 87,386,395 | 87,930,618 | 87,932,050 | 87,950,566 |
| 87,973,266 | 88,095,364 | 88,096,059 | 88,104,535 | 88,157,706 | 88,176,249 | 88,193,568 | 88,222,470 | 88,254,467 |
| 88,258,496 | 88,296,023 | 88,374,845 | 88,413,526 | 88,435,151 | 88,445,516 | 88,445,518 | 88,473,380 | 83,473,411 |
| 88,473,438 | 88,473,759 | 88,492,068 | 88,492,070 | 88,499,769 | 88,607,097 | 88,619,303 | 88,622,678 | 88,624,465 |
| 88,624,466 | 88,626,735 | 88,633.060 | 88,643,234 | 88,689,628 | 88,701,486 | 88,735,730 | 88,738,422 | 88,747,108 |
| 88,749,053 | 88,749,265 | 88,749,279 | 88,749,282 | 88,760,630 | 88,760,633 | 88,760,930 | 88,775,758 | 88,304,969 |
| 90,471,465 | 90,472,492 | 90,472,811 | 90,473,364 | 90,473,686 | 90,476,658 | 90,476,658 | 90,477,046 | 90,478,732 |
| 90,659,536 | 90,661,669 | 91,654,628 | 91,666,506 | 91,666,539 | 91,668,039 | 91,668,040 | 31,867,184 | 91,868,044 |
| 91,868,424 | 91,836,666 | 91,979,898 | 91,996,051 | 91,996,299 | 91,997,848 | 92,004,796 | 92,026,277 | 92,028,425 |
| 92,028,431 | 92,029,717 | 92,043,174 | 102,600,862 | 102,601,227 | 102,601,620 | 102,602,550 | 117,064,703 | 117,064,732 |
| 117,064,750 | 117,064,773 | 117,064,814 | 117,064,943 | 117,065,015 | 117,065,049 | 117,065,281 | 117,065,286 | 117,065,352 |
| 117,065,353 | 117,094,805 | 118,855,762 | 118,856,320 | 119,025,724 | 119,075,307 | 119,075,410 | 119,075,411 | 119,075,412 |
| 119,075,413 | 113,075,414 | l21,225,415 | 121,233,523 | 121,235,197 | 121,513,981 | 122,129,633 | 122,129,634 | 122,404,843 |
| 122,409,375 | 124,219,876 | 127,262,626 | 127,262,627 | 129,627,836 | 129,627,337 | 129,628,445 | 129,631,374 | 129,631,549 |
| 129,631,550 | 129,632,304 | 129,636,318 | 129,637,112 | 129,637,113 | 129,640,894 | 129,655,635 | 129,655,962 | 129,656,034 |
| 129,656,045 | 129,656,052 | 129,663,025 | 129,663,276 | 129,664,704 | 129,664,993 | 129,666,672 | 129,666,688 | 129,665,712 |
| 129,666,726 | 129,672,591 | 129,672,604 | 129,672,603 | 129,672,616 | 129,677,390 | 129,677,407 | 129,673,204 | 129,681,485 |
| 129,681,498 | 129,681,530 | 129,684,391 | 129,684,392 | 129,691,988 | 129,692,842 | 129,708,928 | 129,723,561 | 129,738,338 |
| 129,741,653 | 123,760,359 | 129,762,840 | 129,772,466 | 129,784,023 | 129,794,466 | 129,801,784 | 129,801,784 | 129,801,784 |
| 129,809,167 | 129,809,204 | 129,809,234 | 129,809,241 | 129,809,273 | 129,809,286 | 129,809,383 | 129,815,113 | 129,815,572 |
| 129,815,618 | 129,815,644 | 129,815,671 | 129,816,899 | 129,821,553 | 129,821,554 | 129,826,505 | 129,827,852 | 129,851,725 |
| 129,852,375 | 129,875,229 | 129,885,650 | 129,890,416 | 129,893,591 | 129,893,718 | 129,896,656 | 131,635,452 | 131,697,748 |
| 131,707,326 | 131,707,326 | 131,712,430 | 131,713,084 | 131,719,336 | 131,731,322 | 131,738,151 | 131,739,790 | 131,739,792 |
| 131,842,086 | 131,842,288 | 131,845,348 | 131,870,833 | 131,873,231 | 131,876,773 | 131,878,506 | 131,380,760 | 131,883,481 |
| 131,383,482 | 131,883,905 | 131,883,906 | 131,834,069 | 131,834,369 | 131,884,370 | | | |

TABLE 7
1.28 Exemplary PANDA Agents with structural and transcriptional
activity rescue verified by our experiments. Compounds were randomly selected
from Table 1-Table 6, together with other compounds having only one or two
cysteine-binding potential and experimentally tested their ability in folding p53-
R175H and transcriptionally activating p53-R175H on PUMA promoter using the
PAb1620 IP assay and luciferase reporter assay, respectively. Increasing ‘+’
represents increasing transcriptional activity of p53-R175H on PUMA promoter
upon compound treatment.

| | | | | | Degree of |
|---|---|---|---|---|---|
| | | NSC No. | | Degree | mp53 |
| | | or | | of mp53 | transcriptional |
| | | Sigma | | structural | activity |
| Formula | Structure | Cat No. | CID No. | rescue | rescue |
| KAsO₂ | 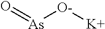 | NSC3060 | 23,668,346 | + | +++++ |
| AsCl₃ | 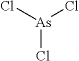 | Sigma 200077 | 24,570 | + | +++++ |
| HAsNa₂O₄ | 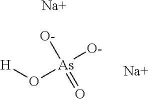 | Sigma A6756 | 61,460 | + | +++++ |
| NaAsO₂ | 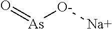 | Sigma S7400 | 443,495 | + | +++++ |
| AsI₃ | 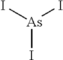 | Sigma 401145 | 24,575 | + | +++++ |
| As₂O₃ | 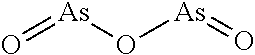 | Sigma 202673 | 261,004 | + | +++++ |
| As₂O₅ | 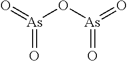 | Sigma 483257 | 14,771 | + | +++++ |
| KAsF₆ | 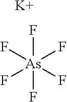 | Sigma 342246 | 159,810 | + | +++ |
| LiAsF₆ | 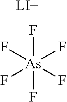 | Sigma 308315 | 9,837,036 | + | +++ |
| SbCl₃ | 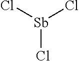 | Sigma 337374 | 24,814 | + | +++ |
| SbF₃ | 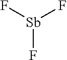 | Sigma 381292 | 24,554 | + | ++ |
| SbAc₃ | 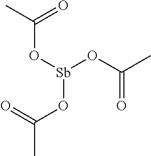 | Sigma 483265 | 16,685,080 | + | ++ |
| Sb₂O₃ | 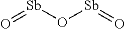 | Sigma 202649 | 27,652 | + | +++ |
| Sb(OC₂H₅)₃ | 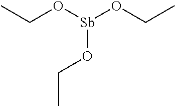 | Sigma 213314 | 16,686,007 | + | +++ |
| Sb(OCH₃)₃ | 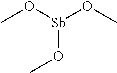 | Sigma 538345 | 16,684,878 | + | +++ |
| SbI₃ | 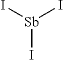 | Sigma 401188 | 14,813 | + | +++ |
| Sb₂O₅ | 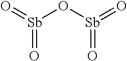 | Sigma 255998 | 14,813 | + | +++ |
| Sb₂(SO₄)₃ | 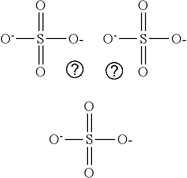 [SMILES: O=S(=O)([O-])[O-] ; O=S(=O)([O-])[O-] ; O=S(=O)([O-])[O-]] | Sigma 10783 | 24,010 | + | +++ |
| BiI₃ | 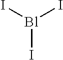 | Sigma 229474 | 111,042 | + | +++ |
| C₁₆H₁₈As₂N₄O₂ | 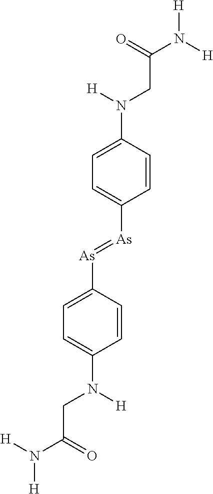 | NSC92909 | 261,046 | + | + |
| C₁₃H₁₄As₂O₆ | 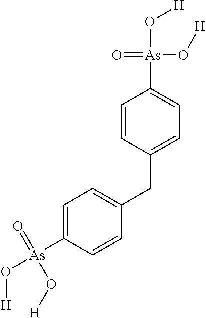 | NSC48300 | 241,158 | + | +++ |
| C₁₇H₂₈AsClN₄O₆S | 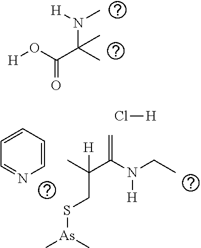 | NSC721951 | 405,069 | + | + |
| C₁₀H₁₃NO₈Sb | 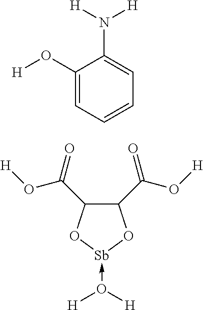 | NSC31660 | 16,682,749 | + | +++ |
| C₆H₁₂NaO₈Sb+ | 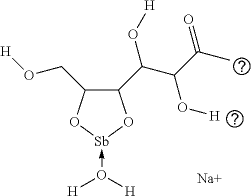 | NSC15609 | 24,182,331 | + | +++ |
| (CH₃CO₂)₃Sb | 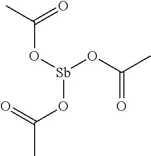 | Sigma 483265 | 16,685,080 | + | + |
| C₈H₄K₂O₁₂Sb₂•xH₂O | 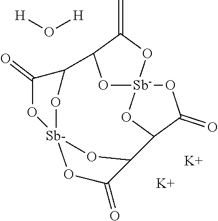 | Sigma 244791 | 53,315,432 | + | ++ |
| C₁₃H₂₁NaO₉Sb+ | 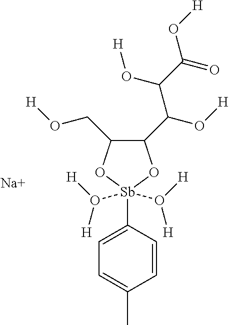 | NSC15623 | 24,182,342 | + | +++ |
| HOC₆H₄COOBiO | 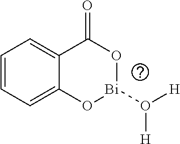 | Sigma 480789 | 16,682,734 | + | + |
| (O₂CCh₂C(OH)(CO₂)CH₂CO₂]Bi | 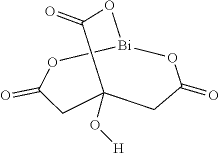 | Sigma 480746 | 16,696,198 | + | +++ |
| (CH₃CO₂)₃Bi | 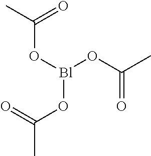 | Sigma 401587 | 16,688,082 | + | +++ |
| As₂S₂ | 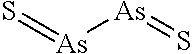 | Sigma 519111 | 3,627,253 | + | +++++ |
| As₂S₃ | 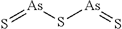 | Sigma 448060 | 4,093,503 | + | +++++ |
| As₂S₅ | 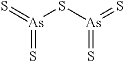 | Sigma 519103 | 3,371,533 | + | +++++ |

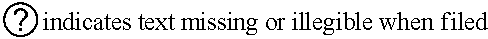

TABLE 8
Rescue profile of selected mp53. Str. Res. column shows whether
the mp53 is structurally rescuable. Func. Res. shows whether the mp53 is
functionally rescuable. Res. column shows whether the mp53 is rescuable (i.e.
either structurally or functionally rescuable). Mutations are selected from
clinical p53 mutations detected by Shanghai Institute of Hematology (SIH) and
p53 mutations reported in MDS patients (FIG. 4), and our clinical data.

| | Str. | Func. | | | Str. | Func. | | | Str. | Func. | |
|---|---|---|---|---|---|---|---|---|---|---|---|
| mp53 | Res. | Res. | Res. | mp53 | Res. | Res. | Res. | mp53 | Res. | Res. | Res. |
| R17H | Yes | Yes | Yes | K132M | Yes | Yes | Yes | D261H | Yes | Yes | Yes |
| R245S | Yes | Yes | Yes | A138V | No | Yes | Yes | D281Y | Yes | No | Yes |
| R248Q | Yes | Yes | Yes | G154S | Yes | No | Yes | R283H | Yes | Yes | Yes |
| R249S | Yes | Yes | Yes | R156P | No | Yes | Yes | L383P | No | No | No |
| R273H | No | No | No | A159V | Yes | Yes | Yes | M384T | No | No | No |
| R282W | Yes | Yes | Yes | A159P | Yes | Yes | Yes | F054Y | Yes | Yes | Yes |
| T232T | Yes | Yes | Yes | M160L | No | No | Yes | S090P | Yes | Yes | Yes |
| F270C | No | Yes | Yes | Y163H | Yes | Yes | Yes | Q375X | Yes | Yes | 'es |
| Y220H | No | Yes | Yes | Y163C | Yes | Yes | Yes | Q038H | Yes | Yes | Yes |
| I254T | Yes | Yes | Yes | R174L | Yes | Yes | Yes | S241A | No | Yes | Yes |
| P273C | No | No | No | C176Y | Yes | Yes | Yes | S241C | Yes | Yes | Yes |
| C176F | Yes | No | Yes | H179Y | Yes | Yes | Yes | S241D | Yes | Yes | Yes |
| H179P | Yes | Yes | Yes | H179Q | Yes | Yes | Yes | S241E | Yes | Yes | Yes |
| Y220C | Yes | No | No | P190L | Yes | Yes | Yes | S241F | Yes | Yes | Yes |
| R278S | No | No | No | H193R | Yes | Yes | Yes | S241G | No | Yes | Yes |
| Vl43A | Yes | Yes | Yes | R20'9K | Yes | No | Yes | S241H | No | Yes | Yes |
| S006P | No | No | No | V216E | No | Yes | Yes | S241E | Yes | Yes | Yes |
| L014P | No | No | No | Y234H | Yes | Yes | Yes | S241K | No | No | No |
| S033P | No | Yes | Yes | M23IT | Yes | Yes | Yes | S241L | No | Yes | Yes |
| Q052R | No | No | No | V272N | Yes | Yes | Yes | S241M | Yes | Yes | Yes |
| D057G | No | Yes | Yes | C238Y | Yes | Yes | Yes | S241N | Yes | Yes | Yes |
| D061G | No | Yes | Yes | G245A | Yes | Yes | Yes | S241P | Yes | No | Yes |
| P072A | No | No | No | G245D | Yes | Yes | Yes | S241Q | Yes | Yes | Yes |
| P080S | No | No | No | R248W | No | Yes | Yes | S241R | Yes | Yes | Yes |
| T081P | No | No | No | G266R | No | Yes | Yes | S241T | No | Yes | Yes |
| S094P | No | No | No | F270S | No | Yes | Yes | S241V | Yes | Yes | Yes |
| S095F | No | No | No | R273S | No | No | No | S241W | Yes | Yes | Yes |
| Y126C | No | Yes | Yes | R273L | No | No | No | S241Y | Yes | No | Yes |
| L130H | Yes | Yes | Yes | R278H | No | No | No | | | | |

TABLE 9
1.30 Representative mp53 rescuability experimental data. Structural rescuability for the indicated mp53 was measured by comparing the PAb1620
immunoprecipitation efficiency of the mp53 in the presence and absence of the PANDA Agent ATO. Functional rescuability for the indicated mp53
was measured by the functional assays Luciferase, qPCR, and/or Western blot for the indicated mp53 target genes in the presence and absence
of the PANDA Agent ATO. A p53 mutation is rescuable if it is neither functionally nor structurally rescuable. A p53 mutation is non-rescuable if it
is neither functionally nor structurally rescuable. Other PANDA Agents also produced a similar rescuability profile.

| | 1620 | | | | | | | | | | | | | | | | |
|---|---|---|---|---|---|---|---|---|---|---|---|---|---|---|---|---|---|
| | TP | Luciferase Assay | | | | | | | | | | qPCR | | | | | |
| | / | CDKNIA | | | PUMA | | | MDM2 | | | PIG3 | CDKNIA | PUMA | MDM2 | Str. | Func. | |
| | H | H | HC | S2 | H | HC | S2 | H | HC | S2 | H | H | H | H | Res | Res | |
| R175H | 39.6 | 3.8 | 1.4 | 1.5 | 6.2 | 1.0 | 1.5 | 8.1 | 2.4 | 1.6 | | 6.8 | 2.2 | 3.3 | Yes | Yes | |
| R245S | 1.6 | 3.7 | 2.6 | 2.6 | 2.1 | 1.7 | 1.1 | 2.7 | 3.3 | 4.2 | 2.3 | | | | Yes | Yes | |
| R248Q | 2.1 | 1.3 | 1.3 | 1.3 | 1.1 | 1.0 | 1.3 | 1.4 | 2.1 | 1.3 | 0.9 | | | | Yes | Yes | |
| R249S | 19.4 | 3.9 | 1.2 | 1.5 | 1.1 | 0.9 | 1.3 | 1.7 | 2.6 | 1.2 | | | | | Yes | Yes | |
| R273H | 1.4 | 1.3 | 1.1 | 1.4 | 1.1 | 1.0 | 1.4 | 1.5 | 1.4 | 1.3 | 1.1 | | | | No | No | |
| R282W | 3.5 | 6.8 | 5.2 | 9.9 | 17.0 | 4.1 | 13.1 | 5.1 | 3.0 | 3.2 | 6.5 | | | | Yes | Yes | |
| I232T | 1.6 | 6.2 | 4.6 | 6.0 | 7.7 | 3.6 | 4.0 | 2.4 | 2.7 | 3.2 | 4.3 | | | | Yes | Yes | |
| F270C | 1.1 | 8.0 | 3.1 | 3.8 | 8.1 | 2.5 | 3.2 | 1.4 | 2.1 | 1.9 | 2.1 | | | | No | Yes | |
| Y220H | 1.0 | | | | 4.4 | | | | | | | 14.5 | 5.1 | 2.1 | No | Yes | |
| I254T | 1.8 | | | | 7.8 | | | | | | | 3.2 | 3.4 | 2.9 | Yes | Yes | |
| R273C | 1.2 | | | | 1.0 | | | | | | | 0.9 | 1.3 | 0.6 | No | No | |
| | 1620 | | | | | | 1620 | | | | | | 1620 | | | | |
| | TP | Luciferase Assayu | | STR. | Func. | | TP | Luciferase Assay | | Str. | Func. | | TP | Western Bloat | | Str. | Func. |
| | / | CDKNIA | PUMA | Res | RES | | / | CDKNIA | PUMA | Res. | Res. | | / | Puma | p21 | Res | Res. |
| C176F | 2.7 | 1.2 | | Yes | No | G245A | 3.2 | | 3.2 | Yes | Yes | S241A | 1.47 | 2.2 | 1.0 | No | Yes |
| H179R | 4.6 | 1.0 | 1.8 | Yes | Yes | G2450 | 1.7 | | 1.5 | Yes | Yes | | | | | | |
| Y220C | 3.9 | 1.4 | 1.0 | Yes | No | R248W | 1.1 | | 1.5 | No | Yes | | | | | | |
| R278S | 1.0 | 1.1 | | No | No | G266R | 1.0 | | 3.2 | No | Yes | | | | | | |
| V143A | 6.5 | 2.5 | | Yes | Yes | F270S | 1.0 | | 1.9 | No | Yes | | | | | | |
| S006P | 1.2 | | 1.3 | No | No | R273S | 1.49 | | 1.0 | No | No | | | | | | |
| L014P | 1.2 | | 1.1 | No | No | R273L | 1.2 | | 1.0 | No | No | | | | | | |
| S033P | 1.0 | | 1.9 | No | Yes | P278H | 1.1 | | 1.0 | No | No | | | | | | |
| Q052R | 1.1 | | 1.4 | No | No | D281H | 2.1 | | 2.2 | Yes | Yes | | | | | | |
| D057G | 1.0 | | 1.5 | No | Yes | D281Y | 1.5 | | 1.1 | Yes | No | | | | | | |
| D061G | 1.1 | | 2.1 | No | Yes | R283H | 1.6 | | 1.6 | Yes | Yes | | | | | | |
| P072A | 0.9 | | 1.3 | No | No | L383P | 1.1 | | 1.2 | No | No | | | | | | |
| P080S | 1.0 | | 1.0 | No | No | M384T | 1.2 | | 1.0 | No | No | | | | | | |
| T081P | 1.49 | | 1.2 | No | No | F054Y | 1.6 | | 1.2 | Yes | Yes | | | | | | |
| S094P | 0.9 | | 0.9 | No | No | S090P | 1.6 | | 1.2 | Yes | Yes | | | | | | |
| S095F | 1.1 | | 1.2 | No | No | Q375X | 2.0 | | 1.3 | Yes | Yes | | | | | | |
| Y126C | 0.9 | | 2.9 | No | Yes | Q038H | 1.7 | | 1.5 | Yes | Yes | | | | | | |
| L130H | 86.7 | | 9.5 | Yes | Yes | | | | | | | | | | | | |
| K132M | 1.6 | | 2.5 | Yes | Yes | | | | | | | | | | | | |
| A138V | 1.1 | | 4.8 | No | Yes | | | | | | | | | | | | |
| G154S | 2.1 | | 1.2 | Yes | No | | | | | | | | | | | | |
| | 1620 | | | | | | | | 1620 | | | | | | | | |
| | TP | Luciferase Assay | | | | STR. | Runc | | TP | Luciferase Assay | | | | Str. | Func. | | |
| | / | CDKNIA | | PUMA | | Res | Res | | / | CDknia | | PUMA | | Res. | Res. | | |
| R156P | 1.0 | | | 3.3 | | No | Yes | S241C | 2.7 | 1.1 | | 1.8 | | Yes | Yes | | |
| A159V | 2.9 | | | 7.8 | | Yes | Yes | S241D | 2.1 | 2.7 | | 1.4 | | Yes | Yes | | |
| A159P | 2.8 | | | 2.8 | | Yes | Yes | S241E | 1.7 | 0.9 | | 1.8 | | Yes | Yes | | |
| M160L | 0.0 | | | 1.1 | | No | No | S241F | 1.9 | 0.6 | | 2.2 | | Yes | Yes | | |
| Y163H | 6.5 | | | 3.0 | | Yes | Yes | S241G | 1.4 | 1.1 | | 1.9 | | No | Yes | | |
| Y163C | 10.4 | | | 3.1 | | Yes | Yes | S241H | 1.1 | 2.1 | | 1.1 | | No | Yes | | |
| R174L | 2.8 | | | 2.2 | | Yes | Yes | S241I | 1.6 | 1.6 | | 2.8 | | Yes | Yes | | |
| C176Y | 26.1 | | | 2.8 | | Yes | Yes | S241K | 1.3 | 1.4 | | 0.9 | | No | No | | |
| H179Y | 78.7 | | | 2.1 | | Yes | Yes | S241L | 1.4 | 2.9 | | 1.5 | | No | Yes | | |
| H179Q | 2.3 | | | 1.8 | | Yes | Yes | S241M | 1.5 | 0.8 | | 1.8 | | Yes | Yes | | |
| P190L | 2.7 | | | 4.3 | | Yes | Yes | S241N | 2.7 | 8.9 | | 1.5 | | Yes | Yes | | |
| H193R | 2.4 | | | 2.0 | | Yes | Yes | S241P | 4.2 | 0.6 | | 1.0 | | Yes | No | | |
| R209K | 2.0 | | | 1.4 | | Yes | No | S241Q | 2.0 | 1.7 | | 7.4 | | Yes | Yes | | |
| V216E | 1.0 | | | 2.3 | | No | Yes | S241R | 2.3 | 11.0 | | 1.6 | | Yes | Yes | | |
| Y234H | 2.7 | | | 48.9 | | Yes | Yes | S241T | 1.1 | 1.9 | | 1.4 | | No | Yes | | |
| M237I | 82.4 | | | 2.4 | | Yes | Yes | S241V | 2.8 | 0.4 | | 2.5 | | Yes | Yes | | |
| V272M | 4.3 | | | 28.4 | | Yes | Yes | S241W | 1.7 | 5.7 | | 2.1 | | Yes | Yes | | |
| C238Y | 68.3 | | | 1.6 | | Yes | Yes | S241Y | 1.8 | 1.1 | | 1.3 | | Yes | No | | |

Str. Res.: one or more of the wildtype structure in the indicated mp53 can be rescured by the PANDA Agent ATO, as measured to a 1.5 fold or more increase in PAb1620 immunoprecipitation signal.
A “Yes” in this column supports the presence of a structurally rescuable p53 mutation.
Fun. Res.: one or more of the mutation is a functionally rescuable p53 mutation, as indicated by a “Yes” under the colun. In particular, the PANDA Agent can rescue one or more of the mp53's wild type
function to a 1.5 fold or more, as measured by luciferase assay, qPCR, or Western blot.
The cell expressing the mp53s include H1299 non-small cell lung carcinoma cell (“H”), HC T116 colon cancer cells (“HC”), and Saos-2 osteosarcoma (“S2”).

TABLE 10
Patient selection criteria for our phase I Decitabine (“DAC”) -
ATO combination therapy trial for Myelodysplastic Syndrome
(DMS). Patients with mutant TP53
tested for rescuability, and those with rescuable mp53 are
selected for trial.
Characteristics of the 50 sequenced MDS/AML patients at diagnosis

| | All | Mutant | Wild-Type |
|---|---|---|---|
| | Patients | TP53 | TP53 |
| Characteristic | (N = 50) | (N = 3) | (N = 47) |
| Exome sequencing performed - | 50 (100) | 3 (100) | 47 (100) |
| No. (%) | | | |
| Male sex - No. (%) | 27 (54) | 0 (0) | 27 (57) |
| Age at diagnosis - yrs | | | |
| Median | 57.5 | 62 | 57 |
| Range | 17-92 | 49-64 | 17-82 |
| Disease - No. (%) | | | |
| AML | 5 (10) | 0 (0) | 5 (11) |
| MDS | 45 (90) | 3 (100) | 42 (89) |

TABLE 11
1.32 Treatment response observed in our phase I Decitabine (“DAC”) - ATO combination therapy trial for Myelodysplastic Syndrome (DMS).
Clinical characteristics and responses of the three treated mp53 patients (#27, #35, and #19)

| | | | | | Hb; | | | | | |
|---|---|---|---|---|---|---|---|---|---|---|
| | | | Diag.; | BM | Platelets; | | | | Study | Best |
| Patient | Sex | mp53 | Age (yr) | Blast | ANC | Karyotype | IPSS-R | Regimen | (days) | Response |
| #27 | F | S241F | MDS- | 7.5% | 70 g/L; | 46XX, | High | DAC + | 259 | Complete |
| | | | RAEB | | 101 × 10⁹/L; | 6p+ | risk | ATO | | Remission |
| | | | (62) | | 1.85 × 10⁹/L | | | | | |
| #35 | F | S241C | MDS- | 10.5% | 63 g/L; | 46XX | High | DAC + | 359 | Complete |
| | | | RAEB-II | | 20 × 10⁹/L; | | risk | ATO | | Remission |
| | | | (63) | | 0.77 × 10⁹/L | | | | | |
| #19^{a} | F | Q375*, | MDS- | 6.5% | 94 g/L; | 46XXX, +del (3) (p21), −5, −13, | Very | DAC + | 166 | Progression- |
| | | Q38H | RAEB-I | | 34 × 10^{9/}L; | +mar [12]/46, idem, −7, +8, −14, | high | ATO + | | free |
| | | | (62) | | 1.74 × 10⁹/L | −17, −19, +mar2, +mar3, +mar4 | risk | ARA | | |
| | | | | | | [2]/46XX [5] | | | | |

^{a}Patient No. 19 was DAC-resistant and AML -prone
“Hb” means Haemoglobin
*means the mutations causes a stope code on p53 gene sequence, leading to a truncated mp53

TABLE 12
Adverse effects observed in our phase I Decitabine
(“DAC”) - ATO combination therapy trial for
Myelodysplastic Syndrome (DMS).

| Adverse | Patient: | | |
|---|---|---|---|
| events of treatment | #27 | #35 | #19 |
| WBC (1 × 100) | 2 | 2 | 2 |
| PLT (1 × 109) | 0 | 2 | 2 |
| Hb (g/L) | 0 | 0 | 1 |
| Fever/No. infection | 1 | 1 | 1 |
| Weight gain/loss | 0 | 0 | 1 |
| Nausea | 0 | 0 | 1 |
| Vomiting | 0 | 0 | 1 |
| Diarrhea | 0 | 0 | 0 |
| Fatigue | 0 | 0 | 1 |
| Constipation | 1 | 0 | 0 |
| Dyspnead | 0 | 0 | 0 |
| Dysrhythmias | 0 | 0 | 1 |
| Stomatitis | 0 | 0 | 1 |
| SGOT/SGPT | 0 | 0 | 0 |
| Blood creatinine increased | 0 | 0 | 0 |
| Hypokalemia | 0 | 0 | 0 |

TABLE 13
Exemplary p53 SNPs

| | | | | | |
|---|---|---|---|---|---|
| P47S | P72R | V217M | G360A | R110L/P | R267W |
| P278A | R290H | N311S | E339K | S366A | |

TABLE 14
p53 Isoforms, Nomenclatures and Sequences

| | p53 amino acid sequence, |
|---|---|
| p53 Nomenclature and reference | PANDA Cysteines are underlined |
| Wildtype human p53 isoform a | 393aa |
| NCBI Reference Sequence: NP_000537.3 cellular | MEEPQSDPSVEPPLSQETFSDLWKLLPENNVLSPLP |
| tumor antigen p53 isoform a [Homo sapiens]; | SQAMDDLMLSPDDIEQWFTEDPGPDEAPRMPEAAPP |
| NCBI Reference Sequence: NP_001119584.1, | VAPAPAAPTPAAPAPAPSWPLSSSVPSQKTYQGSYG |
| NP_001119584.1 cellular tumor antigen p53 | FRLGFLHSGTAKSVTCTYSPALNKMFCQLAKTCPVQ |
| isoform a [Homo sapiens] | LWVDSTPPPGTRVRAMAIYKQSQHMTEVVRRCPHHE |
| Other Names: | RCSDSDGLAPPQHLIRVEGNLRVEYLDDRNTFRHSV |
| p53 isoform 1 | VVPYEPPEVGSDCTTIHYNYMCNSSCMGGMNRRPIL |
| UniProt database identifier: P04637-1, | TIITLEDSSGNLLGRNSFEVRVCACPGRDRRTEEEN |
| sp\|P04637\|P53_HUMAN Cellular tumor antigen p53 | LRKKGEPHHELPPGSTKRALPNNTSSSPQPKKKPLD |
| OS = Homo sapiens GN = TP53 PE = 1 SV = 4 | GEYFTLQIRGRERFEMFRELNEALELKDAQAGKEPG |
| p53 | GSRAHSSHLKSKKGQSTSRHKKLMFKTEGPDSD |
| full-length p53 | |
| p53α. | |
| Wildtype human p53 isoform b | 341aa |
| NCBI Reference Sequence: NP_001119586.1, | MEEPQSDPSVEPPLSQETFSDLWKLLPENNVLSPLP |
| NP_001119586.1 cellular tumor antigen p53 isoform b | SQAMDDLMLSPDDIEQWFTEDPGPDEAPRMPEAAPP |
| [Homo sapiens] | VAPAPAAPTPAAPAPAPSWPLSSSVPSQKTYQGSYG |
| Other Names: | FRLGFLHSGTAKSVTCTYSPALNKMFCQLAKTCPVQ |
| p53 isoform 2 | LWVDSTPPPGTRVRAMAIYKQSQHMTEVVRRVPHHE |
| UniProt database identifier: P04637-2, | RCSDSDGLAPPQHLIRVEGNLRVEYLDDRNTFRHSV |
| sp\|04637-2\|P53_HUMAN Isoform 2 of Cellular | VVPYEPPEVGSDCTTIHYNYMCNSSCMGGMNRRPIL |
| tumor antigen p53 OS = Homo sapiens GN = TP53 | TIITLEDSSGNLLGRNSFEVRVCACPGRDRRTEEEN |
| p53β. | LRKKGEPHHELPPGSTKRALPNNTSSSPQPKKKPLD |
| | GEYFTLQDQTSFQKENC |
| Wildtype human p53 isoform c | 346aa |
| NCBI Reference Sequence: NP_001119585.1, | MEEPQSDPSVEPPLSQETFSDLWKLLPENNVLSPLP |
| NP_001119585.1 cellular tumor antigen p53 isoform c | SQAMDDLMLSPDDIEQWFTEDPGPDEAPRMPEAAPP |
| [Homo sapiens] | VAPAPAAPTPAAPAPAPSWPLSSSVPSQKTYQGSYG |
| Other Names: | FRLGFLHSGTAKSVTCTYSPALNKMFCQLAKTCPVQ |
| p53 isoform 3 | LWVDSTPPPGTRVRAMAIYKQSQHMTEVVRRCPHHE |
| UniProt database identifier: P04637-3, | RCSDSDGLAPPQHLIRVEGNLRVEYLDDRNTFRHSV |
| sp\|P04637-3\|P53_HUMAN Isoform 3 of Cellular | VVPYEPPEVGSDCTTIHYNYMCNSSCMGGMNRRPIL |
| tumor antigen p53 OS = Homo sapiens GN= TP53 | TIITLEDSSGNLLGRNSFEVRVCACPGRDRRTEEEN |
| p53γ | LRKKGEPHHELPPGSTKRALPNNTSSSPQPKKKPLD |
| | GEYFTLQMLLDLRWCYFLINSS |
| Wildtype human p53 isoform g | 354aa |
| NCBI Reference Sequence: NP_001119590.1, | MDDLMLSPDDIEQWFTEDPGPDEAPRMPEAAPPVAP |
| NP_001119590.1 cellular tumor antigen p53 isoform g | APAAPTPAAPAPAPSWPLSSSVPSQKTYQGSYGFRL |
| [Homo sapiens]; | GFLHSGTAKSVTCTYSPALNKMFCQLAKTCPVQLWV |
| NCBI Reference Sequence: NP_001263689.1, | DSTPPPGTRVRAMAIYKQSQHMTEVVRRCPHHERCS |
| NP_001263689.1 cellular tumor antigen p53 isoform g | DSDGLAPPQHLIRVEGNLRVEYLDDRNTFRHSVVVP |
| [Homo sapiens]; | YEPPEVGSDCTTIHYNYMCNSSCMGGMNRRPILTII |
| NCBI Reference Sequence: NP_001263690.1, | TLEDSSGNLLGRNSFEVRVCACPGRDRRTEEENLRK |
| NP_001263690.1 cellular tumor antigen p53 isoform g | KGEPHHELPPGSTKRALPNNTSSSPQPKKKPLDGEY |
| [Homo sapiens]) | FTLQIRGRERFEMFRELNEALELKDAQAGKEPGGSR |
| Other Names: | AHSSHLKSKKGQSTSRHKKLMFKTEGPDSD |
| p53 isoform 4 | |
| UniProt database identifier: P04637-4, | |
| sp\|P04637-4\|P53_HUMAN Isoform 4 of Cellular | |
| tumor antigen p53 OS = Homo sapiens GN = TP53) | |
| Δ40p53α | |
| Wildtype human p53 isoform i | 302aa |
| NCBI Reference Sequence: NP_001263625.1, | MDDLMLSPDDIEQWFTEDPGPDEAPRMPEAAPPVAP |
| NP_001263625.1 cellular tumor antigen p53 isoform i | APAAPTPAAPAPAPSWPLSSSVPSQKTYQGSYGFRL |
| [Homo sapiens] | GFLHSGTAKSVTCTYSPALNKMFCQLAKTCPVQLWV |
| Other Names: | DSTPPPGTRVRAMAIYKQSQHMTEVVRRCPHHERCS |
| p53 isoform 5 | DSDGLAPPQHLIRVEGNLRVEYLDDRNTFRHSVVVP |
| UniProt database identifier: P04637-5, | YEPPEVGSDCTTIHYNYMCNSSCMGGMNRRPILTII |
| sp\|P04637-5\|P53_HUMAN Isoform 5 of Cellular | TLEDSSGNLLGRNSFEVRVCACPGRDRRTEEENLRK |
| tumor antigen p53 OS = Homo sapiens GN = TP53) | KGEPHHELPPGSTKRALPNNTSSSPQPKKKPLDGEY |
| Δ40p53β | FTLQDQTSFQKENC |
| Wildtype human p53 isoform h | 307aa |
| NCBI Reference Sequence: NP_001263624.1, | MDDLMLSPDDIEQWFTEDPGPDEAPRMPEAAPPVAP |
| NP_001263624.1 cellular tumor antigen p53 isoform h | APAAPTPAAPAPAPSWPLSSSVPSQKTYQGSYGFRL |
| [Homo sapiens]) | GFLHSGTAKSVTCTYSPALNKMFCQLAKTCPVQLWV |
| Other Names: | DSTPPPGTRVRAMAIYKQSQHMTEVVRRCPHHERCS |
| p53 isoform 6 | DSDGLAPPQHLIRVEGNLRVEYLDDRNTFRHSVVVP |
| UniProt database identifier: P04637-6, | YEPPEVGSDCTTIHYNYMCNSSCMGGMNRRPILTII |
| sp\|P04637-6\|P53_HUMAN Isoform 6 of Cellular | TLEDSSGNLLGRNSFEVRVCACPGRDRRTEEENLRK |
| tumor antigen p53 OS = Homo sapiens GN = TP53 | KGEPHHELPPGSTKRALPNNTSSSPQPKKKPLDGEY |
| Δ40p53γ | FTLQMLLDLRWCYFLINSS |

### 1.36 Representative Effective Dose for Mouse Studies

TABLE 15
Representative effective dose for administering
in mouse.

| | Drug concen- | |
|---|---|---|
| | tration when | |
| | administration | Dose per day |
| ATO (i .v.) | 1 mg/ml | 5 mg/kg |
| ATO (oral) | 1 mg/ml | 5 mg/kg |
| As2S2 (oral) | 15 mg/ml | 100 mg/kg |
| As2S3 (oral | 15 mg/ml | 100 mg/kg |
| As2S5 (oral) | 15 mg/ml | 100 mg/kg |
| As4S4 (oral) | 15 mg/ml | 100 mg/kg |

TABLE 16
Representative effective dose in humans.

| | | | Maximun | Platform |
|---|---|---|---|---|
| | Adminis- | | Metal | Metal |
| | tering | | concen- | concen- |
| | drug | | tration | tration |
| | concen- | Dose | in blood | in blood |
| | tration | per day | (plasma) | (plasma) |
| ATO (i.v.) | 0.02-0.04 | 0.1-0.3 | 0.57-1.31 | 0.03-0.07 |
| | mg/ml | mg/kg | mg/L | mg/L |
| ATO (oral) | 1.25 | 0.13 | 0.57-1.31 | 0.03-0.07 |
| | mg/ml | mg/kg | mg/L | mg/L |
| As2S2 (oral) | Solid | 2.80 | 0.57-1.31 | 0.03-0.07 |
| | | mg/kg | mg/L | mg/L |
| As2S3 (oral) | Solid | 3.20 | 0.57-1.31 | 0.03-0.07 L |
| | | mg/kg | mg/L | mg/ |
| As2S5 (oral) | Solid | 4.05 | 0.57-1.31 | 0.03-0.07 |
| | | mg/kg | mg/L | mg/L |
| As4S4 (oral) | Solid | 2.80 | 0.57-1.31 | 0.03-0.07 |
| | | mg/kg | mg/L | mg/L |

### REFERENCES

The following publications, references, patents and patent applications are hereby incorporated by reference in their entireties.
Alexandrova, E. M., Yallowitz, A. R., Li, D., Xu, S., Schulz, R., Proia, D. A., Lozano, G., Dobbelstein, M., and Moll, U. M. (2015). Improving survival by exploiting tumour dependence on stabilized mutant p53 for treatment. Nature 523, 352-356.
Aryee, D. N., Niedan, S., Ban, J., Schwentner, R., Muehlbacher, K., Kauer, M., Kofler, R., and Kovar, H. (2013). Variability in functional p53 reactivation by PRIMA-1(Met)/APR-246 in Ewing sarcoma. British journal of cancer 109, 2696-2704.
Basse, N., Kaar, J. L., Settanni, G., Joerger, A. C., Rutherford, T. J., and Fersht, A. R. (2010). Toward the rational design of p53-stabilizing drugs: probing the surface of the oncogenic Y220C mutant. Chemistry & biology 17, 46-56.
Bauer, M. R., Joerger, A. C., and Fersht, A. R. (2016). 2-Sulfonylpyrimidines: Mild alkylating agents with anticancer activity toward p53-compromised cells. Proceedings of the National Academy of Sciences of the United States of America 113, E5271-5280.
Boeckler, F. M., Joerger, A. C., Jaggi, G., Rutherford, T. J., Veprintsev, D. B., and Fersht, A. R. (2008). Targeted rescue of a destabilized mutant of p53 by an in silico screened drug. Proceedings of the National Academy of Sciences of the United States of America 105, 10360-10365.
Bullock, A. N, and Fersht, A. R. (2001). Rescuing the function of mutant p53. Nature reviews Cancer 1, 68-76.
Bullock, A. N, Henckel, J., DeDecker, B. S., Johnson, C. M., Nikolova, P. V., Proctor, M. R., Lane, D. P., and Fersht, A. R. (1997). Thermodynamic stability of wild-type and mutant p53 core domain. Proceedings of the National Academy of Sciences of the United States of America 94,14338-14342.
Bullock, A. N, Henckel, J., and Fersht, A. R. (2000). Quantitative analysis of residual folding and DNA binding in mutant p53 core domain: definition of mutant states for rescue in cancer therapy. Oncogene 19, 1245-1256.
Bykov, V. J., Issaeva, N., Shilov, A., Hultcrantz, M., Pugacheva, E., Chumakov, P., Bergman, J., Wiman, K. G., and Selivanova, G. (2002). Restoration of the tumor suppressor function to mutant p53 by a low-molecular-weight compound. Nature medicine 8, 282-288.
Cancer Genome Atlas Research, N., Ley, T. J., Miller, C., Ding, L., Raphael, B. J., Mungall, A. J., Robertson, A., Hoadley, K., Triche, T. J., Jr., Laird, P. W., et al. (2013). Genomic and epigenomic landscapes of adult de novo acute myeloid leukemia. The New England journal of medicine 368, 2059-2074.
Demma, M., Maxwell, E., Ramos, R., Liang, L., Li, C., Hesk, D., Rossman, R., Mallams, A., Doll, R., Liu, M., et al. (2010). SCH529074, a small molecule activator of mutant p53, which binds p53 DNA binding domain (DBD), restores growth-suppressive function to mutant p53 and interrupts HDM2-mediated ubiquitination of wild type p53. The Journal of biological chemistry 285, 10198-10212.
Demma, M. J., Wong, S., Maxwell, E., and Dasmahapatra, B. (2004). CP-31398 restores DNA-binding activity to mutant p53 in vitro but does not affect p53 homologs p63 and p73. The Journal of biological chemistry 279, 45887-45896.
Dolgin, E. (2017). The most popular genes in the human genome. Nature 551, 427-431.
Donoghue, N., Yam, P. T., Jiang, X. M., and Hogg, P. J. (2000). Presence of closely spaced protein thiols on the surface of mammalian cells. Protein science: a publication of the Protein Society 9, 2436-2445.
Feldser, D. M., Kostova, K. K., Winslow, M. M., Taylor, S. E., Cashman, C., Whittaker, C. A., Sanchez-Rivera, F. J., Resnick, R., Bronson, R., Hemann, M. T., et al. (2010). Stage-specific sensitivity to p53 restoration during lung cancer progression. Nature 468, 572-575.
Fogal, V., Hsieh, J. K., Royer, C., Zhong, S., and Lu, X. (2005). Cell cycle-dependent nuclear retention of p53 by E2F1 requires phosphorylation of p53 at Ser315. The EMBO journal 24, 2768-2782.
Foster, B. A., Coffey, H. A., Morin, M. J., and Rastinejad, F. (1999). Pharmacological rescue of mutant p53 conformation and function. Science 286, 2507-2510.
Freed-Pastor, W. A., and Prives, C. (2012). Mutant p53: one name, many proteins. Genes & development 26, 1268-1286.
Gao, J., Aksoy, B. A., Dogrusoz, U., Dresdner, G., Gross, B., Sumer, S. O., Sun, Y., Jacobsen, A., Sinha, R., Larsson, E., et al. Integrative analysis of complex cancer genomics and clinical profiles using the cBioPortal. Sci Signal 6, p11.
Gillotin, S., Yap, D., and Lu, X. (2010). Mutation at Ser392 specifically sensitizes mutant p53H175 to mdm2-mediated degradation. Cell cycle 9, 1390-1398.
Grellety, T., Laroche-Clary, A., Chaire, V., Lagarde, P., Chibon, F., Neuville, A., and Italiano, A. (2015). PRIMA-1(MET) induces death in soft-tissue sarcomas cell independent of p53. BMC cancer 15, 684.
Heredia-Moya, J., and Kirk, K. L. (2008). An improved synthesis of arsenic-biotin conjugates. Bioorganic & medicinal chemistry 16, 5743-5746.
Hu, J., Liu, Y. F., Wu, C. F., Xu, F., Shen, Z. X., Zhu, Y. M., Li, J. M., Tang, W., Zhao, W. L., Wu, W., et al. (2009). Long-term efficacy and safety of all-trans retinoic acid/arsenic trioxide-based therapy in newly diagnosed acute promyelocytic leukemia. Proceedings of the National Academy of Sciences of the United States of America 106, 3342-3347.
Joerger, A. C., and Fersht, A. R. (2007). Structure-function-rescue: the diverse nature of common p53 cancer mutants. Oncogene 26, 2226-2242.
Joerger, A. C., and Fersht, A. R. (2016). The p53 Pathway-Origins, Inactivation in Cancer, and Emerging Therapeutic Approaches. Annual review of biochemistry 85, 375-404.
Kandoth, C., McLellan, M. D., Vandin, F., Ye, K., Niu, B., Lu, C., Xie, M., Zhang, Q., McMichael, J. F., Wyczalkowski, M. A., et al. (2013). Mutational landscape and significance across 12 major cancer types. Nature 502, 333-339.
Khoo, K. H., Verma, C. S., and Lane, D. P. (2014). Drugging the p53 pathway: understanding the route to clinical efficacy. Nature reviews Drug discovery 13, 217-236.
Lambert, J. M., Gorzov, P., Veprintsev, D. B., Soderqvist, M., Segerback, D., Bergman, J., Fersht, A. R., Hainaut, P., Wiman, K. G., and Bykov, V. J. (2009). PRIMA-1 reactivates mutant p53 by covalent binding to the core domain. Cancer cell 15, 376-388.
Li, D., Marchenko, N. D., and Moll, U. M. (2011). SAHA shows preferential cytotoxicity in mutant p53 cancer cells by destabilizing mutant p53 through inhibition of the HDAC6-Hsp90 chaperone axis. Cell death and differentiation 18, 1904-1913.
Lindsley, R. C., Saber, W., Mar, B. G., Redd, R., Wang, T., Haagenson, M. D., Grauman, P. V., Hu, Z. H., Spellman, S. R., Lee, S. J., et al. (2017). Prognostic Mutations in Myelodysplastic Syndrome after Stem-Cell Transplantation. The New England journal of medicine 376, 536-547.
Liu, X., Wilcken, R., Joerger, A. C., Chuckowree, I. S., Amin, J., Spencer, J., and Fersht, A. R. (2013). Small molecule induced reactivation of mutant p53 in cancer cells. Nucleic acids research 41, 6034-6044.
Lo-Coco, F., Avvisati, G., Vignetti, M., Thiede, C., Orlando, S. M., lacobelli, S., Ferrara, F., Fazi, P., Cicconi, L., Di Bona, E., et al. (2013). Retinoic acid and arsenic trioxide for acute promyelocytic leukemia. The New England journal of medicine 369, 111-121.
Lu, M., Breyssens, H., Salter, V., Zhong, S., Hu, Y., Baer, C., Ratnayaka, I., Sullivan, A., Brown, N. R., Endicott, J., et al. (2013). Restoring p53 function in human melanoma cells by inhibiting MDM2 and cyclin B1/CDK1-phosphorylated nuclear iASPP. Cancer cell 23, 618-633.
Lu, M., Muers, M. R., and Lu, X. (2016a). Introducing STRaNDs: shuttling transcriptional regulators that are non-DNA binding. Nature reviews Molecular cell biology 17, 523-532.
Lu, M., Breyssens, H., Salter, V., Zhong, S., Hu, Y., Baer, C., Ratnayaka, I., Sullivan, A., Brown, N. R., Endicott, J., et al. (2013). Restoring p53 function in human melanoma cells by inhibiting MDM2 and cyclin B1/CDK1-phosphorylated nuclear iASPP. Cancer cell 23, 618-633.
Lu, M., Zak, J., Chen, S., Sanchez-Pulido, L., Severson, D. T., Endicott, J., Ponting, C. P., Schofield, C. J., and Lu, X. (2014). A code for RanGDP binding in ankyrin repeats defines a nuclear import pathway. Cell 157, 1130-1145.
Lu, T., Zou, Y., Xu, G., Potter, J. A., Taylor, G. L., Duan, Q., Yang, Q., Xiong, H., Qiu, H., Ye, D., et al. (2016b). PRIMA-1Met suppresses colorectal cancer independent of p53 by targeting MEK. Oncotarget.
Lukashchuk, N., and Vousden, K. H. (2007). Ubiquitination and degradation of mutant p53. Molecular and cellular biology 27, 8284-8295. Martins, C. P., Brown-Swigart, L., and Evan, G. I. (2006). Modeling the therapeutic efficacy of p53 restoration in tumors. Cell 127, 1323-1334.
Muller, P. A., Caswell, P. T., Doyle, B., Iwanicki, M. P., Tan, E. H., Karim, S., Lukashchuk, N., Gillespie, D. A., Ludwig, R. L., Gosselin, P., et al. (2009). Mutant p53 drives invasion by promoting integrin recycling. Cell 139, 1327-1341.
Muller, P. A., and Vousden, K. H. (2013). p53 mutations in cancer. Nature cell biology 15, 2-8.
Muller, P. A., and Vousden, K. H. (2014). Mutant p53 in cancer: new functions and therapeutic opportunities. Cancer cell 25, 304-317.
Parrales, A., Ranjan, A., lyer, S. V., Padhye, S., Weir, S. J., Roy, A., and Iwakuma, T. (2016). DNAJA1 controls the fate of misfolded mutant p53 through the mevalonate pathway. Nature cell biology 18, 1233-1243.
Patyka, M., Sharifi, Z., Petrecca, K., Mansure, J., Jean-Claude, B., and Sabri, S. (2016). Sensitivity to PRIMA-1MET is associated with decreased MGMT in human glioblastoma cells and glioblastoma stem cells irrespective of p53 status. Oncotarget.
Puca, R., Nardinocchi, L., Porru, M., Simon, A. J., Rechavi, G., Leonetti, C., Givol, D., and D'Orazi, G. (2011). Restoring p53 active conformation by zinc increases the response of mutant p53 tumor cells to anticancer drugs. Cell cycle 10, 1679-1689.
Riley, T., Sontag, E., Chen, P., and Levine, A. (2008). Transcriptional control of human p53-regulated genes. Nature reviews Molecular cell biology 9, 402-412.
Rippin, T. M., Bykov, V. J., Freund, S. M., Selivanova, G., Wiman, K. G., and Fersht, A. R. (2002). Characterization of the p53-rescue drug CP-31398 in vitro and in living cells. Oncogene 21, 2119-2129.
Shoemaker, R. H. (2006). The NCI60 human tumour cell line anticancer drug screen. Nature reviews Cancer 6, 813-823.
Soragni, A., Janzen, D. M., Johnson, L. M., Lindgren, A. G., Thai-Quynh Nguyen, A., Tiourin, E., Soriaga, A. B., Lu, J., Jiang, L., Faull, K. F., et al. (2016). A Designed Inhibitor of p53 Aggregation Rescues p53 Tumor Suppression in Ovarian Carcinomas. Cancer cell 29, 90-103.
Tessoulin, B., Descamps, G., Moreau, P., Maiga, S., Lode, L., Godon, C., Marionneau-Lambot, S., Oullier, T., Le Gouill, S., Amiot, M., et al. (2014). PRIMA-1Met induces myeloma cell death independent of p53 by impairing the GSH/ROS balance. Blood 124, 1626-1636.
Vassilev, L. T., Vu, B. T., Graves, B., Carvajal, D., Podlaski, F., Filipovic, Z., Kong, N., Kammlott, U., Lukacs, C., Klein, C., et al. (2004). In vivo activation of the p53 pathway by small-molecule antagonists of MDM2. Science 303, 844-848.
Ventura, A., Kirsch, D. G., McLaughlin, M. E., Tuveson, D. A., Grimm, J., Lintault, L., Newman, J., Reczek, E. E., Weissleder, R., and Jacks, T. (2007). Restoration of p53 function leads to tumour regression in vivo. Nature 445, 661-665.
Vogelstein, B., Lane, D., and Levine, A. J. (2000). Surfing the p53 network. Nature 408, 307-310.
Wang, Y., Suh, Y. A., Fuller, M. Y., Jackson, J. G., Xiong, S., Terzian, T., Quintas-Cardama, A., Bankson, J. A., El-Naggar, A. K., and Lozano, G. (2011). Restoring expression of wild-type p53 suppresses tumor growth but does not cause tumor regression in mice with a p53 missense mutation. The Journal of clinical investigation 121, 893-904.
Wassman, C. D., Baronio, R., Demir, O., Wallentine, B. D., Chen, C. K., Hall, L. V., Salehi, F., Lin, D. W., Chung, B. P., Hatfield, G. W., et al. (2013). Computational identification of a transiently open L1/S3 pocket for reactivation of mutant p53. Nature communications 4, 1407.
Weinmann, L., Wischhusen, J., Demma, M. J., Naumann, U., Roth, P., Dasmahapatra, B., and Weller, M. (2008). A novel p53 rescue compound induces p53-dependent growth arrest and sensitises glioma cells to Apo2L/TRAIL-induced apoptosis. Cell death and differentiation 15, 718-729.
Xue, W., Zender, L., Miething, C., Dickins, R. A., Hernando, E., Krizhanovsky, V., Cordon-Cardo, C., and Lowe, S. W. (2007). Senescence and tumour clearance is triggered by p53 restoration in murine liver carcinomas. Nature 445, 656-660.
Zhang, T. D., Chen, G. Q., Wang, Z. G., Wang, Z. Y., Chen, S. J., and Chen, Z. (2001). Arsenic trioxide, a therapeutic agent for APL. Oncogene 20, 7146-7153.
Zhang, X. W., Yan, X. J., Zhou, Z. R., Yang, F. F., Wu, Z. Y., Sun, H. B., Liang, W. X., Song, A. X., Lallemand-Breitenbach, V., Jeanne, M., et al. (2010). Arsenic trioxide controls the fate of the PML-RARalpha oncoprotein by directly binding PML. Science 328, 240-243.
Zhu, J., Chen, Z., Lallemand-Breitenbach, V., and de The, H. (2002). How acute promyelocytic leukaemia revived arsenic. Nature reviews Cancer 2, 705-713.
Chang CK, Zhao YS, Xu F, Guo J, Zhang Z, He Q, Wu D, Wu LY, Su JY, Song LX, Xiao C, Li X (2016). TP53 mutations predict decitabine-induced complete responses in patients with myelodysplastic syndromes. Br. J. Haematol. 176, 600-608.

1-67. (canceled)

68. A mp53 rescue compound, wherein the compound is a PANDA Agent.

69. The compound of claim 68, wherein the PANDA Agent is a compound selected from the group consisting of one or more three-valence arsenic compounds, five-valence arsenic compounds, three-valence bismuth compounds, five-valence bismuth compounds, three-valence antimony compounds, and five-valence antimony compounds.

70. The compound of claim 69, wherein the PANDA Agent excludes CP-31398; PRIMA-1; PRIMA-1-MET; SCH529074; Zinc; stictic acid, p53R3; methylene quinuclidinone; STIMA-1; 3-methylene-2-norbornanone; MIRA-1; MIRA-2; MIRA-3; NSC319725; NSC319726; SCH529074; PARP-PI3K; 5, 50-(2, 5-furandiyl) bis-2-thiophenemethanol; MPK-09; Zn-curc or curcumin-based Zn (II)-complex; P53R3; a (2-benzofuranyl)-quinazoline compound; a nucleolipid compound of 5-fluorouridine; a compound of 2-aminoacetophenone hydrochloride; PK083; PK5174; and PK7088.

71. A mp53 rescue compound comprising M, wherein M is selected from the group consisting of one or more three-valence arsenic, five-valence arsenic, three-valence bismuth, five-valence bismuth, three-valence antimony, and five-valence antimony.

72. The compound of claim 71, wherein the group M is capable of forming one or more tight associations with one or more PANDA Cysteines.

73. The compound of claim 71, having one or more of the following formula:
M (Formula I), M-Z (Formula II), wherein:
M is an atom selected from a group consisting of As, Sb, and Bi;
Z is a functional group comprising a non-Carbon atom that forms a bond with M,
wherein the non-Carbon atom is selected from the group consisting of H, D, F, Cl, Br, I, O, S, Se, Te, Li, Na, K, Cs, Mg, Cu, Zn, Ba, Ta, W, Ag, Cd, Sn, X, B, N, P, Al, Ga, In, Tl, Ni, Si, Ge, Cr, Mn, Fe, Co, Pb, Y, La, Zr, Nb, Pr, Nd, Sm, Eu, Gd, Dy, Tb, Ho, Er, Tm, Yb, and Lu;
wherein:
R1 is selected from 1 to 9 X groups;
R2 is selected from 1 to 7 X groups;
R3 is selected from 1 to 8 X groups; and wherein each X group comprises an atom that forms a bond with M; and
wherein:
each of M, the non-Carbon atom, and the atom has the appropriate charge, including no charge, in the compound;
each of Z and X is independently selected and can be the same or different from the other Z or X in the compound, respectively; and
each of the M, non-Carbon atom and the atom can be a part of a ring member.

74. The compound of claim 73, wherein the non-Carbon atom is selected from the group consisting of O, S, N, X, F, Cl, Br, I, and H.

75. A mp53 rescue compound, wherein the compound is selected from Table 1 to Table 7.

76. The compound of claim 75, wherein the compound is selected from the group consisting of As2O3, As2O5, KAsO2, NaAsO2, HAsNa2O4, HAsK2O4, AsF3, AsCl3, AsBr3, AsI3, AsAc3, As (OC2H5)3, As (OCH3)3, As2 (SO4)3, (CH3CO2)3As, C8H4K2O12As2.xH2O, HOC6H4COOAsO, [O2CCH2C (OH) (CO2) CH2CO2] As, Sb2O3, Sb2O5, KSbO2, NaSbO2, HSbNa2O4, HSbK2O4, SbF3, SbCl3, SbBr3, SbI3, SbAc3, Sb (OC2H5)3, Sb (OCH3)3, Sb2(SO4)3, (CH3CO2)3Sb, C8H4K2O12Sb2.xH2O, HOC6H4COOSbO, [O2CCH2C(OH)(CO2)CH2CO2]Sb, Bi2O3, Bi2O5, KBiO2, NaBiO2, HBiNa2O4, HBiK2O4, BiF3, BiCl3, BiBr3, BiI3, BiAc3, Bi(OC2H5)3, Bi(OCH3)3, Bi2(SO4)3, (CH3CO2)3Bi, C8H4K2O12Bi2.xH2O, HOC6H4COOBiO, C16H18As2N4O2(NSC92909), C13H14As2O6 (NSC48300), C10H13NO8Sb (NSC31660), C6H12NaO8Sb+(NSC15609), C13H21NaO9Sb+(NSC15623), and a combination thereof.

77. The compound of claim 75, wherein the compound is selected from the group consisting of As2O3, KAsO2, HOC6H4COOBiO, BiI3, SbI3, C8H4K2O12Sb2.H2O, As2S2, As4S4, As2S3, and As2S5.

78. A pharmaceutical composition for a p53 disorder comprising the compound of claim 73 and a non-toxic, pharmaceutically acceptable carrier or excipient therefor.

79. The pharmaceutical composition of claim 78, wherein the pharmaceutical composition is formulated for orally administering.

80. The pharmaceutical composition of claim 79, wherein the compound is selected from the group consisting of As2S3, As2S2, and As2S5.

81. The pharmaceutical composition of claim 78 further comprising at least one compatible therapeutic agent for p53 disorder, wherein the therapeutic is effective in treating the p53 disorder.

82. The pharmaceutical composition of claim 81, wherein the compatible therapeutic agent for p53 disorder is selected from the group consisting of decitabine (“DAC”), cisplatin (“CIS”), etoposide (“ETO”), adriamycin (ADM”), 5-fluorouracil (“5-FU”), cytarabine (“ARA/araC”) and azacitidine (“AZA”).

83. A method of treating a p53 disorder in a subject in need thereof, the method comprising the steps:
(a) obtaining a sample from the subject; and
(b) administering a pharmaceutical composition of claim 78 to the subject if the sample has a p53 mutation.

84. A method of detecting a rescuable mp53 in a subject comprising the steps:
(a) obtaining a sample from the subject;
(b) adding a PANDA agent of claim 70 to the sample; and
(b) identifying the presence of the rescuable mp53 in the subject if, in the presence of the PANDA Agent (i) the PAb1620 immunoprecipitation signal increase to 1.5 times or more and/or (ii) the luciferase assay of a signal increase to 1.5 times or more.

85. A method of identifying the presence of a rescuable mp53 in a subject comprising the steps:
(a) obtaining a sample from the subject;
(b) adding a PANDA Agent of claim 70 to a first portion of the sample; and
(c) identifying the presence of a rescuable mp53 in the subject if immunoprecipitation signal of the first portion by PAb1620 at greater than 4° C. is 1.5 times or more than the immunoprecipitation signal of the second portion of the sample without the PANDA Agent.

86. A method of diagnosing and treating a subject with a p53 disorder comprising the steps:
(a) obtaining a sample from the subject;
(b) diagnosing the subject is suitable for the pharmaceutical composition of claim 78 if the sample has a rescuable p53 mutation; and
(c) treating the subject by administering a pharmaceutical composition of claim 78 to the subject if the sample has a rescuable mp53.

87. A method of personalized treatment for a p53 related disorder in a subject in need thereof with increased efficacy, the method comprising the steps of:
(a) obtaining a p53 DNA sample from the subject;
(b) sequencing the p53 DNA sample;
(c) determining whether the p53 of the subject is rescuable and identifying one or more PANDA Agent and/or a combination of PANDA Agent of claim 70 that is most appropriate to rescue the p53 in the subject; and
(d) administering an effective amount of the PANDA Agent and/or the combination of PANDA Agent to the subject;
wherein step (c) includes the step (s) (i) determining in silico whether the sequence of the p53 DNA sample is comparable to a to a database of rescuable p53s and identifying the corresponding PANDA Agent (s) and/or combination of PANDA Agents most appropriate to rescue the p53 using the database; and/or (ii) determining in vitro and/or in vivo whether the p53 of the subject can be rescued by screening it against a panel of PANDA Agents.
